(19) United States
(12) Patent Application Publication
(10) Pub. No.: US 2025/0367649 A1
(43) Pub. Date: Dec. 4, 2025

(54) PHOTOCATALYSTS, PREPARATION AND USE THEREOF

(71) Applicant: THE ROYAL INSTITUTION FOR THE ADVANCEMENT OF LEARNING/MCGILL UNIVERSITY, Montréal (CA)

(72) Inventors: Chao-Jun LI, Brossard (CA); Jianbin LI, Jiangmen (CN); Chia-Yu HUANG, Taipei City (TW)

(21) Appl. No.: 18/707,916

(22) PCT Filed: Nov. 1, 2022
(86) PCT No.: PCT/CA2022/051611

§ 371 Date: May 7, 2024

Related U.S. Application Data
(60) Provisional application No. 63/276,848, filed on Nov. 8, 2021.

(51) Int. Cl. B01J 31/18; B01J 35/39; C07C 2/58

(52) U.S. Cl. CPC B01J 31/1815; B01J 35/39; C07C 2/58; B01J 2231/44; B01J 2531/845; C07C 2531/12; C07C 2531/18; C07C 2531/28

(57) ABSTRACT

There is provided a process for alkylating a substrate with a photocatalytic system. The process comprises providing a mixture containing an acid, and a substrate (an organic compound). Then, an organophotoredox catalyst of formula Ia is contact with the mixture. Finally, the organophotoredox catalyst is activated with a light irradiation to alkylate the substrate and form a carbon covalent bond.

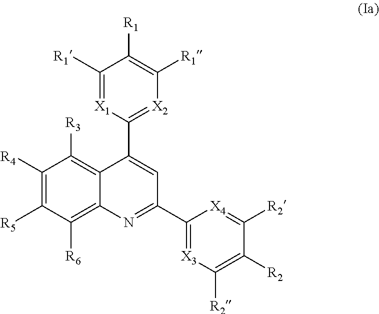

### TECHNICAL FIELD

This disclosure relates to the field of organic photocatalysts, their preparation and usages.

### BACKGROUND OF THE ART

The catalytic proficiency of photocatalysts to effect carbon radical generation has revolutionized the manner in which chemists conceive and elicit novel reactions. In this context, the advent of polypyridyl metallocomplexes of iridium and ruthenium enlightened a wide range of photochemical approaches to forge C—C and C—X bonds among many other chemical transformations. Concerning their high costs and potential toxicity, more sustainable organic dyes and some well-tailored organic-based photocatalysts were introduced. Unfortunately, organic dyes often suffer narrow redox windows and poor solubility. Many commercialized organophotocatalysts are structurally sophisticated, therefore, necessitating prolonged and inconvenient synthesis.

Under photocatalyzed conditions, redox-neutral C—C cross-couplings exemplify one of the most common transformations, which formally pair a carbon nucleophile and an electrophile. To move beyond this paradigm and realize cross-nucleophile couplings, a stoichiometric amount of oxidants are often mandated, which inevitably requires some extent of screening to maximize productivity.

Taking Minisci alkylation as an example, since the milestone discovery by Minisci's group, it has become one of the most privileged C—H functionalization protocols for heteroaromatic scaffolds via carbon radical intermediates. Given the competence of photocatalysts in mediating redox steps, marrying photoredox catalysis with Minisci reactions represents a fundamental advancement in various settings. However, their conditions often consist of costly photocatalysts and stoichiometric chemical oxidants that were either situated as exogenous additives or embedded in the reactants. In contrast, net-oxidation Minisci-type transformations that bypass these oxidizing components with their chemical equivalents, preferably in catalytic quantity, remain underexplored. Along this line, hydrogen evolution provides a paradigm-shifting alternative that could not only realize the redox adjustment but also drive the overall reaction progress. In this context, electrochemistry and semiconductors have been shown as enabling tools for releasing hydrogen. However, further improvements to these costly and complicated methods are desired. Indeed, it would be advantageous to use a homogenous catalyst to improve the efficiency and cost of the synthesis, by for example eliminating the need for electrochemistry or semiconductors.

### SUMMARY

In one aspect, there is provided a photocatalyst that catalyzes the formation of covalent bonds. The photocatalyst is activated by protonation of its quinoline nitrogen and light irradiation. The photocatalyst of the present disclosure can be grafted on a larger molecule, a polymer or a solid support with a chemical linker. The photocatalyst can be an organophotoredox catalyst as described further herein below.

In one aspect, there is provided a method for alkylating a substrate with a photocatalytic system, the process comprising: providing a mixture comprising an acid, and the substrate being an organic compound; contacting an organophotoredox catalyst according to the present disclosure with the mixture; and activating the organophotoredox catalyst with a light irradiation to alkylate the substrate and form a carbon covalent bond. In some embodiments, the organophotoredox catalyst has a quinoline core substituted at positions C2 and/or C4 by aryl or heteroaryl groups, and at least one of the aryl or heteroaryl groups is substituted. In some embodiments, at least one of the aryl or heteroaryl group is substituted with an electron donating group such as an alkyl group (weak electron donating group) or a group containing O, N or S. In some embodiments, the aryl is a C6-C10 aryl group. In some embodiments, the heteroaryl group is a C5-C10 heteroaryl group. In still further embodiments, the aryl group is a phenyl and the heteroaryl group is a C5 heteroaryl. In additional embodiments, the heteroatom of the heteroaryl is nitrogen.

In one aspect, there is provided a process for alkylating a substrate with a photocatalytic system, the process comprising:
providing mixture comprising an acid, and the substrate;
contacting an organophotoredox catalyst of formula Ia with the mixture

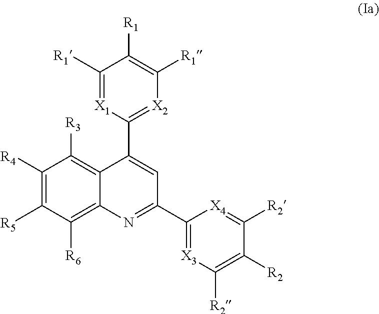

where R₁, R₁′, R₁″, R₂, R₂′, R₂″, R₃, R₄, R₅, R₆, X₁, X₂, X₃, and X₄, are as defined herein and
activating the organophotoredox catalyst with a light irradiation to alkylate the substrate and form a carbon covalent bond.

Many further features and combinations thereof concerning the present improvements will appear to those skilled in the art following a reading of the instant disclosure.

### DESCRIPTION OF THE DRAWINGS

FIG. 1 is a chemical structure of 2,4-di-(4-methoxyphenyl)quinoline (DPQN^{2,4-di-OMe}) generated by X ray analysis.

FIG. 2A is a spectroscopic characterization of 2,4-di-(4-methoxyphenyl)quinoline (DPQN^{2,4-di-OMe}) by UV-vis and fluorescence.

FIG. 2B is a cyclic voltammogram of DPQN^{2,4-di-OMe}, and DPQN^{2,4-di-OMe} with an equimolar amount of trifluoroacetic acid (TFA).

FIG. 2C is a graph showing the quenching of DPQN^{2,4-di-OMe} (intensity in function of wavelength of light irradiation) with 0.5 mM DPQN^{2,4-di-OMe}, 0.5 mM TFA, and (i) 0.025 μM cyclohexyl trifluoroborate potassium (Cy-BF₃K), (ii) 0.050 μM Cy-BF₃K, (iii) 0.075 μM Cy-BF₃K, or (iv) 0.100 μM Cy-BF₃K.

FIG. 2D is a graph showing the absorption decay for an equimolar amount of DPQN^{2,4-di-OMe} and TFA.

FIG. 3A is a photograph comparing photophysical properties of a 10 mM solution of: a: DPQN^{2,4-di-OMe}; b: DPQN^{2,4-di-OMe}+TFA (1:1 molar); c: diphenylquinoline (DPQN)+TFA (1:1 molar); d: 2-(4-trifluoromethylphenyl)-4-phenylquinoline (DPQN^{2-CF3})+TFA (1:1 molar), under ambient light and under Kessil light (390 nm light irradiation).

FIG. 3B is a graph of the absorbance in function of the concentration for DPQN^{2,4-di-OMe} (+), DPQN^{2,4-di-OMe} & TFA (1:1 molar) (ϕ), DPQN^{2-CF3} & TFA (1:1 molar) (x), and DPQN & TFA (1:1 molar) (−).

FIG. 3C is a fluorescence spectra (intensity as a function of wavelength) for DPQN^{2,4-di-OMe} DPQN^{2,4-di-OMe} & TFA (1:1 molar), DPQN^{2-CF3} & TFA (1:1 molar), and DPQN & TFA (1:1 molar).

FIG. 3D is a Stern-Volmer plot of DPQN^{2,4-di-OMe} (●), DPQN^{2,4-di-OMe} & TFA (1:1 molar) (▴), DPQN^{2-CF3} & TFA (1:1 molar) (▪), and DPQN & TFA (1:1 molar) (x).

FIG. 4 shows a graph of the light on/off experiment showing the conversion percentage in function of time.

FIG. 5 shows an electron paramagnetic resonance (EPR) spectra for DPQN^{2,4-di-OMe} in the dark, with light, and a simulation.

FIG. 6 is a schematic representation of the structure of PPQN^{2,4-di-OMe}.

FIG. 7 is a schematic representation of the structure of Ni²⁺/PPQN^{2,4-di-OMe}.

FIG. 8A is an ultraviolet-visible (UV-vis) spectrum showing the intensity in function of the wavelength for nickel species.

FIG. 8B is a UV-vis spectrum showing the intensity in function of the wavelength for copper species.

FIG. 8C is a UV-vis spectrum showing the intensity in function of the wavelength for cobalt species.

FIG. 8D is a UV-vis spectrum showing the intensity in function of the wavelength for iron species.

FIG. 9A is a cyclic voltammogram showing the current in function of potential for nickel species.

FIG. 9B is a cyclic voltammogram showing the current in function of potential for copper species.

FIG. 9C is a cyclic voltammogram showing the current in function of potential for cobalt species.

FIG. 9D is a cyclic voltammogram showing the current in function of potential for iron species.

FIG. 10A shows a representation of the solid-state structure of Ni²⁺/PPQN^{2,4-di-OMe}. The ellipsoids were drawn at 50% probability. The H₂O molecule and all the hydrogens in the X-ray structures were omitted for clarity.

FIG. 10B shows the results of density functional theory (DFT) calculations on the structure of Ni²⁺/(PPQN^{2,4-di-OMe})Cl₂ with highest occupied molecular orbital (HOMO).

FIG. 10C shows the results of DFT calculations on the structure of Ni²⁺/(PPQN^{2,4-di-OMe})Cl₂ with lowest occupied molecular orbital (LOMO).

FIG. 10D is a schematic top view of the structure Ni(PPQN^{2,4-di-OMe})Cl₂.

FIG. 10E is a schematic front view of the structure Ni(PPQN^{2,4-di-OMe})Cl₂.

### DETAILED DESCRIPTION

There is provided a cost-effective organophotoredox catalyst that is an efficient, low-cost, homogeneous co-catalyst to perform chemical reactions such as an alkylation, for example a Minisci alkylation. The organophotoredox catalyst of the present disclosure has a simple photoactivation mechanism, and has reduced sensitive functionalities and byproduct formation. The organophotoredox catalyst of the present disclosure does not require laborious and expensive electrochemical systems or semiconductors to perform an alkylation such as a Minisci alkylation.

The terms “alkylating”, “alkylation” and the like, as used herein refer to a chemical reaction that forms a covalent carbon bond or that grafts a chemical structure to a substrate using a carbon covalent bond. The carbon covalent bond may be a C—C bond, a C—O bond, a C—N bond or a C—S bond. In some embodiments, the carbon covalent bond is a single bond. The alkylation can also occur within a compound, for example a cyclisation of a compound that would result in the formation of a carbon covalent bond within the same molecule, such as a C—C bond. Many different types of alkylations are contemplated by the present disclosure including but not limited to alkyne additions, group transfers, alkyl addition (e.g. to a nitrogen or sulfur of a substrate) and Minisci alkylations. A Minisci alkylation is type of alkylation in which a radical reaction that introduces an alkyl group to an electron deficient aromatic heterocycle occurs. In some embodiments, the heterocycle is a heterocycle containing a nitrogen. In further embodiments, the heterocycle is a quinoline group, a pyridine group, an indole group or an acridine group.

Unlike the prior art photocatalysts, which impart their photoreactivities as covalently linked entities, the present organophotoredox catalyst has a distinct activation that is a proton activation mode or a Lewis acid coordination activation mode. Simply upon protonation, the organophotoredox catalyst reaches an oxidizing excited state. The protonation may be activated by a suitable acid and following protonation light irradiation, for example a visible light irradiation catalyzes the alkylation. In some embodiments, the light irradiation has a wave length of from 380 to 780 nm, of from 380 to 680 nm, or of from 380 to 580 nm. The organophotoredox catalyst can be employed alone or in combination with one or more co-catalysts such as metal organocatalysts. In some embodiments, the alkylation is a Minisci alkylation and the organophotoredox catalyst is combined with a cobalt organocatalyst such as a cobaloxime (e.g. chloro(pyridine)cobaloxime) to formulate an oxidative cross-coupling platform, enabling alkylation reactions such as Minisci alkylations and various C—C bond-forming reactions. In some embodiments, the present disclosure does not contemplate the addition of any other chemical oxidants.

The organophotoredox catalyst of the present disclosure has a chemical structure according to formula Ia.

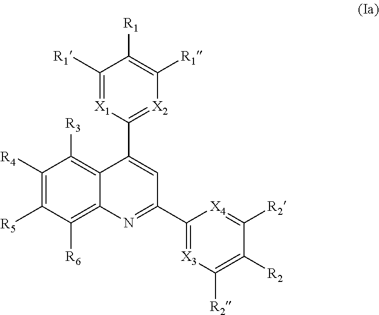

R₁, R₁′, R₁″ are independently selected from hydrogen, substituted or unsubstituted alkyl, substituted or unsubstituted X-alkyl, chemical linker, or X-chemical linker with X being one of an oxygen, an amine or a sulfur. X₁, and X₂ are independently selected from CH or N. When X₁ is N, X₂ is CH, R₁ and R₁′ are hydrogen. When X₂ is N, X, is CH, R₁ and R₁″ are hydrogen. When X₁, and X₂ are both CH, R₁′ and R₁″ are hydrogen.

R₂, R₂′, R²″ are independently selected from hydrogen, substituted or unsubstituted alkyl, substituted or unsubstituted X-alkyl, chemical linker, or X-chemical linker with X being one of an oxygen, an amine or a sulfur. X₃, and X₄ are independently selected from CH or N. When X₃ is N, X₄ is CH, R₂ and R²″ are hydrogen. When X₄ is N, X₃ is CH, R₂ and R₂′ are hydrogen. When X₃, and X₄ are both CH, R₂′ and R²″ are hydrogen.

In some embodiments, R¹, R₁′, R₁″, R₂, R₂′, R²″ are not all hydrogen unless X₃ is N. In some embodiments, R₁, R₁′, R₁″, R₂, R₂′, R²″ are not all hydrogen. In some embodiments, at least one of R₁, R₁′, R₁″, R₂, R₂′, R²″ has or is an electron donating group to promote and facilitate the protonation of the nitrogen of the quinoline ring. In some embodiments, an alkyl group is a weak electron donating group that is sufficient to promote the protonation of the nitrogen of the quinoline ring. In some embodiments, at least one of X₁, X₂ X₃, and X₄ is N.

R₃, R₄, R₅, and R₆ are independently selected from hydrogen, substituted or unsubstituted alkyl, substituted or unsubstituted alkenyl, substituted or unsubstituted cycloalkyl, substituted or unsubstituted cycloalkenyl, substituted or unsubstituted aryl, substituted or unsubstituted heteroaryl, substituted or unsubstituted heterocyclyl.

The term “alkyl”, as used herein, is understood as referring to a saturated, monovalent unbranched or branched hydrocarbon chain. In some embodiments, the alkyl can be the backbone of a polymer such as polystyrene. In other embodiments, the alkyl group can comprise up to 20 carbon atoms. Examples of alkyl groups include, but are not limited to, C₁-C₁₀ alkyl groups, provided that branched alkyls comprise at least 3 carbon atoms, such as C₃-C₁₀. Lower straight alkyl may have 1 to 6 or 1 to 3 carbon atoms; whereas branched lower alkyl comprise C₃-C₆. Examples of alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, tert-butyl, pentyl, isopentyl, neopentyl, hexyl, heptyl, octyl, nonyl and decyl. In some embodiments, the term “alkyl” in the context of the present disclosure and particularly for groups R₁ and R₂ is further defined to exclude alkyl groups with one or more hydrogen atom being replaced by a halogen, ie. a haloalkyl.

The term “alkylenyl”, as used herein, is understood as referring to bivalent alkyl residue. Examples of alkylenyl groups include, but are not limited to, ethenyl, propenyl, 2-methyl-1-propenyl, 2-methyl-2-propenyl, 2-methyl-1-butenyl, 3-methyl-1-butenyl, 2-methyl-3-butenyl, 2-methyl-1-pentenyl, 3-methyl-1-pentenyl, 4-methyl-1-pentenyl, 2-methyl-2-pentenyl, 3-methyl-2-pentyl, 4-methyl-2-pentyl, 2-ethyl-1-butenyl, butenyl, pentenyl, hexenyl, heptenyl, octenyl, nonenyl and decenyl.

The term “cycloalkyl” represents a cyclic hydrocarbon moiety having 3 to 10 carbon atoms. Cycloalkyl may be a monocyclic hydrocarbon moiety having 3 to 8 carbon atoms. Examples of “cycloalkyl” groups include but are not limited to cyclopropyl, cyclobutyl, cyclopentyl, cyclohexyl, cyclohexenyl and cyclooctyl. The cycloalkyl group can be a polycyclic group for example a polycyclic group having 7 to 10 carbons. For example, the cycloalkyl can be a bicycloalkyl such as bicycloheptane. In a further example, the cycloalkyl can be a tricycloalkyl such as adamantanyl. In an additional example, the cycloalkyl can be a multicyclic alkyl such as cubanyl.

The term “cycloalkenyl” is a cycloalkyl group which has one or more double bonds, preferably one double bond. Examples of cycloalkenyl include but are not limited to cyclopentenyl, cyclohexenyl, and cycloheptenyl.

The term “aryl” represents a carbocyclic moiety containing at least one benzenoid-type ring (i.e., may be monocyclic or polycyclic). Preferably, the aryl comprises 6 to 10 or more preferably 6 carbon atoms. Examples of aryl include but are not limited to phenyl and naphthyl.

The term “heteroaryl” represents an aryl having one or more carbon in the aromatic ring(s) replaced by nitrogen. The heteroaryl can have 3 to 9 carbon atoms (C₃-C₉) with the remainder atoms of the aromatic ring(s) being nitrogen. Examples of heteroaryl include but are not limited to pyridinyl, pyrazinyl, pyrimidinyl, pyridazinyl, triazinyl, quinolinyl, quinoxalinyl, quinazonyl, cinnolinyl, triazolopyridinyl, trioazolopyrimidinyl, diaazolopyrimidinyl, diazolopyridinyl, and triazynyl.

The term “heterocyclyl” represents a 3 to 10 membered saturated (heterocycloalkyl), partially saturated (heterocycloalkylene), and any other heterocyclic ring that can be aromatic or non-aromatic. The heterocyclyl comprises at least one heteroatom selected from oxygen (O), sulfur (S), silicon (Si) or nitrogen (N) replacing a carbon atom in at least one cyclic ring. Heterocyclyl may be monocyclic or polycyclic rings. Heterocyclyl may be 3 to 8 membered monocyclic ring. The heterocyclyl ring, in some examples, can contain only 1 carbon atom (for example tetrazolyl). Therefore the heterocyclyl can be a C₁-C₇ heterocyclyl. When heterocyclyl is a polycyclic ring, the rings comprise at least one heterocyclyl monocyclic ring and the other rings may be fused cycloalkyl, aryl, heteroaryl or heterocyclyl and the point of attachment may be on any available atom or pair of atoms. Examples of heterocycloalkyl include but are not limited to piperidinyl, oxetanyl, morpholino, azepanyl, pyrrolidinyl, azetidinyl, azocanyl, and azasilinanyl. Examples of heterocycloalkylene include but are not limited to dihydropyranyl, dihydrothiopyranyl, and tetrahydropiperidine. Examples of further monocyclic heterocyclyl include but are not limited to azolyl, diazolyl, triazolyl, tetrazolyl, oxazolyl, isoxazolyl, thiophenyl, furanyl, thiazolyl, and isothiazolyl. Examples of polycyclic heterocyclyl include but are not limited to oxa-azabicyclo-heptanyl, oxa-azaspiro-heptanyl, azabicyclo-hexanyl, azaspiro-heptanyl, dihydroquinolinyl, and azaspiro-octanyl.

The term “substituted” or “substituent” represents at each occurrence and independently, one or more oxide, amino, amidino, amido, azido, cyano, guanido, hydroxyl, nitro, nitroso, carbonitrile, urea, alkyl, alkoxy, carboxy (i.e. —COOH), alkyl-carboxy (i.e. alkyl substituted with COOH), ester, alkyl as defined herein, alkenyl as defined herein, cycloalkyl as defined herein, aryl as defined herein, heteroaryl as defined herein, or heterocyclyl as defined herein. The substituents of the present disclosure may replace a hydrogen of a carbon of the carbon backbone of a substituted chemical species and/or can interrupt the carbon backbone of the substituted species. For example, a nitrogen may replace a hydrogen resulting in a —CH₂—CH(NH₂)—CH₂— or can interrupt the chain to result in —CH₂—NH₂—CH₂—.

The term “chemical linker” as used herein refers to a covalent chemical linker that binds to the organophotoredox through R₁ or R₂. The chemical linker can for example be a linker that immobilizes the organophotoredox of the present disclosure to a surface, such as the surface of a bead. The chemical linker may be linked to any suitable functional group. In one example, the functional group can be part of a polymer. The chemical linker of the present disclosure can contain maleimide, sulfhydryl reactive groups, or succinimidyl esters which react with amines. Other suitable chemical linkers are contemplated by the present disclosure as long as the chemical linkers do not interfere with the alkylation reaction.

In some embodiments, the organophotoredox catalyst of the present disclosure is of formula Ib with R₁, R₂, R₃, R₄, R₅, and R₆ as previously defined herein and X₃ being N or CH. R₁ and R₂ are not both H when X₃ is CH.

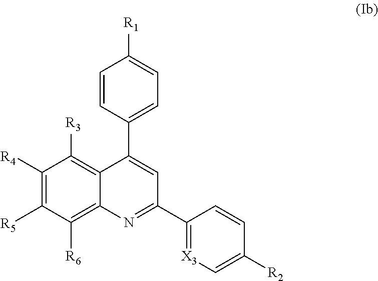

In still further embodiments, the organophotoredox catalyst of the present disclosure is of formula Ic with R₁, and R₂ as previously defined herein and X₃ being N or CH. R₁ and R₂ are not both H when X₃ is CH.

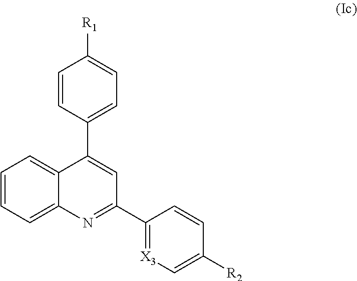

In yet further embodiments, the organophotoredox catalyst has a chemical structure according to formula Id with R₁ and R₂ being as previously defined herein. In one example, R₁ and R₂ are each independently selected from —H, -Me, —OMe, -(chemical linker) and —O— (chemical linker), and R₁ and R₂ are not both —H.

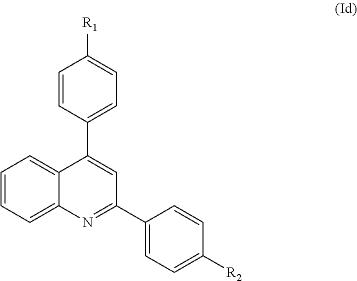

In some embodiments, the organophotoredox catalyst is selected from the group consisting of

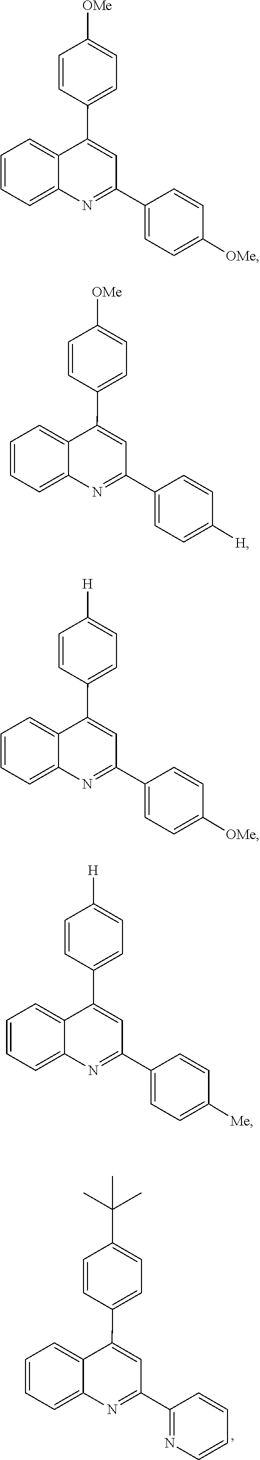

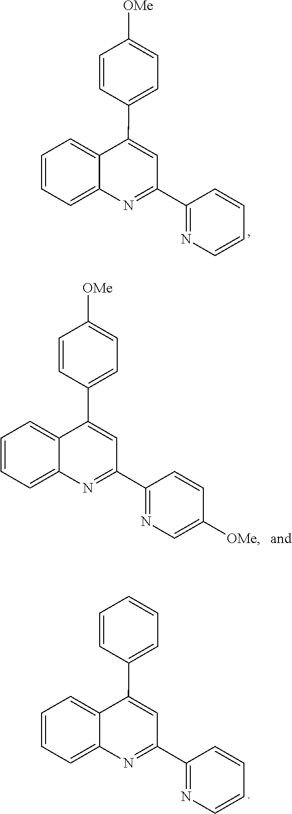 [SMILES: COc1ccc(-c2cc(-c3ccccn3)nc3ccccc23)cc1 ; COc1ccc(-c2cc(-c3ccc(OC)cn3)nc3ccccc23)cc1 ; "and" ; c1ccc(-c2cc(-c3ccccn3)nc3ccccc23)cc1]

The organophotoredox catalyst of formulas Ia, Ib, Ic, and Id is activated by protonation of the nitrogen of the quinolone group. Accordingly, once protonated, the activated organophotoredox catalyst of formula Ia becomes formula IIa, formula Ib becomes formula IIb, formula Ic becomes formula IIc and formula IId becomes formula IId. The definitions of the substituent groups of formulas Ia, Ib, Ic, and Id respectively apply to formulas IIa, IIb, IIc, and IId.

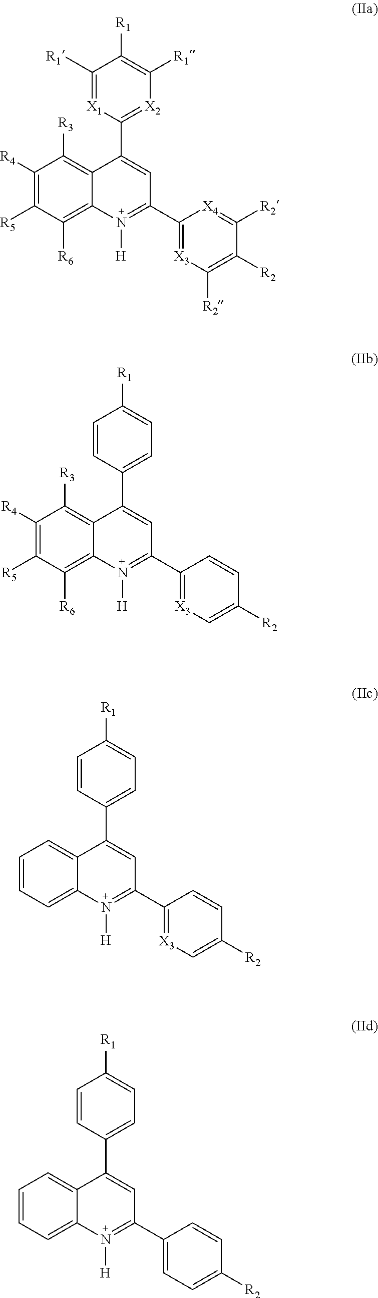

The organophotoredox catalyst furnishes carbon radicals from an array of attractive precursors and can for example complete the Minisci alkylation when partnered with a cobaloxime chaperone. Moreover, the pronounced photosynthetic capacity of the present catalytic system can be used in other oxidative cross-coupling reactions for carbon bond formations, such as oxidative arene fluoroalkylation and alkene/alkyne dicarbofunctionalization.

There is provided a process of alkylating a substrate, the process comprises providing a mixture that includes an acid, the substrate and optionally a cobalt, nickel, copper or iron co-catalyst. The metal containing co-catalyst can be elemental or ionic cobalt, nickel, copper or iron, or a molecule containing cobalt, nickel, copper or iron. For example, the co-catalyst can be an organic metallocatalyst such as chloro(pyridine)cobaloxime. The process comprises contacting the organophotoredox catalyst as described herein with the mixture. The co-catalyst, such as a cobalt organophotoredox catalyst, can be included in the mixture or can be linked on a surface or solid substrate through a chemical linker group at R₁ and/or R₂ and brought into contact with the reaction. For example, the organophotoredox catalyst can be linked to a polystyrene (PS) bead or any other suitable catalytic surface with the chemical linker at R₁ and/or R₂. The process further comprises activating the organophotoredox catalyst with a light irradiation to alkylate the substrate and form a C—C covalent bond. The substrate is an organic compound preferably containing multiple C—H bonds (for example at least 3, preferably at least 5 and more preferably at least 10). In some embodiments, the substrate is an organic compound having a molecular weight of from 50 to 1000 g/mol. In further embodiments, the substrate is an organic compound comprising at least one cyclic group, for example an aromatic cyclic group. In some embodiments the substrate is a compound containing at least 1, at least 2, at least 3, at least 4 or at least 5 carbon atoms each having at least one C—H bond. In some embodiments, the substrate is solid or liquid at room temperature. The substrate is a compound capable of performing an alkylation reaction with another compound or with itself (e.g. cyclization reaction).

The organophotoredox catalyst is also provided as a metallophotoredox catalyst. The organophotoredox catalyst can form a metal containing compound with the co-catalyst (i.e. metallophotoredox catalyst). In such embodiments, the organophotoredox catalyst is of formula Ia, Ib, or Ic with X₃ being N and the metal is a redox active metal. Preferably, the redox active metal is a Lewis acidic transition metal. More preferably, the redox active metal is selected from Ni, Co, Cu or Fe. The metallophotoredox catalyst formed is shown in formulas Ie, If, and Ig with M representing the redox active metal which is preferably Ni, Co, Cu or Fe. The redox active metal M forms donor-acceptor coordination bonds with the nitrogen atoms. In formula Ie, R₁, R₁′, R₁″, R₂, R₂′, R²″, R₃, R₄, R₅, R₆, X₁, and X₂ are as previously defined for formula Ia. In formula If, R₁, R₂, R₃, R₄, R₅, R₆ are as previously defined for formula Ib. In formula Ig, R₁, R₂ are as previously defined for formula Ic. The metallophotoredox is formed by stirring a compound containing the redox active metal with the organophotoredox catalyst of formula Ia, Ib, or Ic with X₃ being N, preferably in a molar ratio of 1:2 to 2:1, and more preferably in equimolar amounts.

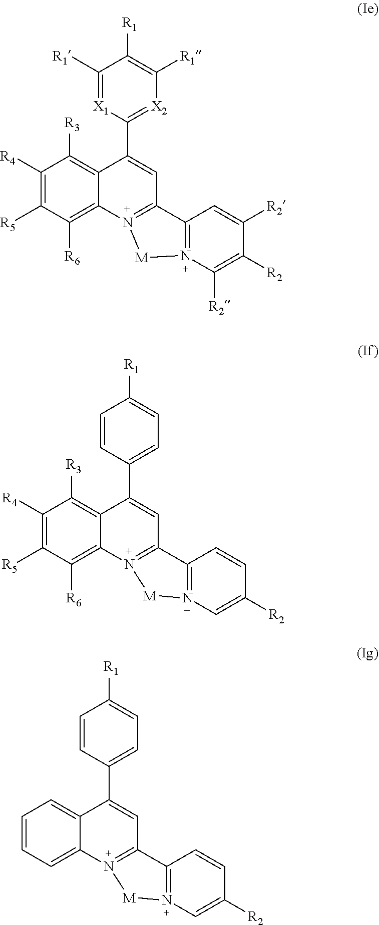

In some embodiments, the process of the present disclosure is performed under inert atmosphere. An inert atmosphere is an atmosphere that will not significantly interfere with the alkylation reaction or the protonation of the organophotoredox. In some embodiments, the inert atmosphere is a gas atmosphere such as N₂, Ar, He, Ne, Kr, orXe. In some embodiments, a co-catalyst is selected from a cobalt catalyst (such as cobalt organocatalyst), a copper catalyst, an iron catalyst or a nickel catalyst. The cobalt organocatalyst may be a cobaloxime such as chloro(pyridine)cobaloxime. In one example, the cobalt organocatalyst is chloro(pyridine)bis(dimethylglyoximato)cobalt (III).

In some embodiments, the acid is trifluoroacetic acid (TFA) or HCl. However the choice of acid will depend on the type of alkylation and co-catalyst when used. In some embodiments, the role of the acid is to promote the protonation of the nitrogen of the quinoline group of the organophotoredox catalyst.

An alkylation precursor may be provided in the mixture in order to link an alkylation group of the precursor to the substrate. Examples of alkylation precursors include but are not

limited to trifluoroborate salts such as the potassium salt,

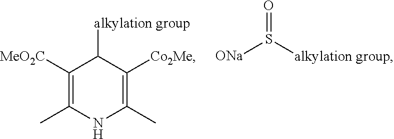

Boc₂(NH)—SO₂-(alkylation group) (Boc=tert-butyloxycarbonyl), NH₃COO-(alkylation group),

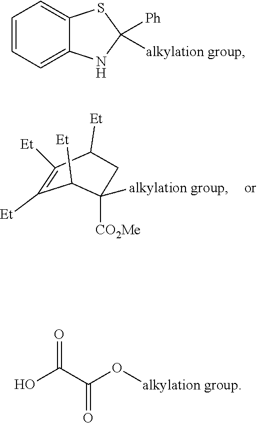

### Example 1

Conjugated heteroaromatic motifs, especially N-heterocycles, are frequently seen in photocatalytic chromophores (formulas III, IV, V). Indeed, isolated heteroarenes, for instance, quinolines, have been capitalized as single-electron oxidants that could oxidize some intractable reactants under photochemical conditions (MeOH, E_{red}>+3.0 V; Cl⁻, E_{red}>+2.0 V vs standard calomel electrode (SCE)), albeit requiring energetic ultraviolet photons and restricting the reaction scope only in quinoline functionalization.

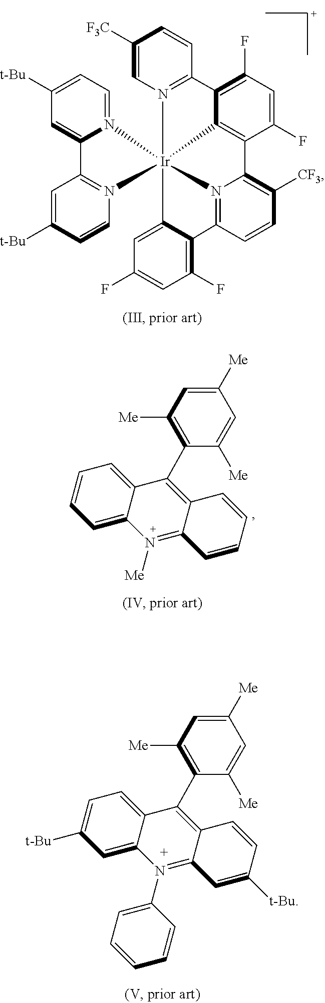

For the present organophotoredox catalyst design, without wishing to be bound by theory, the C2 and/or C4 positions of quinoline skeletons were engineered with π-extended substituents. This advantageous modification moved the absorption of the organophotoredox catalyst to the visible light region and simultaneously blocked their radicophilic sites. The present inventors have found that a simple protonation of the organophotoredox catalyst can exert an effect at least equal to other known alkylation photocatalysts. The organophotoredox catalyst of the present disclosure has a convenient and tunable activation mode that considerably simplifies its synthesis since the exocyclic N-substituents of above-noted counterparts were tethered via nucleophilic displacement or metal-catalyzed cross-couplings. Furthermore, pairing the organophotoredox catalyst with a radical precursor with reasonably low reduction potential improves the current protocols for oxidative Minisci alkylation. To this end, potassium alkyltrifluoroborates (R-BF₃K), was tested in the present example. R—BF₃K is structurally diverse, shelf-stable, and a good candidate for evaluating the organophotoredox catalyst of the present disclosure.

Solvents used in the present example were dried over 4 Å molecular sieves (beads, 8-12 mesh) and degassed by purging with argon for 30 min. The 4 Å molecular sieves were purchased from Sigma-Aldrich chemical company and were freshly activated in the oven for 12 h at 380° C. before use. Reagents were purchased from Sigma-Aldrich, Combi-Blocks, TCI America, Oakwood, and Fisher Scientific chemical companies and were used without further purification unless otherwise specified.

Nuclear magnetic resonance (NMR) spectra, including ¹H NMR, ¹³C NMR, and ¹⁹F NMR, were recorded on Bruker 500 MHz spectrometers, using the deuterium lock signal to reference the spectra. The solvent residual peaks, e.g., chloroform (CDCl3: δ 7.28 ppm and δ 77.02 ppm), were used as references. All NMR spectra were recorded at room temperature. Gas chromatography-mass spectroscopy (GC-MS) was obtained from the Agilent gas chromatography-mass spectroscopy system with helium (He) as the carrier gas. High-resolution mass spectrometry (HRMS) lifetime was measured by time-correlated single-photon counting (TCSPC), and the decay data was collected on a time-resolved emission spectrometer setup (Fluotime 200) suited with a TCSPC module (PicoHarp 300) (Picoquant GMBH) with time-resolved fluorescence decay and time-resolved anisotropy decay capabilities, monochromator, operated with symphotime software (Picoquant). Electrochemical experiments were performed with HEKA PG 340 potentiostat with Ag/AgCl as the reference electrode. The working electrode was made of glassy carbon, and a Pt wire was used as the counter electrode to complete the electrochemical setup. A scan rate of 20 mV/s was used for all experiments. All the potentials were noted with respect to the Ag/AgCl electrode unless otherwise specified. The reduction potential referenced to the standard calomel electrode (SCE) could be calculated by subtracting 0.039 V from the E(Ag/AgCl). It followed that E(SCE)=E(Ag/AgCl)−0.039 V. Electron paramagnetic resonance (EPR) was performed on a Bruker Elexsys E580 X-band EPR Spectrometer. Gas chromatography-thermal conductivity detector (GC-TCD) was conducted on an Agilent 6890N Network Gas Chromatograph for hydrogen gas (H2) analysis using argon (Ar) as the carrier gas.

Reactions were stirred magnetically unless otherwise specified. Column chromatography was performed with E. Merck silica gel 60 (230-400 mesh). Experiments were conducted in sealed 10 mL pyrextubes. Experiments under light irradiation were performed using a low-pressure 300 W Xe lamp (from Atlas Specialty Lighting, with a PE300BF light bulb from Excelitas) equipped with a water bath (Chemglass Jacketed Beaker, GC-1107-12) for efficient temperature maintenance, and all the reactions were conducted under an inert atmosphere in sealed tubes unless otherwise noted. Quantum yield was measured with a 390 nm PR160L Kessil lamp.

To establish a proof of concept, slightly excessive potassium cyclohexyltrifluoroborate (2a, Cy-BF₃K, E_{red}=+1.5 V vs SCE) was opted to alkylate lepidine (1a) in the presence of trifluoroacetic acid (TFA), chloro(pyridine)bis(dimethylglyoximato)cobalt (III) ([Co(dmgH)₂(py)]Cl) (Co, formula VI) and a quinolone photocatalyst (QN) in dioxane under visible light irradiation (Scheme 1). (Cy=cyclohexyl)

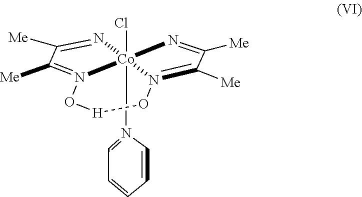

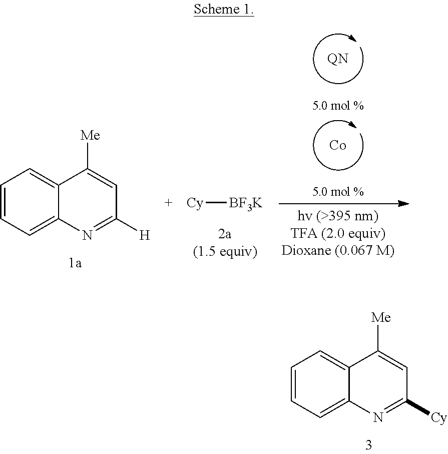

The quinoline photocatalyst tested as well as the results for each are summarized in Table 1. The yield was determined by nuclear magnetic resonance (NMR).

TABLE 1
Quinoline photocatalyst results

| Name of QN | | Yield of |
|---|---|---|
| compound | Structure of QN catalyst compound | compound 3 |
| DPQN | 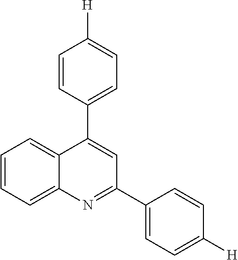 [SMILES: c1ccc(-c2cc(-c3ccccc3)c3ccccc3n2)cc1] | 25% |
| DPQN^{2-CF3} | 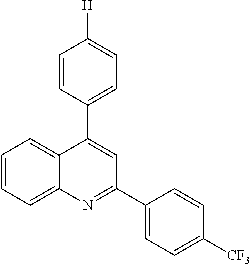 [SMILES: FC(F)(F)c1ccc(-c2cc(-c3ccccc3)c3ccccc3n2)cc1] | 19% |
| DPQN^{2-Me} | 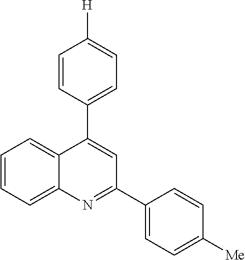 [SMILES: Cc1ccc(-c2cc(-c3ccccc3)c3ccccc3n2)cc1] | 65% |
| DPQN^{2-OMe} | 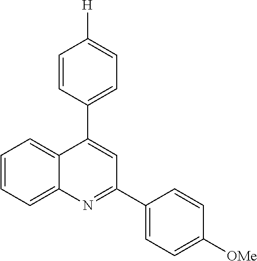 [SMILES: COc1ccc(-c2cc(-c3ccccc3)c3ccccc3n2)cc1] | 79% |
| DPQN^{4-OMe} | 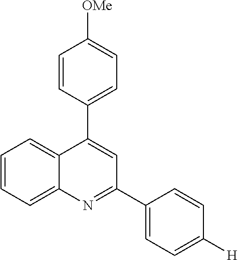 [SMILES: COc1ccc(-c2cc(-c3ccccc3)nc3ccccc23)cc1] | 86% |
| DPQN^{2,4-di-OMe} | 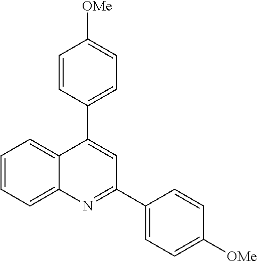 [SMILES: COc1ccc(-c2cc(-c3ccc(OC)cc3)c3ccccc3n2)cc1] | 96% |
| QN^{2-Ph} | 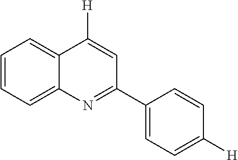 [SMILES: c1ccc(-c2ccc3ccccc3n2)cc1] | 0% |
| QN^{4-Ph} | 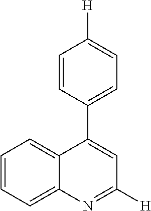 [SMILES: c1ccc(-c2ccnc3ccccc23)cc1] | 0% |
| QN^{2-Ph-4-Me} | 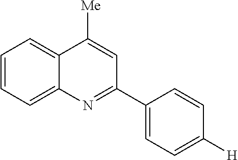 [SMILES: Cc1cc(-c2ccccc2)nc2ccccc12] | 0% |
| QN^{2-Me-4-Ph} | 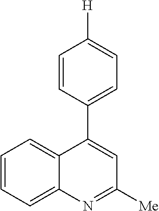 [SMILES: Cc1cc(-c2ccccc2)c2ccccc2n1] | 0% |

The reactions were performed with compound 1a (0.10 mmol, 1.0 equiv), compound 2a (0.15 mmol, 1.5 equiv), QN (5.0 μmol, 5.0 mol %), [Co(dmgH)₂(py)]Cl (5.0 μmol, 5.0 mol %), TFA (0.20 mmol, 2.0 equiv) in dioxane (1.5 mL, 0.067 M) under N₂ at about 37° C. and irradiated by >395 nm light for 20 hours.

As set out in Table 1, DPQN^{2,4-di-OMe}, DPQN^{4-OMe}, DPQN^{2-OMe}, and DPQN^{2-Me} showed good results with yields of at least 65% whereas the remaining compounds tested all had an inadequate yield of 25% or less. DPQN^{2,4-di-OMe} had the best yield at 96% and was further tested by reducing the loading concentration from 5.0 mol % to 0.025 mol %. The yield obtained with the loading concentration of 0.025 mol % of DPQN^{2,4-di-OMe} was 84%. Because of the instrumental role of cationization for enhancing the photocatalytic performance, electron-donating groups are beneficial. Furthermore, locating the electron-releasing substituents on DPQN could structurally correlate with the donor-acceptor patterns of acridiniums. Unsurprisingly, the yield of compound 3 dropped when an electron-withdrawing group (—CF₃) resided on the DPQN parent structure. On the contrary, the productivity was significantly elevated when using methylated and methoxylated DPQNs (i.e. DPQN^{2,4-di-OMe}, DPQN^{4-OMe}, DPQN^{2-OMe}, and DPQN^{2-Me}). Without wishing to be bound by theory, -Me and —OMe combat the susceptibility of catalysts towards radical attack, therefore conferring stability against their deactivation. This can therefore explain why DPQN^{2,4-di-OMe} ranked as the most robust and efficient photocatalyst in the series tested (Table 1), giving a very high yield of the cyclohexylation product even at 0.025 mol % loading, albeit for a longer reaction time (72 h).

Removal of either aryl handle from DPQNs completely suppressed the reaction, presumably due to the unmatched photoabsorptive profiles or the non-productive consumption of radical intermediates. Control experiments showed that photocatalyst, cobalt, acid, light and inert atmosphere were important for this photoinduced oxidative cross-coupling reaction. Other boron reagents were ineffective, which might attribute to their prohibitive oxidation potentials (E_{red}>+2.5 V vs SCE for cyclohexylboronic acid, Cy-B(OH)₂ and its pinacol ester, Cy-Bpin). Notably, among some commercial photocatalysts evaluated, [Ru(bpy)₃](PF₆)₂, Eosin Y, Rose bengal, and Rhodamine 6G brought poor results of the Minisci alkylation (0%, 25%, 0% and 0% yields respectively).

Further experimentation according Scheme 1 was performed by varying different conditions as detailed in Table 2 below.

TABLE 2
Experimental conditions and results

| QN | Co | Acid (equiv) | Solvent (mL) | Yield (%) |
|---|---|---|---|---|
| DPQN | [Co(dmgH)₂(py)]Cl | TFA (3.0) | Dioxane (1.0) | 59 |
| DPQN^{2-CF3} | [Co(dmgH)₂(py)]Cl | TFA (3.0) | Dioxane (1.0) | 16 |
| DPQN^{2-Me} | [Co(dmgH)₂(py)]Cl | TFA (3.0) | Dioxane (1.0) | 63 |
| DPQN^{2-OMe} | [Co(dmgH)₂(py)]Cl | TFA (3.0) | Dioxane (1.0) | 68 |
| DPQN^{4-OMe} | [Co(dmgH)₂(py)]Cl | TFA (3.0) | Dioxane (1.0) | 67 |
| DPQN^{2,4-di-OMe} | [Co(dmgH)₂(py)]Cl | TFA (3.0) | Dioxane (1.0) | 73 |
| DPQN^{2,4-di-OMe} | [Co(dmgH)₂(py)]Cl | TFA (3.0) | Dioxane (1.5) | 93 |
| DPQN^{2,4-di-OMe} | [Co(dmgH)₂(py)]Cl | TFA (2.0) | Dioxane (1.5) | 96 |
| DPQN^{2,4-di-OMe} | [Co(dmgH)₂(py)]Cl | TFA (1.0) | Dioxane (1.5) | <5 |
| DPQN^{2,4-di-OMe} | [Co(dmgH)₂(py)]Cl | — | Dioxane (1.5) | 0 |
| DPQN^{2,4-di-OMe} | [Co(dmgH)₂(py)]Cl | TFA (2.0) | MeCN (1.5) | 51 |
| DPQN^{2,4-di-OMe} | [Co(dmgH)₂(py)]Cl | TFA (2.0) | EtOAc | 86 |
| DPQN^{2,4-di-OMe} | [Co(dmgH)₂(py)]Cl | TFA (2.0) | Dioxane (1.5) | <5 |
| (under air) | | | | |
| DPQN^{2,4-di-OMe} | [Co(dmgH)₂(py)]Cl | TFA (2.0) | Dioxane (1.5) | 0 |
| (at room | | | | |
| temperature in | | | | |
| the dark) | | | | |
| DPQN^{2,4-di-OMe} | [Co(dmgH)₂(py)]Cl | TFA (2.0) | Dioxane (1.5) | 0 |
| (at 80° C. in the | | | | |
| dark) | | | | |
| — | [Co(dmgH)₂(py)]Cl | TFA (2.0) | Dioxane (1.5) | 0 |
| DPQN^{2,4-di-OMe} | — | TFA (2.0) | Dioxane (1.5) | 0 |
| DPQN^{2,4-di-OMe} | [Co(dmgH)₂(py)]Cl | HCl (2.0) | Dioxane (1.5) | 30 |
| DPQN^{2,4-di-OMe} | [Co(dmgH)₂(py)]Cl | AcOH (2.0) | Dioxane (1.5) | 0 |
| DPQN^{2,4-di-OMe} | CoCl₂ | TFA (2.0) | Dioxane (1.5) | <5 |
| DPQN^{2,4-di-OMe} | Co(dmgBF₂)₂ | TFA (2.0) | Dioxane (1.5) | 82 |

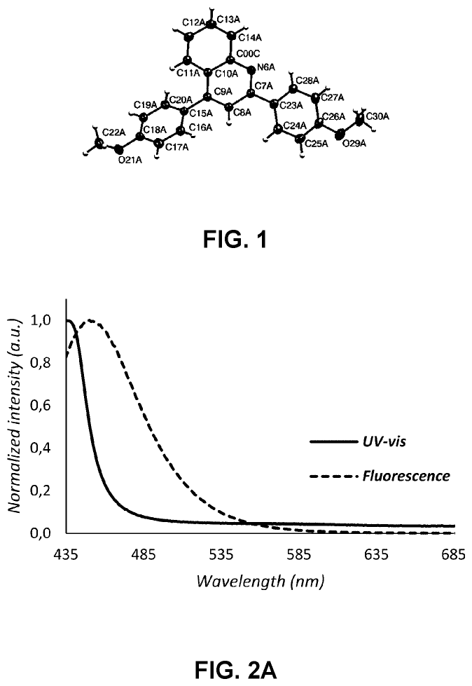

Therefore, 1.0 equiv heteroarene, 1.5 equiv R-BF₃K, 5.0 mol % DPQN^{2,4-di-OMe}, 5.0 mol % [Co(dmgH)₂(py)]Cl and 2.0 equiv TEA in dioxane (0.067 M) under argon with visible light irradiation (>395 nm) were the preferred conditions. The synthesis of DPQN^{2,4-di-OMe} was explored and it was found that DPQN^{2,4-di-OMe} can be prepared in a multigram scale (56%, 2.9 g) via the facile aldehyde-alkyne-amine (A³) couplings with a Lewis acid (LA) (scheme 2). Its structure was unambiguously confirmed by X-ray crystallography (FIG. 1). The reactions were performed with amine (15.0 mmol, 1.0 equiv), aldehyde (15.0 mmol, 1.0 equiv), MgSO₄ (7.5 mmol, 0.5 equiv) in DCM (5.0 mL, 3.0 M) at room temperature for 2.0 hours; then alkyne (22.5 mmol, 1.5 equiv), Fe(OTO₃ (0.375 mmol, 2.5 mol %), AcOH (22.5 mmol, 1.5 equiv) in toluene (15 mL, 1.0 M) at 140° C. for 16 hours.

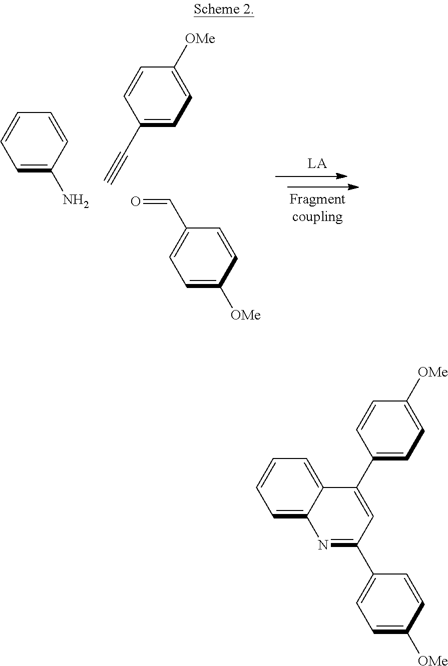

An advantage of DPQN^{2,4-di-OMe} is its reduced cost compared to current commercial catalysts. Table 3 below details the price of the chemicals to synthesize DPQN^{2,4-di-OMe}. Based on Table 3, the cost for 2.92 g of DPQN^{2,4-di-OMe} could be estimated to be $212 CAD, and its unit price would be $7.3 CAD/100 mg, which is significantly lower than the acridinium catalyst ($145 CAD/100 mg from Sigma Aldrich and 4CzIPN $762 CAD/100 mg from Sigma Aldrich).

TABLE 3
Cost summary for DPQN^{2,4-di-OMe} synthesis

| | Unit price (CAD) from | | | |
|---|---|---|---|---|
| Chemical | Sigma Aldrich | Quantity | | Cost (CAD) |
| 4-anisaldehyde | 0.21$/g | 2.0 | g | $0.42 |
| Aniline | 0.035$/g | 1.4 | g | $0.049 |
| 4-ethynylanisole | 67$/g | 3.0 | g | $201 |
| MgSO₄ | 0.043$/g | 0.90 | g | $0.039 |
| Fe(OTf)₃ | 52$/g | 0.18 | g | $9.3 |
| AcOH | 0.025$/mL | 1.3 | mL | $0.033 |
| CH₂Cl₂ | 0.11$/mL | 5.0 | mL | $0.55 |
| Toluene | 0.020$/mL | 15 | mL | $0.30 |

Furthermore, in terms of the catalyst synthesis, the preparation of DPQN^{2,4-di-OMe} photocatalyst is advantageous because of a shorter synthetic time length and using reagents that are easy to handle. In general, the synthesis of acridinium catalysts involves multiple steps for a long reaction time, in which the N-functionalization is realized by nucleophilic substitution or metal-catalyzed cross-coupling. In addition, the synthesis is often accomplished by Grignard reactions. In contrast, in the present example a two-step aldehyde-alkyne-amine coupling reaction was designed for the diarylquinoline catalyst preparation, wherein the starting materials are readily available and convenient to handle. Furthermore, N-substitution is unnecessary in the procedure since the catalyst could be easily activated under typical Minisci acidic conditions.

Henceforth in the present example, DPQN^{2,4-di-OMe} with chloro(pyridine)bis(dimethylglyoximato)cobalt (III) was used as the catalyst system unless otherwise specified. This catalyst system was first used to investigate the alkylation of lepidine 1a with various R-BF₃K (Table 4). The reaction conditions were 4-Me-DPQN (0.10 mmol, 1.0 equiv), potassium alkyltrifluoroborate (R-BF₃K, 0.15 mmol, 1.5 equiv), DPQN^{2,4-di-OMe} (5.0 μmol, 5.0 mol %), [Co(dmgH)₂(py)]Cl (5.0 μmol, 5.0 mmol %), and TFA (0.20 mmol, 2.0 equiv) in dioxane (1.5 mL, 0.067 M) under light irradiated at ˜37° C. for 20 h under N₂. Yields in the table refer to the isolated yields unless otherwise specified. For compound 6, ethyl acetate (EtOAc) was used as the solvent. For compound 17, 3.0 equiv R-BF₃K was used.

A broad spectrum of R-BF₃K, including 1°, 2° and 3° ones, were proven viable in this transformation. Simple alkyl groups such as the isopropyl, sec-butyl, n-pentyl, and tert-butyl could be installed, providing the elaborated lepidines smoothly (compounds 4 to 7), so as the four to six-membered cyclic substituents (compounds 8 and 9). The bridged reagents like 1-adamantyl and 2-norbonyl ones were heteroarylated successfully, which afforded the target products compounds 10 and 11 in good to excellent yields. Functionalized alkyltrifluoroborates bearing ester, ketone, ethereal, carbamoyl, benzyloxy, allyloxy, and propargyloxy groups were also compatible, and the lepidine was decorated in satisfactory yield (compounds 12 to 22).

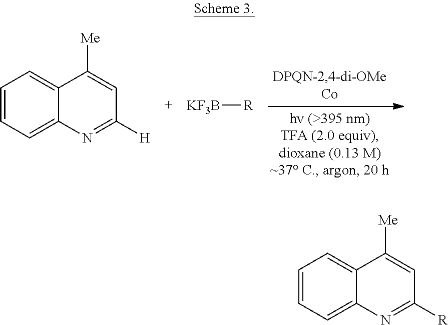

TABLE 4
Alkylation of lepidine

| Compound | | |
|---|---|---|
| number | Compound product obtained | Yield |
| 4 | 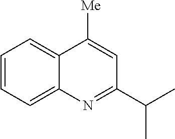 | 83% |
| 5 | 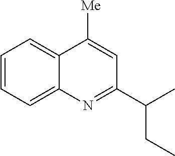 | 70% |
| 6 | 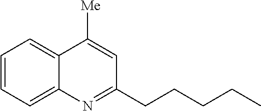 [SMILES: CCCCCc1cc(C)c2ccccc2n1] | 32% |
| 7 | 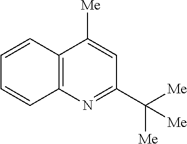 [SMILES: Cc1cc(C(C)(C)C)nc2ccccc12] | 70% |
| 8 | 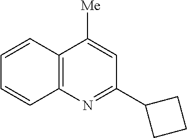 [SMILES: Cc1cc(C2CCC2)nc2ccccc12] | 27% |
| 9 | 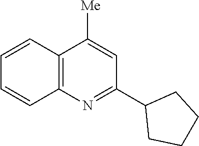 [SMILES: Cc1cc(C2CCCC2)nc2ccccc12] | 86% |
| 10 | 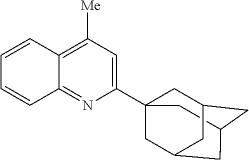 [SMILES: Cc1cc(C23CC4CC(CC(C4)C2)C3)nc2ccccc12] | 93% |
| 11 | 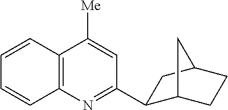 [SMILES: Cc1cc(C2CC3CCC2C3)nc2ccccc12] | 72% |
| 12 | 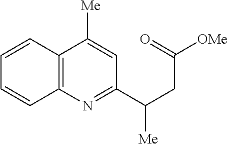 [SMILES: COC(=O)CC(C)c1cc(C)c2ccccc2n1] | 70% |
| 13 | 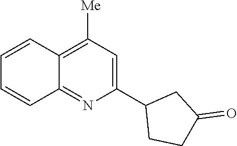 [SMILES: Cc1cc(C2CCC(=O)C2)nc2ccccc12] | 62% |
| 14 | 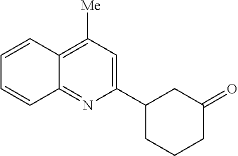 [SMILES: Cc1cc(C2CCCC(=O)C2)nc2ccccc12] | 76% |
| 15 | 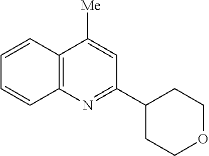 [SMILES: Cc1cc(C2CCOCC2)nc2ccccc12] | 91% |
| 16 | 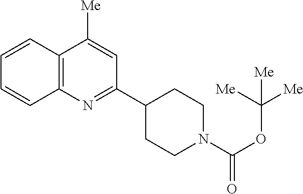 [SMILES: Cc1cc(C2CCN(C(=O)OC(C)(C)C)CC2)nc2ccccc12] | 61% |
| 17 | 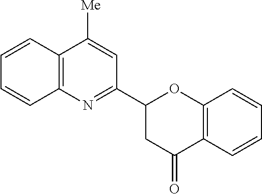 [SMILES: Cc1cc(C2CC(=O)c3ccccc3O2)nc2ccccc12] | 53% |
| 18 | 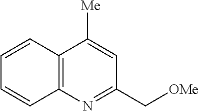 [SMILES: COCc1cc(C)c2ccccc2n1] | 59% |
| 19 | 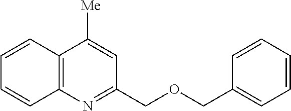 [SMILES: Cc1cc(COCc2ccccc2)nc2ccccc12] | 59% |
| 20 | 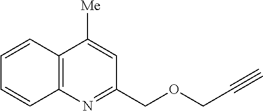 [SMILES: C#CCOCc1cc(C)c2ccccc2n1] | 54% |
| 21 | 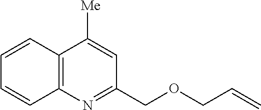 [SMILES: C=CCOCc1cc(C)c2ccccc2n1] | 36% |
| 22 | 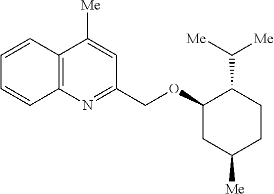 [SMILES: Cc1cc(CO[C@@H]2C[C@H](C)CC[C@H]2C(C)C)nc2ccccc12] | 57% |

Further scope examination with different heteroaromatic pharmacophores with Cy-BF₃K (compound 2a) as the coupling partner was conducted (Scheme 4). Unless otherwise specified the reaction conditions were: heteroarene (Het-H, 0.10 mmol, 1.0 equiv), potassium alkyltrifluoroborate (R-BF₃K, 0.15 mmol, 1.5 equiv), DPQN^{2,4-di-OMe} (5.0 μmol, 5.0 mol %), [Co(dmgH)₂(py)]Cl (5.0 μmol, 5.0 mmol %), and TFA (0.20 mmol, 2.0 equiv) in dioxane (1.5 mL, 0.067 M) under light irradiated at ˜37° C. for 20 h under N₂. For compounds 24, 28, 33, 34, 35, and 39-43 3.0 equiv R-BF₃K was used. For compounds 29, 36, and 37 4.0 equiv R—BF₃K was used. For compounds 33-37 and 42 3.0 equiv TFA was used. For compound 29 EtOAc was the solvent and the reaction was run for 40 h. For compound 29 a ratio Mono:di=10:1 was obtained where mono is a C2 alkylation and di is both a C2 and C4 alkylation. For compound 30 a ratio C1:C3=6.2:1 was obtained where C1 is the alkylation at C1 and C3 is the alkylation at C3. For compound 37 the yield was determined by NMR. As shown in Table 5, a variety of substituents on heterocycles like cyano, halo, ketone, alkoxy, ester, sulfonamido, amino, amido groups and others were well tolerated in this reaction (23 to 52). Other than quinoline compounds, elaboration of isoquinoline, pyridine, bipyridine, phenanthroline, phenanthridine, benzimidazole, benzothiazole, thiazole, quinoxalinone and quinazolinone were shown to be effective (compounds 30 to 43). Yields in the tables below refer to the isolated yields unless otherwise specified.

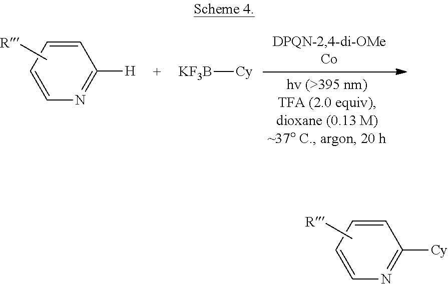

TABLE 5
Alkylation of heteroarenes

| Compound | | |
|---|---|---|
| number | Compound structure | Yield |
| 23 | 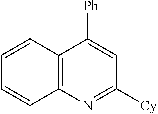 | 80% |
| 24 | 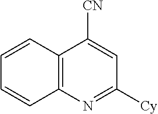 | 56% |
| 25 | 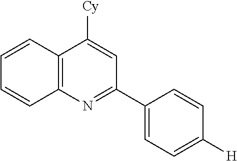 | 66% |
| 26 | 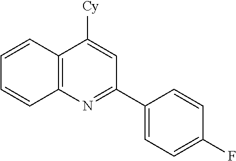 | 53% |
| 27 | 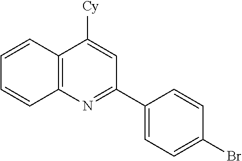 | 74% |
| 28 | 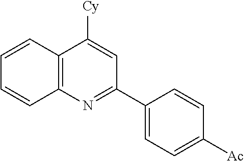 | 43% |
| 29 | 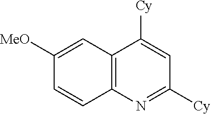 | 43% |
| 30 | 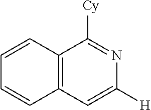 | 63% |
| 31 | 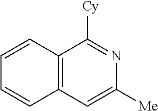 | 70% |
| 32 | 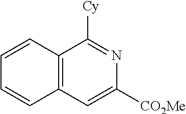 | 65% |
| 33 | 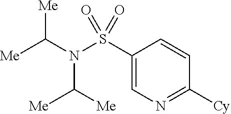 | 41% |
| 34 | 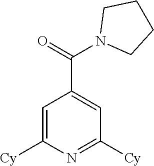 | 32% |
| 35 | 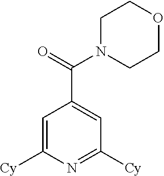 | 42% |
| 36 | 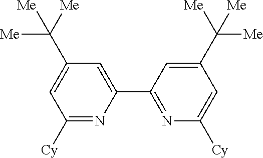 | 28% |
| 37 | 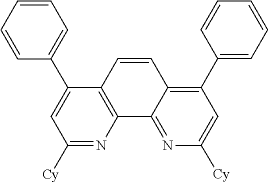 | 45% |
| 38 | 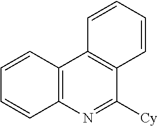 | 96% |
| 39 | 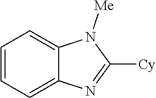 | 57% |
| 40 | 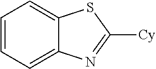 | 60% |
| 41 | 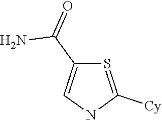 | 45% |
| 42 | 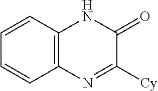 | 58% |
| 43 | 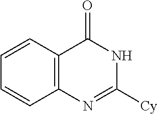 | 73% |

To showcase the robustness of the heteroarene functionalization method of the present disclosure, the alkylation (with Cy-BF₃K) of substrates with high molecular complexity was evaluated (Scheme 4, Table 6). Unless otherwise specified the reaction conditions: heteroarene (Het-H, 0.10 mmol, 1.0 equiv), potassium alkyltrifluoroborate (R—BF₃K, 0.15 mmol, 1.5 equiv), DPQN^{2,4-di-OMe} (5.0 μmol, 5.0 mol %), [Co(dmgH)z(py)]Cl (5.0 μmol, 5.0 mmol %), and TEA (0.20 mmol, 2.0 equiv) in dioxane (1.5 mL, 0.067 M) under light irradiated at ˜37° C. for 20 h under N₂. For compounds 44-47, 51 and 52 3.0 equiv R—BF₃K was used. For compounds 44, 45, 47, 48, 51 and 52 3.0 equiv TEA was used. For compound 50 a ratio Mono:di=1:1 was obtained and for compound 52 a ratio Mono:di=2:1 was obtained where mono is only a C2 alkylation and di is a C2 and C6 alkylation.

Encouragingly, cyclohexylation of dichloropurine provided the expected product 44 in moderate yield. Couplings of pyridines consisting of alanine, pyrrolidine, and menthol moieties proceeded efficiently (compounds 45, 46, and 50). The more structurally complex pyridine derivatives were also successfully applied in the present protocol. For example, loratadine and roflumilast, which were registered for allergy medications and phosphodiesterase-4 (PED-4) inhibition, respectively, were transformed into the desirable products with their carbamate or amide group remained untouched (compounds 51 and 52). Other bioactive examples including the antifungal agent voriconazole and the marketed isoquinoline-based vasodilator, fasudil, were utilized directly without functional group protection (compounds 47 and 48). Finally, cinchonine, which is quinoline-cored and bears both hydroxyl and amino groups, was easily modified the present protocol (compound 49).

TABLE 6
Complex substrates results

| Compound | | |
|---|---|---|
| number | Compound structure | Yield |
| 44 | 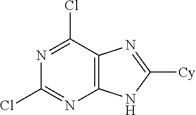 | 64% |
| 45 | 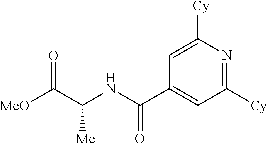 | 40% |
| 46 | 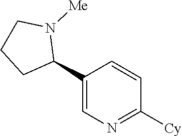 | 44% |
| 47 | 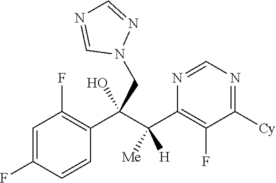 | 39% |
| 48 | 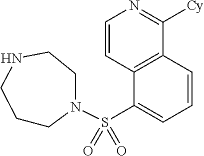 | 65% |
| 49 | 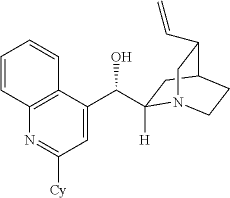 | 44% |
| 50 | 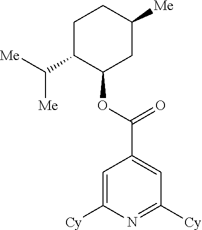 | 50% |
| 51 | 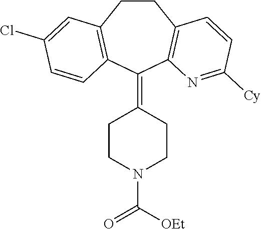 | 46% |
| 52 | 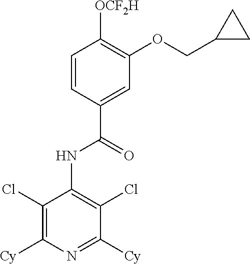 | 86% |

DPQN^{2,4-di-OMe} was characterized by several spectroscopic techniques to collect some PP of its photophysical parameters. Five formulated solutions were prepared with degassed dioxane in 10 mL volumetric flasks. For flask A, DPQN^{2,4-di-OMe} (17.1 mg, 0.05 mmol) and TEA (3.8 μL, 0.05 mmol) were added; for flask B, DPQN^{2,4-di-OMe} (17.1 mg, 0.050 mmol) was added; for flask C, DPQN (14.1 mg, 0.050 mmol) and TEA (3.8 μL, 0.050 mmol) were added; for flask D, DPQN^{2-CF3} (17.5 mg, 0.050 mmol) and TEA (3.8 μL, 0.050 mmol) were added; for the flask E, potassium cyclohexyltrifluorobo-rate (2a, 47.5 mg, 0.25 mmol) and tetrabutylammonium tetrafluoroborate (Bu4NBF4, 82.3 mg, 0.25 mmol) were added. All these flasks were diluted to 10 mL to set the concentration to be 5.0 mM, 5.0 mM, 5.0 mM, 5.0 mM, and 25.0 mM, respectively.

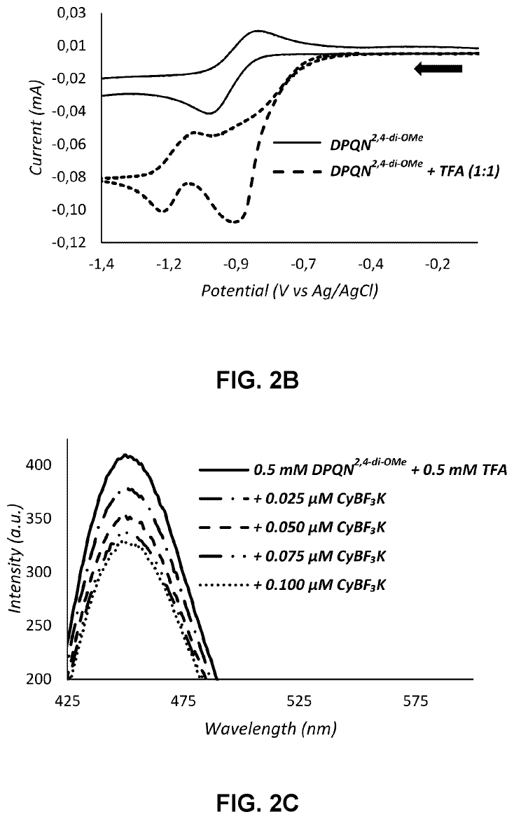

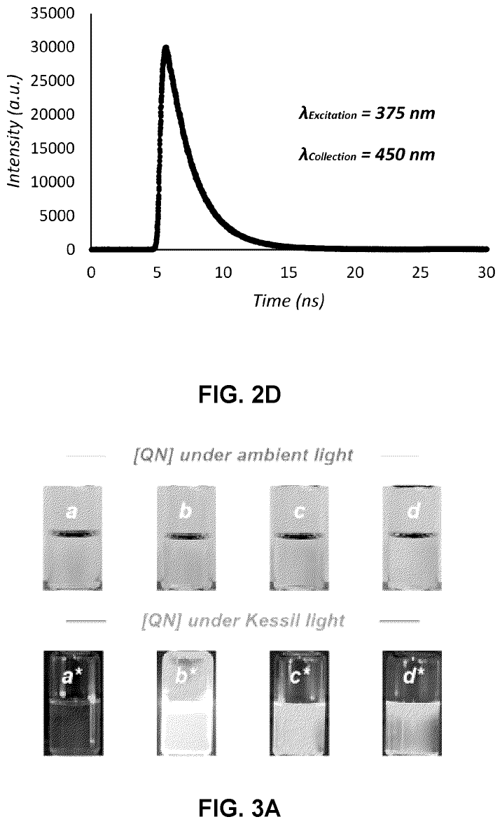

UV-vis and fluorescence spectra demonstrated that the positively charged DPQN^{2,4-di-OMe} absorbed strongly above 395 nm and emitted mostly at around 455 nm, with the intersection at 441 nm (FIGS. 2A, 2C, and 2D). The excited-state redox potential E_{1/2}(PC*/PC−) was estimated by the following equation

$${E}_{1 / 2} ( {PC}^{*} / {PC}^{-} ) = {E}_{0 - 0} + {E}_{1 / 2} ( PC / {PC}^{-} )$$

where E^{1/2}(PC/PC⁻) was the ground state redox potential; E0-0 was the energy difference between 0th vibrational states of the ground state and excited state, which can be approximated by the intersection point between the normalized absorption and emission spectra. Since DPQN^{2,4-di-OMe} gave irreversible peaks in cyclic voltammogram, E_{p/2} (PC/PC⁻) was used for its ground state redox potential, E1/2 (PC/PC−), which was determined to be −0.81 V. For the excitation energy, E0-0, since the wavelength of the cross point in absorption and emission spectra was 441 nm, it could be translated into E0-0=2.81 eV.

$${E}_{1 / 2} ( {PC}^{*} / {PC}^{-} ) = {E}_{0 - 0} + E 1 / 2 ( PC / {PC}^{-} ) = 2.81 V - 0.81 V = + 2 \text{.00} V vs Ag / AgCl$$

$$E 1 / 2 ( {PC}^{*} / PC - ) = 2. V - 0.039 V = + 1.96 V vs SCE$$

A quartz cuvette (1.0 cm×1.0 cm×3.5 cm) was added 0.20 mL of the 5.0 mM solution from flask A and was diluted to 2.0 mL with dioxane as a 0.50 mM solution, which was then irradiated at 395 nm. Duplicate experiments were performed with the addition of 2.0, 4.0, 6.0, 8.0 μL 25 mM solution from flask E before being diluted to 2.0 mL. The resulting stacked UV-vis fluorescence emission spectra is shown in FIG. 2A.

Cyclic voltammogram (CV) showed that the redox processes of neutral DPQN^{2,4-di-OMe} was electrochemically reversible (E_{1/2}([QN]/[QN⁻])=−0.95 V vs SCE), while it was irreversibly reduced in the presence of TFA (E_{p/2}([QN-H⁺]/[QN-H⁻]=−0.81 V vs SCE) (FIG. 2B). Without wishing to be bound by theory, such changes make the catalyst more prone to reduction upon protonation which in part justifies the requirement of acid in the present process. Simple calculations uncovered a long-lived excited state of DPQN^{2,4-di-OMe} in protonated form (τ_{f}=2.0 ns and E_{1/2}([QN-H⁺]*/[QN-H*])=+1.96 V vs SCE). With such an extensive oxidation window, photoinduced electron transfer (PET) with R—BF₃K to engender the alkyl radicals was assured, which was also evidenced by the Stern-Volmer plot (K_{SV}=2.5 mM⁻¹).

A quartz cuvette (1.0 cm×1.0 cm×3.5 cm) was added 0.20 mL of the 5.0 mM solution from flask A and was diluted to 2.0 mL with dioxane as a 0.50 mM solution, which was then irradiated at 395 nm. Duplicate experiments were performed with the addition of 2.0, 4.0, 6.0, 8.0 μL 25 mM solution from flask E before being diluted to 2.0 mL. The resulting fluorescence emission spectra is shown in FIG. 2C.

A quartz cuvette (1.0 cm×1.0 cm×3.5 cm) was filled with 0.20 of the 5.0 mM solutions from flasks A and diluted to 2.0 mL with dioxane as a 0.5 mM solution, which was then submitted to the fluorescence lifetime spectrometer for the experiment. The solution was excited at 375 nm, and the photon counts were recorded at 450 nm. Monoexponentially fitting trend line gave the lifetime τ=2.07±0.01 ns, as shown in FIG. 2D.

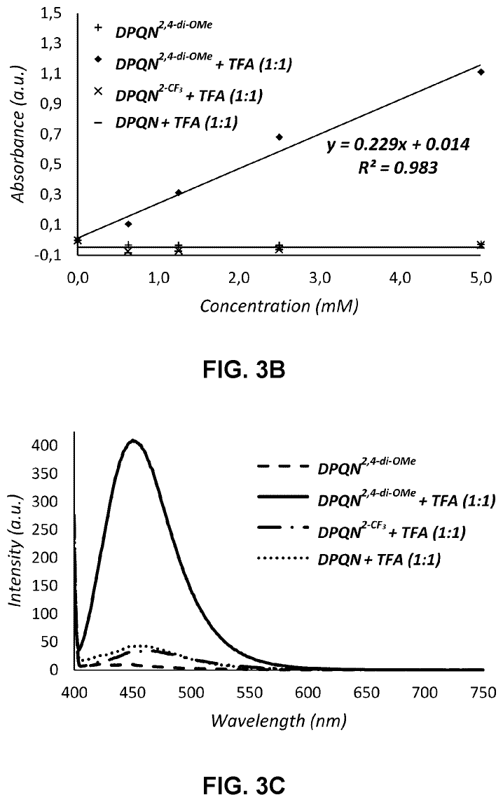

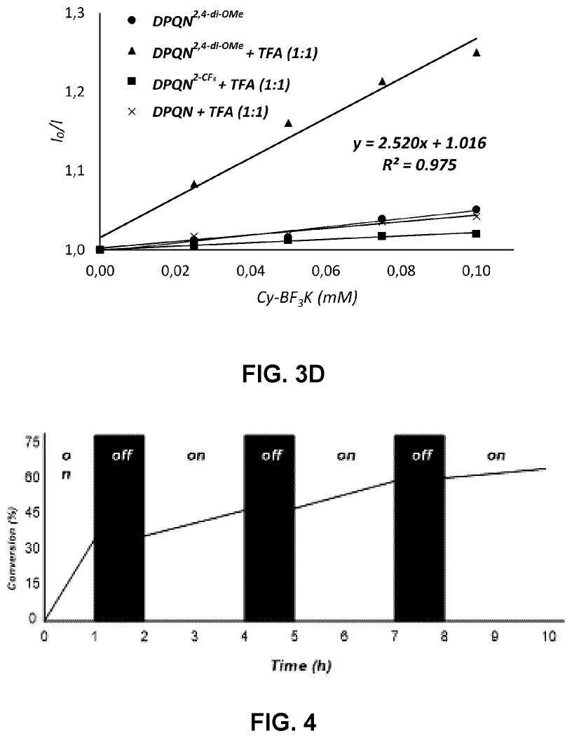

Secondly, to rationalize the advantageous effect of methoxy substituents on the protonated DPQN^{2,4-di-OMe} and further elucidate the “proton activation” concept, the electronically neutral and deficient variants (DPQN and DPQN^{2-CF3}) as well as the non-protonated form of DPQN^{2,4-di-OMe} were selected as representative catalysts for more investigations. Interestingly, the stronger visible light absorption of protonated DPQN^{2,4-di-OMe} could be directly visualized under ambient conditions as its neutral form and the other two in acidic media were basically colorless. Such differences were even more obvious under light irradiation since proton-activated DPQN^{2,4-di-OMe} gave a much brighter luminescence (FIG. 3A). With this observation in mind, the absorptivity and fluorescence of these DPQNs were measured. Protonated DPQN^{2,4-di-OMe} outweighed the other three in both measurements, which agreed with its markedly higher Stern-Volmer quenching efficiency by Cy-BF₃K (FIGS. 3B, 3C, and 3D).

A quartz cuvette (1.0 cm×1.0 cm×3.5 cm) was added 2.0 mL of the abovementioned 5.0 mM solutions from flasks A and successively diluted to 2.5 mM, 1.25 mM, and 0.625 mM with dioxane to perform UV-vis experiments. Duplicated experiments were performed with solutions from flasks B to D, and the absorptions of different catalytic solutions (DPQN^{2,4-di-OMe}, DPQN^{2,4-di-OMe}+TFA at 1:1, DPQN^{2-CF3}+TFA at 1:1, and DPQN+TFA at 1:1) at 395 nm were plotted, as shown in FIG. 3B.

A quartz cuvette (1.0 cm×1.0 cm×3.5 cm) was added 2.0 mL of the abovementioned 5.0 mM solutions from flasks A and successively diluted to 2.5 mM, 1.25 mM, and 0.625 mM with dioxane to perform UV-vis experiments. Duplicated experiments were performed with solutions from flasks B to D, and the absorptions of different catalytic solutions at 395 nm were plotted and are shown in FIG. 3B. A quartz cuvette (1.0 cm×1.0 cm×3.5 cm) was filled with 0.20 mL of the 5.0 mM solutions from flasks A and diluted to 2.0 mL with dioxane as a 0.50 mM solution, which was then irradiated at 395 nm. Duplicated experiments were performed with solutions from flasks B to D, and the resulting fluorescence spectra are shown in FIG. 3C.

While the quenching effect was observed with protonated DPQN^{2,4-di-OMe} no prominent quenching was observed with other DPQN solutions from flasks B to D. The Stern-Volmer plots of each DPQN solution are shown in FIG. 3D.

These results emphasized the significance of electron-releasing substituents on the diarylquinoline framework and the presence of an acid, which synergistically augmented the photoproductivity of DPQN^{2,4-di-OMe}.

Next, to gain insight into the overall reaction process, a light on-and-off experiment was performed. To a 10 mL pyrex microwave tube equipped with a Teflon-coated magnetic stirring bar were added heteroarene (1a, 13.3 μL, 0.10 mmol, 1.0 equiv), potassium cyclohexyltrifluoroborate (2a, 28.5 mg, 0.15 mmol, 1.5 equiv), DPQN^{2,4-di-OMe} (1.7 mg, 5.0 mmol, 5.0 mol %) and [Co(dmgH)2(py)]Cl (2.0 mg, 5.0 mmol, 5.0 mol %). The tube was sealed with a rubber septum, evacuated and backfilled with argon three times before dioxane (1.5 mL) was injected. To the mixture was then added TFA (15.3 μL, 0.20 mmol, 2.0 equiv) in the glovebox, and the tube was sealed again by an aluminum cap with a septum, which was taken out from the glovebox and stirred at ˜37° C., with or without a 300 W Xe lamp (with a 395 nm filter) irradiation, as the time period indicated in FIG. 4. At the end of each period, a small portion (˜0.20 mL) of the reaction mixture was taken by a syringe, basified with sat NaHCO₃ (aq), extracted with EtOAc, and concentrated to afford the crude sample, which was taken for ¹H NMR analysis.

The experiment indicated that continuous light irradiation was needed for the reaction since a minimal increase of product yield persisted in the dark (FIG. 4 and Table 7). In light of the quantum yield (Φ=9.7%), a chain process was less likely in the present system. When radical quenchers, 3,5-di-tert-4-butylhydroxytoluene (BHT) and 2,2,6,6-tetramethylpiperidine 1-oxyl (TEMPO), were present, the desired reactivities were mostly inhibited, and the cyclohexyl adduct compound 53 was detected in the latter case, which suggested the involvement of alkyl radicals in the reaction (Scheme 5). Also, radical-clock reagents, including (cyclopropylmethy)trifluoroborate (compound 2u) and 5-hexenyltrifluoroborate (compound 2v), were subjected to the standard conditions (Scheme 6). As expected, the ring-opening and -closing products were isolated successfully (compounds 54 and 55), again signaling the presence of radical intermediacy.

TABLE 7
Results of the light on/off experiment

| Time (h) | Light | Conversion of 1a to 3 (%) |
|---|---|---|
| 1 | On | 38 |
| 2 | Off | 38 |
| 4 | On | 49 |
| 5 | Off | 50 |
| 7 | On | 62 |
| 8 | Off | 62 |
| 10 | On | 69 |

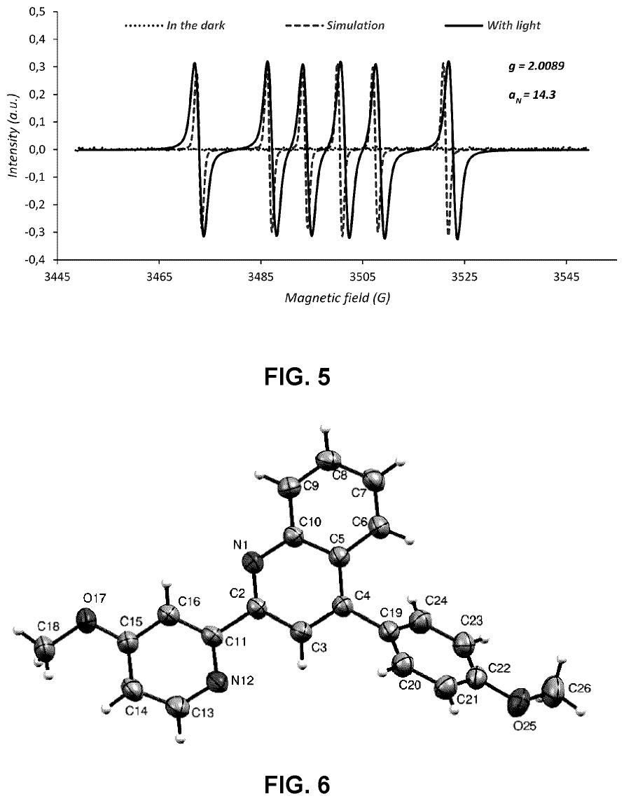

Furthermore, electron paramagnetic resonance (EPR) provided direct evidence for the existence of open-shell species. Two 10 mL pyrex microwave tubes equipped with Teflon-coated magnetic stirring bars were added 5,5-dimethyl-1-pyrroline-N-oxide (DMPO, 11.3 mg, 0.10 mmol, 1.0 equiv), potassium alkyltrifluoroborate (2a, 19 mg, 0.10 mmol, 1.0 equiv), and DPQN^{2,4-di-OMe} (34.1 mg, 0.10 mol, 1.0 equiv). The tubes were sealed with rubber septa, evacuated and backfilled with argon three times before dioxane (1.0 mL) was injected. To the mixture was then added TFA (7.7 μL, 0.10 mmol, 1.0 equiv) in the glovebox and sealed again by an aluminum cap with a septum, which was taken out from the glovebox and stirred at ˜37° C. with or without light irradiation of a 300 WXe lamp with a 395 nm filter. After 2 h, the reactions were taken for electron paramagnetic resonance (EPR) analysis. Under light irradiation, Cy. was trapped by 5,5-dimethyl-1-pyrroline-N-oxide (DMPO, compound 56), whose EPR spectrum was fully consistent with the literature and a simulation performed (compound 57), while such a response was silenced in the dark (Scheme 7, FIG. 5). Lastly, H₂ evolution was confirmed by gas chromatography-thermal conductivity detector (GC-TCD), which was in accordance with the acceptorless oxidative coupling design of Scheme 8.

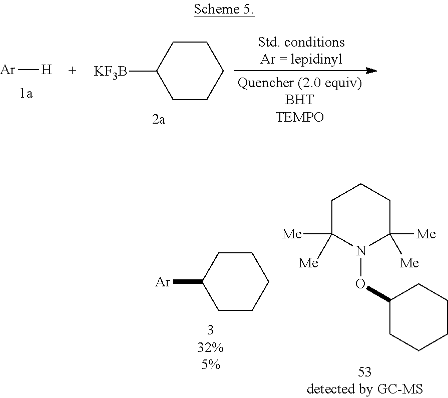

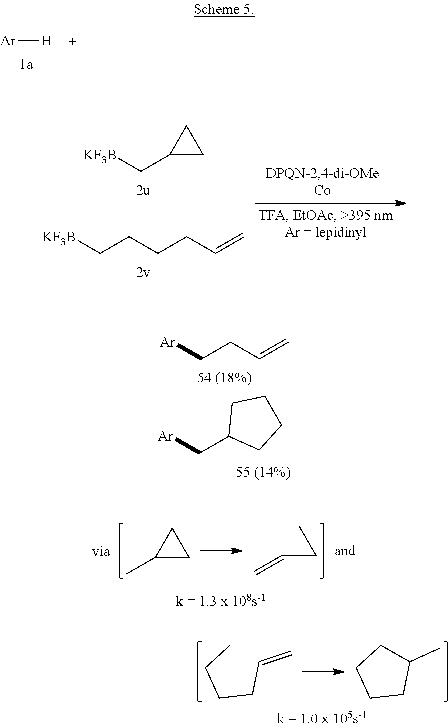

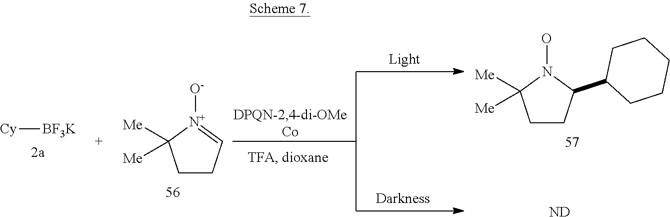

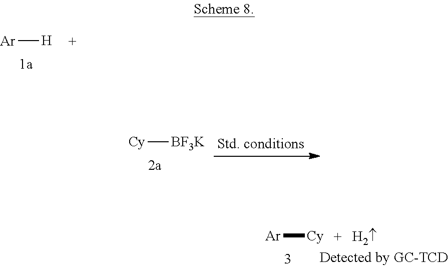

Taken together, a reaction mechanism was proposed and shown in Scheme 9. Driven by the visible light irradiation, the excited diarylquinoline, [QN-H⁺]*, underwent a reductive quenching by the R-BF₃K compound 2, generating two radical intermediates, alkyl radical I and heteroaryl radical [QN-H]*. While the former nucleophilic carbon radical I attacked the protonated heteroarene to give a radical cation III, the latter [QN-H]⁻ (E_{p/2}([QN-H⁺]/[QN-H*]=−0.81 V vs SCE) reduced the cobaloxime [Co^{III}] into [Co^{II}](E_{red}([Co^{III}]/[Co^{II}]=−0.16 V vs SCE) via single electron transfer (SET) and regenerated the active catalyst [QN-H⁺]. Concurrently, formal HAT occurred between III and [Co^{II}], which delivered [Co^{III}-H] and the desired alkylated product 3-H⁺ after rearomatization. The [Co^{III}—H] then reduced the H⁺ and closed the catalytic cycle via releasing H₂.

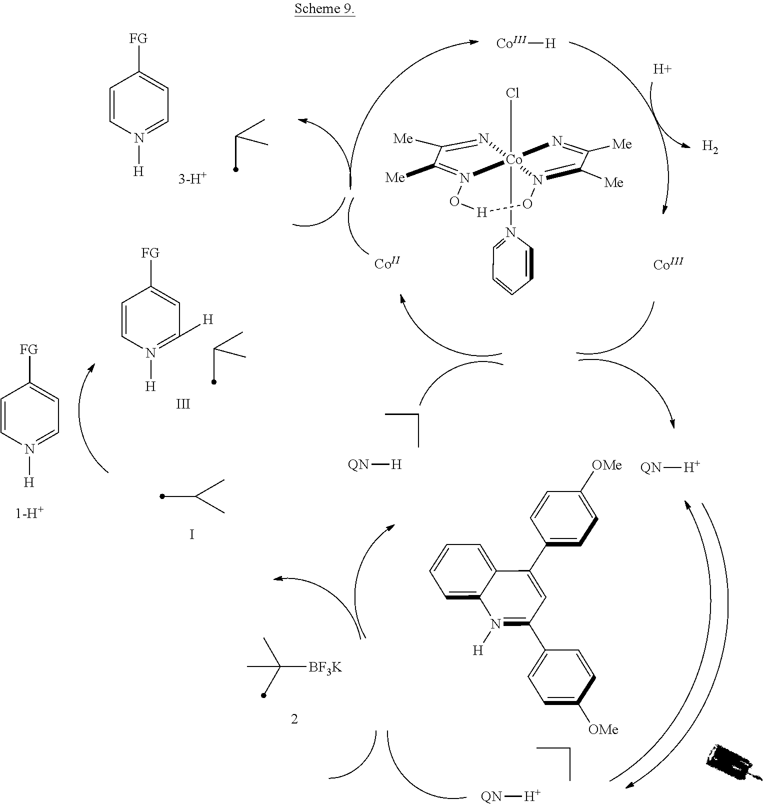

The facile access of different DPQN congeners, which were immobilized on the commercially available amino-modified polystyrene (PS) beads via amide coupling, allowed the convenient preparation of solid-supported organophotocatalysts DPQN^{2,4-di-OR}@PS (Formula VII). To a 10 mL glass vial equipped with a Teflon-coated magnetic stirring bar were added p-hydroxybenzaldehyde (610.6 mg, 5.0 mmol, 1.0 equiv), aniline (465.7 mg, 5.0 mmol, 1.0 equiv), and anhydrous MgSO4 (300.9 mg, 2.5 mmol, 0.50 equiv). Then, CH₂Cl₂ (5.0 mL) was added to the reaction mixture, which was stirred at room temperature. After 2 h, the MgSO₄ was filtered and washed with CH₂Cl₂, and the filtrate was concentrated in a 20 mL glass vial to dryness to afford the crude imine, which was directly used without further purification.

To the crude imine were sequentially added 4-ethynylanisole (1.3 mL, 10 mmol, 2.0 equiv), Fe(OTf)₃ (62.9 mg, 375 μmol, 2.5 mol %), toluene (5.0 mL) and AcOH (428.9 mL, 0.75 mmol, 1.5 equiv). The reaction mixture was gradually heated from 90° C. to 140° C. (Scheme 10). After being stirred at 140° C. for 16 h, the insolubles were filtered with a short celite pad and washed with EtOAc. The filtrate was basified with sat NaHCO₃ (aq) and extracted with EtOAc. The organic layer was dried over an-hydrous MgSO4 and concentrated. The residue was then purified by column chromatography (Hex:EtOAc=10:1 to 5:3) and recrystallized with pentane/Et2O to afford the pure DPQN^{2-OR-4-OMe} (0.41 g, 25%).

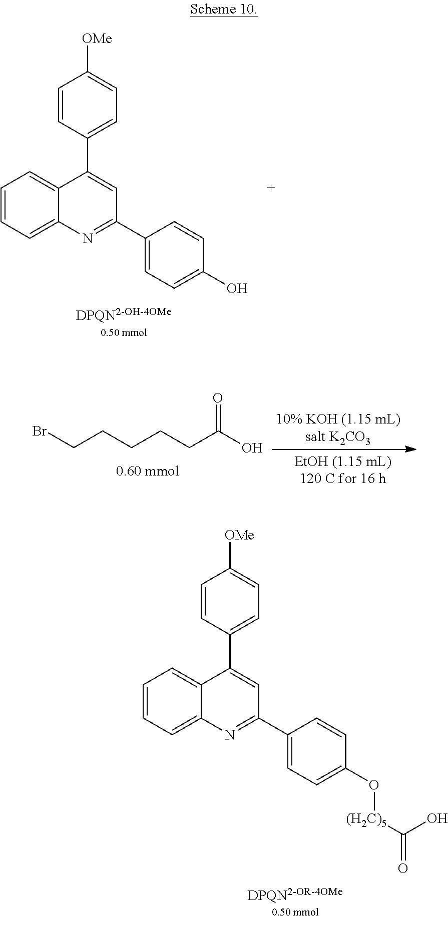

To a 10 mL pyrex microwave tube equipped with a Teflon-coated magnetic stirring bar were added DPQN^{2-OR-4-OMe} (163.7 mg, 0.50 mmol, 1.0 equiv), 6-bromohexanoic acid (84 μL, 0.60 mmol, 1.2 equiv), 10 wt % KOH (1.25 mL), sat K₂CO₃ (0.50 mL), and EtOH (1.5 mL). After being stirred at 120° C. for 16 h, the reaction mixture was acidified with 18 wt % HCl to pH 5.0 to 6.0 and extracted with EtOAc. The organic layer was dried over anhydrous MgSO₄ and concentrated. The residue was then purified by column chromatography (Hex:EtOAc=10:1 to 5:3) and recrystallized with pentane/Et₂O to afford the pure DPQN^{2-OR-4-OMe} (66.3 mg, 30%).

To a 10 mL glass vial equipped with a Teflon-coated magnetic stirring bar were added DPQN^{2-OR-4-OMe} (44.1 mg, 0.10 mmol, 1.0 equiv) and dichloroethane (5.0 mL), which was followed by the dropwise addition of oxalyl chloride (16 μL, 0.20 mmol, 2.0 equiv) and 1 drop dimethylformamide. After being stirred for 6 h, to the reaction mixture were added (aminomethyl)polystyrene (250 mg, Sigma Aldrich, purchase ID:515620) and Et₃N (0.70 mL, 0.50 mmol). After being stirred at 50° C. for 8 h, the reaction was quenched by benzoyl chloride (87 μL, 0.75 mmol, 7.5 equiv) and Et₃N (292 μL, 0.75 mmol, 7.5 equiv), and kept stirring at 50° C. After 2 h, the insoluble beads were washed with MeOH, water, acetone, and CH₂Cl₂. After drying at 60° C. for 3 h, pale yellow DPQN^{2,4-di-OR@PS} beads were obtained (408.1 mg). The filtrate was concentrated and taken for ¹H NMR analysis using CH₂Br₂ as the internal standard, which showed that 69% of the starting DPQN^{2-OR-4-OMe} was recovered.

The increased weight of PS beads after the reaction (250 mg vs 408.1 mg) comes from 1) installation of DPQN^{2-OR-4-OMe}) the benzoyl protecting groups of the amine residues on the surface. We assumed that the non-recovered DPQN^{2-OR-4-OMe} (31%, 0.031 mmol) were all on the PS beads. Therefore, the loading of DPQN^{2-OR-4-OMe} on DPQN2,4-di-OR@PS is 0.031 mmol/408.1 mg=7.6·10-5 mmol/mg.

Robustness tests showed that the DPQN^{2,4-di-OR}@PS in only 0.50 mol % loading could be used for oxidative Minisci alkylation five times after simple filtration (Scheme 11, Table 8). Compound 1a was alkylated with compound 2a (1.5 eq.) with a catalyst system of 0.50 mol % DPQN^{2-OR-4-OMe} @PS and 5.0 mol % of chloro(pyridine)bis(dimethylglyoximato)cobalt (III). The standard conditions of light stimulus (>395 nm), TFA (2.0 equiv) dioxane (0.067 M) at room temperature for 20 h were applied.

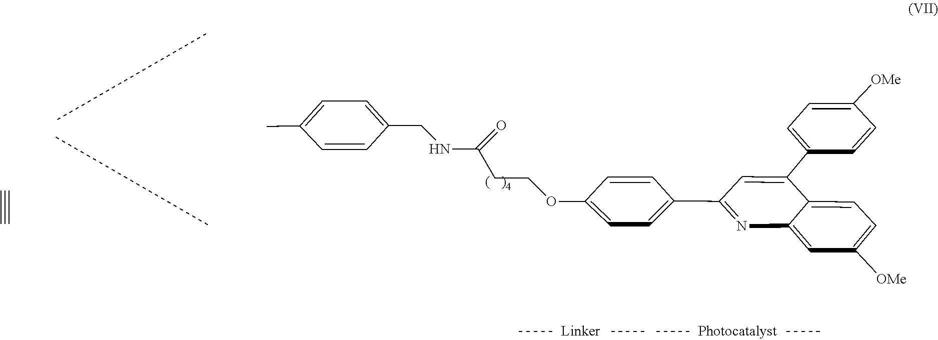

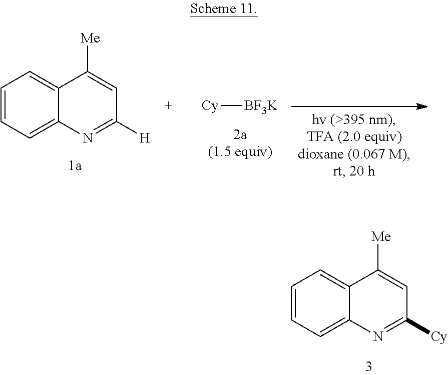

TABLE 8

| Recycle cycle number | NMR yield of compound 3 |
|---|---|
| 1 | 86% |
| 2 | 84% |
| 3 | 77% |
| 4 | 65% |
| 5 | 50% |

The photosynthetic versatility and generality of DPQN^{2,4-di-OMe}-based oxidative coupling platform were further explored by harnessing its oxidatively initiated reactivities with more radical alkylating reagents (Table 9). In terms of radical donors, C₄-alkylated Hantzsch esters showed comparable productivity in heteroaromatic C—H substitution in place of the R-BF₃K (Table 9). Most of the prior established (fluoro)alkylation methods using sulfinate-derived radicals were operated with oxidants. Through the present dual catalytic platform, (fluoro)alkylated products, including tert-butylated lepidine (compound 7), the high-value trifluoromethylated dipeptide (compound 58) and difluoromethylated caffeine (compound 59), were obtained in an H₂-releasing manner. A TfNHNHBoc reagent was exploited to expedite the trifluoromethyl radical, which was captured by 1,3,5-trimethoxybenzene to afford compound 60. Interestingly, when the non-protected Boc-hydrazide was applied directly, the tert-butylated product was obtained. The quinoline/cobalt co-catalyzed system could also accommodate other attractive alkylating reagents, which liberated the desired radicals driven by either restoring aromaticity or extruding CO₂ (Table 9).

Of equal importance, other types of DPQN^{2,4-di-OMe}-catalyzed photoreactions were investigated with Cy-BF₃K and suitable radical acceptors with unsaturated bondings (Tables 9 and 10). Giese-type addition of cyclohexyl radical (Cy⁻) to electron-poor C═C double bond was found amenable, which could trigger a cascade radical addition to the pendent benzene ring and accomplish the tandem alkene dicarbofunctionalization. Accordingly, the synthesis of several fused heterocycles was succeeded under the optimal conditions (compounds 61 to 64, Tables 9 and 10). The cyclisation was catalyzed prior to the Giese-type addition and together formed a cascade reaction. Similarly, alkyne could behave as the SOMOphile, furnishing the polycyclic arene smoothly by the photochemical manifold (compound 65).

TABLE 9
Results for the alkylation with alkylation precursors

| Compound | Synthesized compound (i.e. | | |
|---|---|---|---|
| number | alkylated substrate) | Alkylation precursor | Yield |
| 37 | 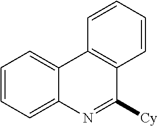 | 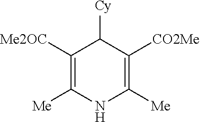 | 86% |
| 7 | 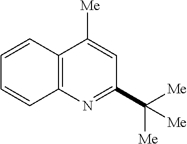 | 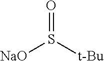 | 57% |
| 58 | 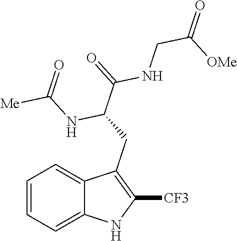 | 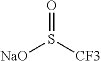 | 45% |
| 59 | 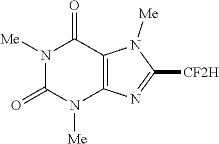 | 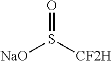 | 42% |
| 60 | 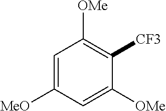 | 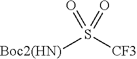 | 60% |
| 7 | 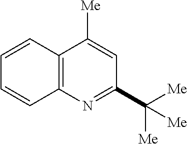 | 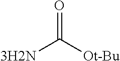 | 44% |
| 7 | 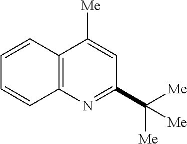 | 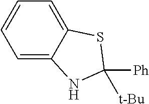 [SMILES: CC(C)(C)C1(c2ccccc2)Nc2ccccc2S1] | 77% |
| 4 | 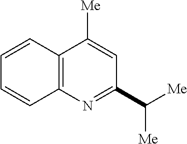 | 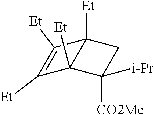 [SMILES: CCC1=C(CC)C2(CC)C1(CC)CC2(C(=O)OC)C(C)C] | 66% |
| 3 | 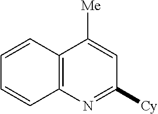 | 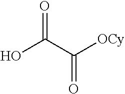 | 39% |
| 4 | 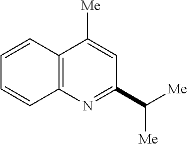 | 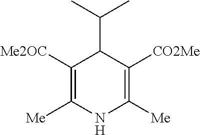 | 84% |
| 5 | 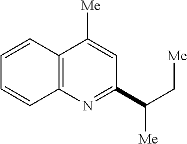 | 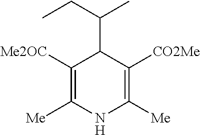 | 90% |

TABLE 10
Results for substrate internal cyclisation

| Compound | Synthesized compound (i.e. | | |
|---|---|---|---|
| number | alkylated substrate) | Substrate for cyclisation/alkylation | Yield |
| 61 | 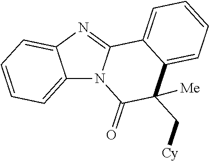 | 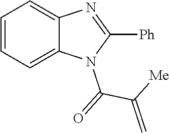 [SMILES: C=C(C)C(=O)n1c(-c2ccccc2)nc2ccccc21] | 68% |
| 62 | 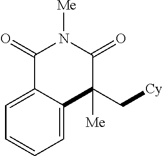 | 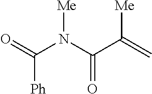 | 59% |
| 63 | 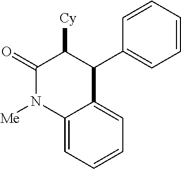 | 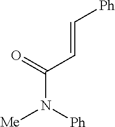 [SMILES: CN(C(=O)/C=C/c1ccccc1)c1ccccc1] | 49% |
| 64 | 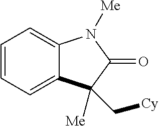 | 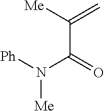 | 54% |
| 65 | 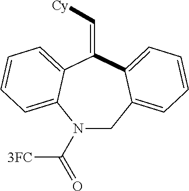 | 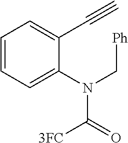 | 45% |

It is noted that in the present example, stoichiometric chemical oxidants were unnecessary for balancing the redox status in all cases of oxidative couplings above. Collectively, the present example illustrated the tremendous synthetic capabilities of compounds of formula I, and particularly DPQN^{2,4-di-OMe}. DPQN^{2,4-di-OMe} is a photoredox catalyst based on diarylquinoline, which was enabled oxidatively initiated alkylation chemistry. Furthermore, DPQN^{2,4-di-OMe} was successfully synthesized via a three-component coupling of the corresponding aldehyde, alkyne and amine (scheme 2). The present example has established a visible light-mediated dehydrogenative Minisci alkylation between heteroarene and a numerous carbon radical precursors in a catalytic combination of formula I and cobaloxime. The present catalyst system of formula I and cobaloxime empowers a set of photoredox reactions for C—C bond formation without chemical oxidants, wherein, the carbon radicals were intercepted by other radical acceptors for different synthetic purposes. The computed S0-T1 gap of DPQN2,4-di-OMe estimated its triplet energy (ET) to be 52.2 kcal/mol, which was similar to its structurally related acridinium photocatalysts, indicating that it serves as a prominent photosensitizer for triplet energy transfer (EnT).

In the following experiments, photoreactions with DPQN^{2,4-di-OMe} in the absence of a co-catalyst (i.e. no cobalt organocatalyst) were performed. Scheme 12 shows the alkylation reaction between the substrate α-trifluoromethylstyrene and Cy-BF₃K. To a 10 mL pyrex microwave tube equipped with a Teflon-coated magnetic stirring bar were added alkyl α-trifluoromethylstyrene (0.10 mmol, 1.0 equiv), potassium cyclohexyltrifluoroborate (28.5 mg, 0.15 mmol, 1.5 equiv), and DPQN^{2,4-di-OMe} (1.7 mg, 5.0 mmol, 5.0 mol %). The tube was sealed with a rubber septum, evacuated and backfilled with argon three times before dioxane (1.5 mL) was injected into the reaction tube. Then, to the mixture was added TFA (7.7 mL, 0.10 mmol, 1.0 equiv) in the glovebox. After that, the reaction tube was sealed with an aluminum cap with a septum, which was taken out from the glovebox and stirred at ˜37° C. under a 300 W Xe lamp irradiation with a 395 nm filter. After 20 h, the reaction mixture was basified with saturated NaHCO₃ aqueous solution, extracted with EtOAc, filtered through a short pad of MgSO₄, and concentrated to obtain the crude product. The product was isolated by preparative thin-layer chromatography.

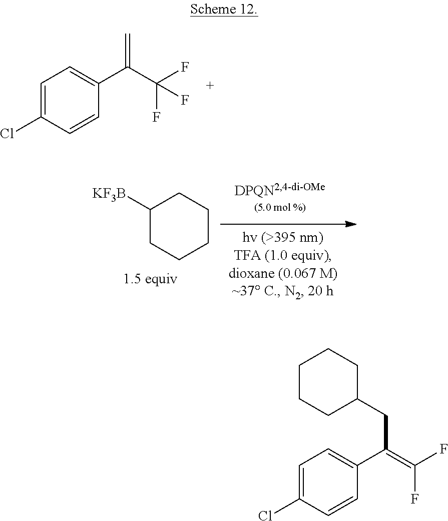

Scheme 13 shows the procedure for the coupling of benziodoxolones and cyclohexyltrifluoroborate. To a 10 mL pyrex microwave tube equipped with a Teflon-coated magnetic stirring bar were added alkenyl/alkynyl alkyl benziodoxolones (0.10 mmol, 1.0 equiv), potassium cyclohexyltrifluoroborate (28.5 mg, 0.15 mmol, 1.5 equiv), and DPQN^{2,4-di-OMe} (1.7 mg, 5.0 mmol, 5.0 mol %). The tube was sealed with a rubber septum, evacuated and backfilled with argon three times before dioxane (1.5 mL) was injected into the reaction tube. Then, to the mixture was added TFA (7.7 mL, 0.10 mmol, 1.0 equiv) in the glovebox. After that, the reaction tube was sealed with an aluminum cap with a septum, which was taken out from the glovebox and stirred at ˜37° C. under a 300 W Xe lamp irradiation with a 395 nm filter. After 20 h, the reaction mixture was basified with saturated NaHCO₃ aqueous solution, extracted with EtOAc, filtered through a short pad of MgSO₄, and concentrated to obtain the crude product. The product was isolated by preparative thin-layer chromatography.

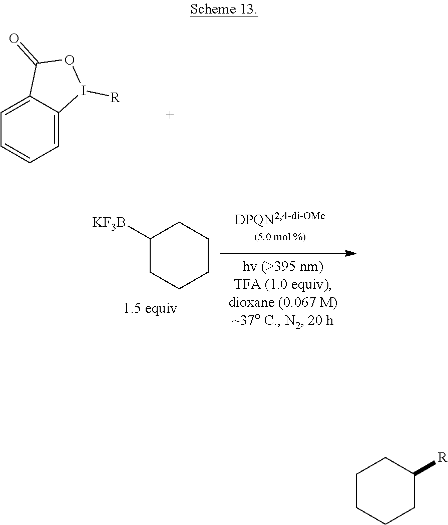

Scheme 14 shows the procedure for BocN=NBoc and Cy-BF₃K. To a 10 mL pyrex microwave tube equipped with a Teflon-coated magnetic stirring bar were added di-tert-butyl azodicarboxylate (23.0 mg, 0.10 mmol, 1.0 equiv), potassium cyclohexyltrifluoroborate (28.5 mg, 0.15 mmol, 1.5 equiv), and DPQN^{2,4-di-OMe} (1.7 mg, 5.0 mmol, 5.0 mol %). The tube was sealed with a rubber septum, evacuated and backfilled with argon three times before dioxane (1.5 mL) was injected into the reaction tube. Then, to the mixture was added TFA (7.7 mL, 0.10 mmol, 1.0 equiv) in the glovebox. After that, the reaction tube was sealed with an aluminum cap with a septum, which was taken out from the glovebox and stirred at ˜37° C. under a 300 W Xe lamp irradiation with a 395 nm filter. After 20 h, The reaction mixture was basified with saturated NaHCO₃ aqueous solution, extracted with EtOAc, filtered through a short pad of MgSO₄, and concentrated to obtain the crude product. The product was isolated by column chromatography.

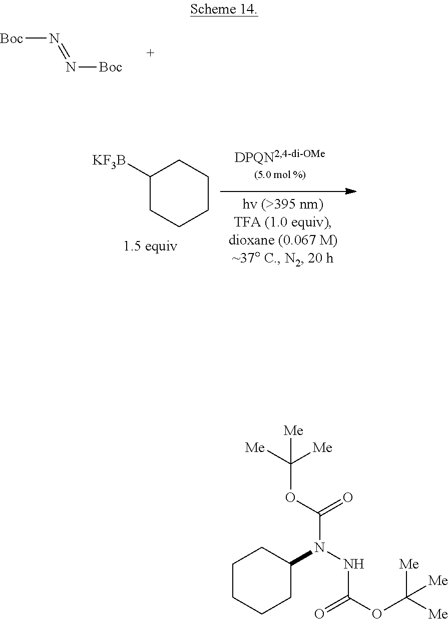

Scheme 15 shows the procedure for the coupling of alkyl sulfonothioates/sulfonoselenoate and cyclohexyltrifluoroborate. To a 10 mL pyrex microwave tube equipped with a Teflon-coated magnetic stirring bar were added alkyl sulfonothioate/sulfonoselenoate (0.10 mmol, 1.0 equiv), potassium cyclohexyltrifluoroborate (28.5 mg, 0.15 mmol, 1.5 equiv), DPQN^{2,4-di-OMe} (1.7 mg, 5.0 mmol, 5.0 mol %) and [Co(dmgH)₂(py)]Cl (2.0 mg, 5.0 mmol, 5.0 mol %). The tube was sealed with a rubber septum, evacuated and backfilled with argon three times before dioxane (1.5 mL) was injected into the reaction tube. Then, to the mixture was added TFA (7.7 mL, 0.10 mmol, 1.0 equiv) in the glovebox. After that, the reaction tube was sealed with an aluminum cap with a septum, which was taken out from the glovebox and stirred at ˜37° C. under a 300 W Xe lamp irradiation with a 395 nm filter. After 20 h, The reaction mixture was basified with saturated NaHCO₃ aqueous solution, extracted with EtOAc, filtered through a short pad of MgSO₄, and concentrated to obtain the crude product. The product was isolated by preparative thin-layer chromatography or column chromatography.

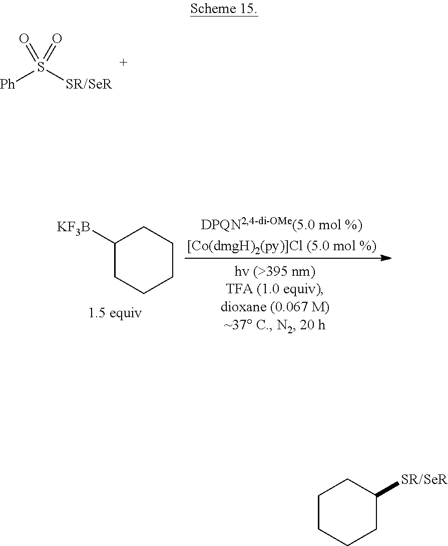

Table 11 shows additional cyclohexyl addition performed without co-catalyst but with DPQN^{2,4-di-OMe} (1.7 mg, 5.0 mmol, 5.0 mol %) and [Co(dmgH)₂(py)]Cl (2.0 mg, 5.0 mmol, 5.0 mol %). The cyclohexyl additions summarized in Table 11 used Cy-BF₃K as the alkylation precursor.

TABLE 11
Cyclohexyl addition without a co-catalyst

| Substrate with Cy addition | Substrate | Yield |
|---|---|---|
| 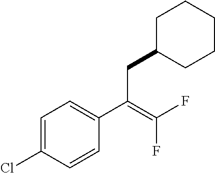 | 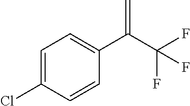 | 61% |
| 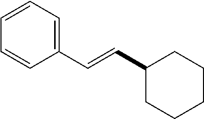 | 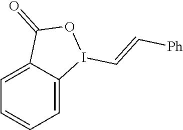 [SMILES: O=C1OI(/C=C/c2ccccc2)c2ccccc21] | 38% |
| 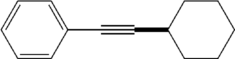 | 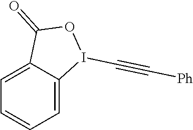 [SMILES: O=C1OI(C#Cc2ccccc2)c2ccccc21] | 47% |
| 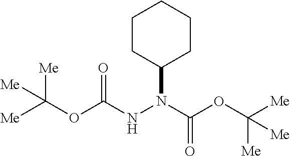 | 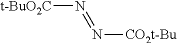 | 51% |
| 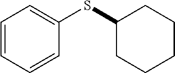 | 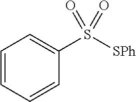 | 75% |
| 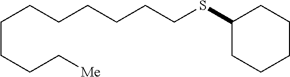 | 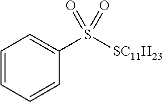 | 80% |
| 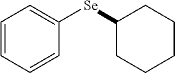 | 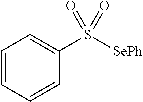 | 84% |

Scheme 16 shows the procedure for a trifluoromethylation. The organophotoredox catalyst used was a phenyl pyridine quinolone with two OMe groups (PPQN^{2,4-di-OMe}) as shown in scheme 17 which shows the equilibrium between the organophotoredox catalyst and the nickel complex that can form (metallophotoredox catalyst). To a 10 mL pyrex microwave tube equipped with a Teflon-coated magnetic stirring bar were added NiCl₂·glyme (1.1 mg, 5.0 μmol, 5.0 mol %) and PPQN^{2,4-di-OMe}(1.7 mg, 5.0 μmol, 5.0 mol %) in DCM (0.50 mL), which was stirred for 30 minutes to pre-form the complex. The volatiles were then removed under vacuum. Afterward, to a reaction tube were added 1,3,5-trimethoxybenzene (16.8 mg, 0.10 mmol, 1.0 equiv), NaSO₂CF₃ (46.8 mg, 0.30 mmol, 3.0 equiv) and K₂S₂O₈(27.0 mg, 0.10 mmol, 1.0 equiv). The tube was sealed with a rubber septum, evacuated and backfilled with argon three times before DMSO (1.0 mL) was injected into the reaction tube. Then, the tube was moved in the glovebox, where it was sealed with an aluminum cap with a septum. After that, it was taken out from the glovebox, stirred at ˜37° C. and irradiated by a Kessil lamp (λₘₐₓ=390 nm, 50 W). After 20 h, the reaction mixture was filtered through a short pad of silica gel, and concentrated to obtain the crude product. The product was isolated by preparative thin-layer chromatography or column chromatography.

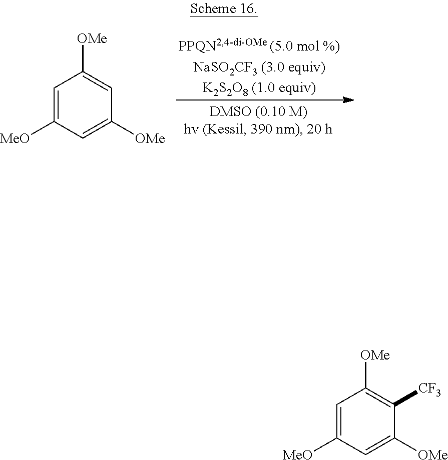

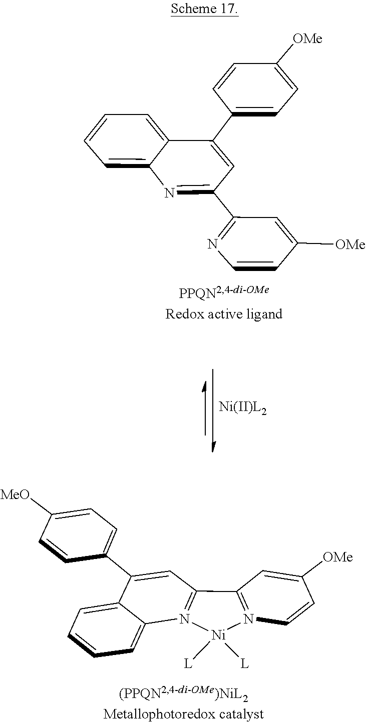

Scheme 18 shows a pinacol coupling with PPQN^{2,4-di-OMe}. To a 10 mL pyrex microwave tube equipped with a Teflon-coated magnetic stirring bar were added NiCl₂-glyme (1.1 mg, 5.0 μmol, 5.0 mol %) and PPQN^{2,4-di-OMe}(1.7 mg, 5.0 μmol, 5.0 mol %) in DCM (0.50 mL), which was stirred for 30 minutes to pre-form the complex. The volatiles were then removed under vacuum. Afterward, to a reaction tube were added benzophenone (36.4 mg, 0.20 mmol, 2.0 equiv) and (n-Bu)₃N (55.5 mg, 0.30 mmol, 3.0 equiv). The tube was sealed with a rubber septum, evacuated and backfilled with argon three times before N,N-dimethylformamide (DMF) (1.0 mL) was injected into the reaction tube. Then, the tube was moved in the glovebox, where it was sealed with an aluminum cap with a septum. After that, it was taken out from the glovebox, stirred at ˜37° C. and irradiated by a Kessil lamp (λₘₐₓ=390 nm, 50 W). After 20 h, the reaction mixture was quenched by water, extracted by EtOAc. The collected organic layers were combined and concentrated to obtain the crude product. The product was isolated by preparative thin-layer chromatography or column chromatography.

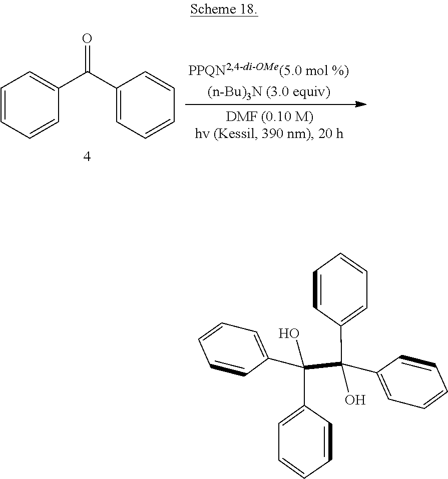

### Example 2

A photoactive ligand is shown herein to complex with a series of transition metals and serve as a “two-in-one” metallophotoredox catalyst. This bifunctional system is compatible with a diverse pool of nucleophilic and electrophilic coupling partners and highly enabling in visible-light-driven C—C and C—X bond formations. Upon complexation, the metal-ligand assembly was shown to switch on its photoexcitation mode, exhibiting potent photochemical properties under light irradiation while preserving its cross-coupling capability. Such a merger brings additional benefits of improving the reaction efficiency since the metal centers neighbor the nascent radicals, thus, better managing the interlocked cycles mediated by light and metal, respectively. Moreover, in transition Metal™ catalysis, the light facilitates some elementary yet orthogonal organometallic steps simultaneously (e.g., transmetallation, oxidative addition, and reductive elimination) via open-shell intermediacy. The present example shows the design of such versatile ligands, the metal complex of which can confine the dual metallophotoredox reactivities (e.g., electron, energy, and radical transfers) into a singular catalytic entity. Under the monocatalytic conditions tested herein, a diverse reactivity profile was accessed simply by changing the metal precatalysts and coupling partners, thereby improving the synthetic proficiencies for reactions of high interest.

Nickel/bipyridine, due to its versatility and availability, enjoys a privileged role as the TM catalyst. However, the fact that most of these complexes feature strong absorptivity only in the ultraviolet region, which is governed by the ligand-oriented p-p* transition, dictated the presence of an external photocatalyst (PC) for visible light absorption. Besides, compared with those coordinatively saturated PCs, the short-lived excited state of substitution-labile nickel complexes and their slow photokinetics of intersystem crossing (ISC) compromised their photosynthetic application in their own right. Likewise, the studies on the photocatalysis of other non-noble metal coordination compounds lagged behind.

In light of these limitations, it was hypothesized that engineering the bipyridine scaffold could provide an alternative avenue to enlighten the nickel photochemistry, therefore, enabling some previously elusive transformations in the classic regime of nickel/bipyridine catalysis. Considering Example 1 which showed that diarylquinolines could behave as efficient PCs upon Brønsted acid activation, it was investigated whether the repurposed diarylquinolinium with an embedded bipyridine motif could impart similar photochemical reactivities when chelating with Lewis acidic TMs. The chemical possibilities of metalated diarylquinolinium were expanded herein, and were geared with the capacity of fragment couplings owing to the vacant coordination sites.

The synthesis of several 4-phenyl-2-(pyridin-2-yl)quinolines (PPQNs) was performed. Different members in this ligand set were prepared from two readily available and low-cost ketone building blocks (see the two compounds below) via Friedlsnder condensation.

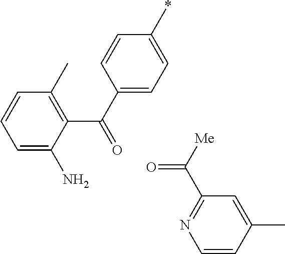

In contrast to other synthetic routes for bipyridine modifications, noble metals are absent in the present case, therefore, circumventing issues caused by metal residues and simplifying their purification.

Solvents used in the present Example were stored over 4 Å molecular sieves (beads, 8-12 mesh) and degassed by purging with argon for 30 min. The 4 Å molecular sieves were purchased from Sigma-Aldrich™ and activated in the oven for 12 h at 380° C. before use. Reagents were purchased from Sigma-Aldrich™, Combi-Blocks™, TCI America™, Oakwood™, and Fisher Scientific™ and used without further purification unless otherwise specified.

Nuclear magnetic resonance (NMR) spectra, including ¹H NMR, ¹³C NMR, and ¹⁹F NMR, were recorded on Bruker™ 500 MHz spectrometers, using the deuterium lock signal to reference the spectra. The solvent residual peaks, e.g., chloroform (CDCl₃: δ 7.28 ppm and δ 77.02 ppm), were used as references. Data was reported as follows: multiplicity (s=singlet, d=doublet, t=triplet, q=quartet, quint=quintet, m=multiplet, dd=doublet of doublet, etc), coupling constant (J/Hz) and integration. All NMR spectra were recorded at room temperature.

Gas chromatography-mass spectroscopy (GC-MS) was obtained from the Agilent™ gas chromatography-mass spectroscopy system with helium (He) as the carrier gas. Highresolution mass spectrometry (HRMS) was conducted by using atmospheric pressure chemical ionisation (APCI) or electro-spraying ionisation (ESI) and was performed by McGill University on a Thermo-Scientific™ Exactive Orbitrap™. Protonated/deprotonated molecular ions (M±H)⁺ or sodium adducts (M+Na)⁺ were used for empirical formula confirmation. Electrochemical experiments were performed with HEKA™ PG 340 potentiostat with Ag/AgCl as the reference electrode. The working electrode was made of glassy carbon, and a Pt wire was used as the counter electrode to complete the electrochemical setup. A scan rate of 100 mV/s was used for all experiments. All the potentials were noted with respect to the Ag/AgCl electrode unless otherwise specified. The reduction potential referenced to the standard calomel electrode (SCE) was calculated by subtracting 0.039 V from the E(Ag/AgCl). It followed that E(SCE)=E(Ag/AgCl)−0.039 V.

Reactions were stirred magnetically and conducted in 10 mL pyrex sealed tubes under an inert atmosphere unless otherwise specified. Experiments under light irradiation were performed using a 390 nm PR160L Kessil™ lamp equipped with a cooling fan for efficient temperature maintenance. Column chromatography was performed with E. Merck™ silica gel 60 (230-400 mesh).

Dimethoxylated PPQN (PPQN^{2,4-di-OMe}) was first prepared on a gram scale (scheme 19), and its solid-state structure was confirmed by X-ray crystallography (FIG. 6 and Table 12). The synthesis of Ni²⁺/PPQN^{2,4-di-OMe} ligand was performed as follows. To a 25 mL glass tube equipped with a Teflon-coated magnetic stirring bar were added (2-aminophenyl)(4-methoxyphenyl)methanone (1.0 g, 4.4 mmol, 1.0 equiv), 1-(4-methoxy-pyridin-2-yl)-ethanone (0.87 g, 5.8 mmol, 1.3 equiv), AcOH (8.8 mL), and H₂SO₄ (5 drops). The tube was filled with argon and sealed by an aluminium cap with a septum, then stirred at 140° C. for 16 h. Upon completion, the reaction mixture was carefully basified with 10 M NaOH_{(aq)} at 0° C., extracted with EtOAc, dried over anhydrous MgSO₄ and concentrated. The residue was then purified by column chromatography (Hex:EtOAc=20:1 to 1:1) and recrystallised with hexane/EtOAc to afford the pure PPQN^{2,4-di-OMe} (0.92 g, yield 61%).

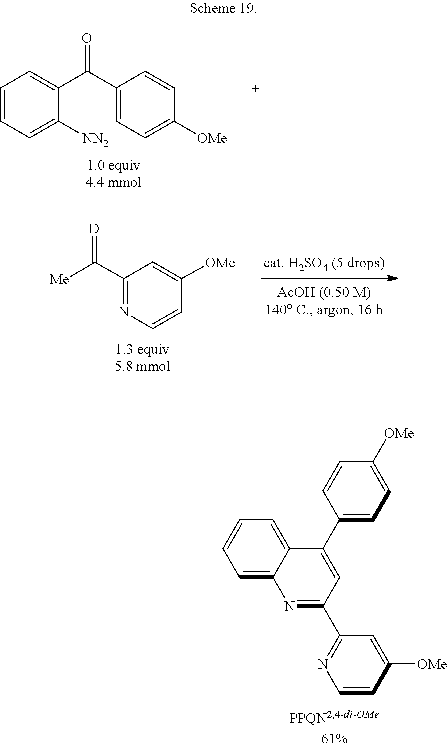

TABLE 12
Crystal data and structure refinement for PPQN^{2,4-di-OMe} by X-ray crystallography

| Empirical formula | C₂₂H₁₈N₂O₂ |
|---|---|
| Formula weight | 342.38 |
| Temperature | 298(2) |
| Crystal system | triclinic |
| Space group | P-1 |
| a/Å | 6.38850(10) |
| b/Å | 10.9059(2) |
| c/Å | 13.2352(3) |
| α/° | 106.3910(10) |
| β/° | 94.7820(10) |
| γ/° | 98.7600(10) |
| Volume/Å³ | 866.53(3) |
| Z | 2 |
| ρ_{calc}g/cm³ | 1.312 |
| μ/mm⁻¹ | 0.679 |
| F(000) | 360.0 |
| Crystal size/mm³ | 0.221 × 0.088 × 0.048 |
| Radiation | CuKα (λ = 1.54178) |
| 2Θ range for data collection/° | 7.024 to 144.55 |
| Index ranges | −7 ≤ h ≤ 6, −13 ≤ k ≤ 13, −16 ≤ l ≤ 16 |
| Reflections collected | 25833 |
| Independent reflections | 3414 [Rᵢₙₜ = 0.0615, R_{sigma} = 0.0320] |
| Data/restraints/parameters | 3414/0/238 |
| Goodness-of-fit on F2 | 1.052 |
| Final R indexes [I >= 2σ (I)] | R₁ = 0.0512, wR₂ = 0.1330 |
| Final R indexes [all data] | R₁ = 0.0856, wR₂ = 0.1774 |
| Largest diff. peak/hole/e Å⁻³ | 0.25/−0.20 |

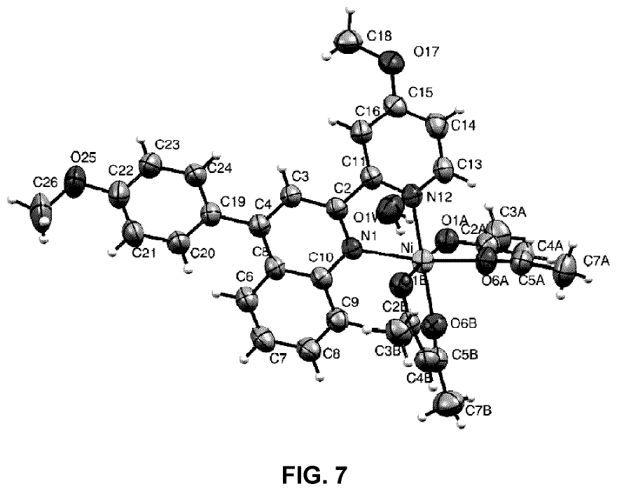

Unless otherwise specified, Ni²⁺/PPQN^{2,4-di-OMe} was made by pre-stirring equimolar NiCl₂·1,2-dimethoxyethane (DME) and PPQN^{2,4-di-OMe}, and a 390 nm Kessil™ lamp was used as light source. Ni²⁺/PPQN^{2,4-di-OMe} was confirmed by X-ray crystallography (FIG. 7 and Table 13).

TABLE 13
Crystal data and structure refinement for Ni2+/PPQN2,4-
di-OMe by X-ray crystallography

| Empirical formula | C₃₂H₃₄N₂NiO₇ |
|---|---|
| Formula weight | 617.32 |
| Temperature | 298(2) K |
| Crystal system | monoclinic |
| Space group | P2₁/n |
| a/Å | 16.6157(3) |
| b/Å | 7.83850(10) |
| c/Å | 23.5227(4) |
| α/° | 90 |
| β/° | 104.3440(10) |
| γ/° | 90 |
| Volume/Å³ | 2968.14(8) |
| Z | 4 |
| ρ_{calc}g/cm³ | 1.381 |
| μ/mm⁻¹ | 1.366 |
| F(000) | 1296.0 |
| Crystal size/mm³ | 0.171 × 0.049 × 0.045 |
| Radiation | CuKα (λ = 1.54178) |
| 2Θ range for data collection/° | 5.884 to 145.002 |
| Index ranges | −20 ≤ h ≤ 20, −9 ≤ k ≤ 7, −29 ≤ l ≤ 29 |
| Reflections collected | 56727 |
| Independent reflections | 5882 [Rint = 0.0960, Rsigma = 0.0379] |
| Data/restraints/parameters | 5882/0/385 |
| Goodness-of-fit on F2 | 1.045 |
| Final R indexes [I >= 2σ (I)] | R₁ = 0.0474, wR₂ = 0.1247 |
| Final R indexes [all data] | R₁ = 0.0710, wR₂ = 0.1482 |
| Largest diff. peak/hole/e Å⁻³ | 0.40/−0.33 |

Three reactions were tested (schemes 20-22 where DMO=dimethylsulfoxide and DMF=N,N-dimethylformamide) and yields were determined by ¹H NMR using dibromomethane as internal standard. For each of schemes 20-22 tris(2,2′-bipyridyl)ruthenium (II) (Ru(bpy)₃²⁺) was used as a control photocatalyst and the yields are shown in Table 14.

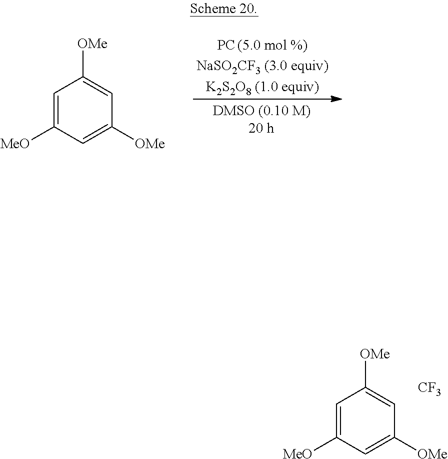

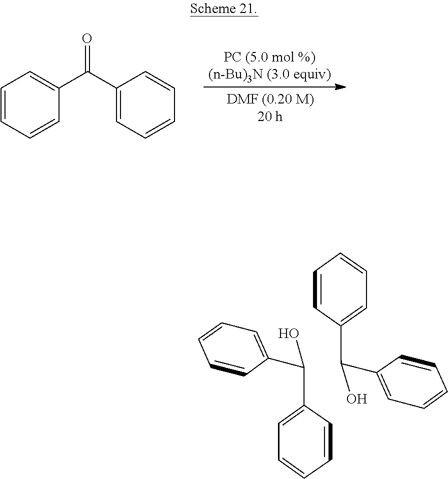

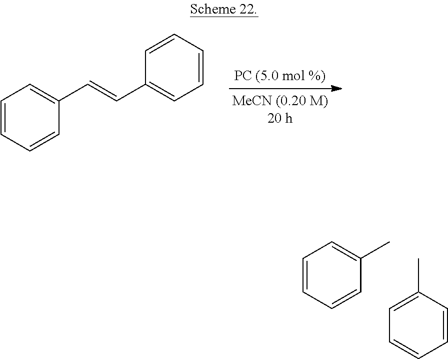

TABLE 14
Yield for schemes 20-22

| Catalyst | Ni²⁺/PPQN^{2,4-di-OMe} | Ru(bpy)₃²⁺ |
|---|---|---|
| Scheme 20 | 50% | 64% |
| Scheme 21 | 50% | 87% |
| Scheme 22 | 67% | 69% |

The Ni²⁺/PPQN^{2,4-di-OMe} was able to furnish the desired products in all cases and with a yield that was comparable with the regularly used Ru(bpy)₃²⁺ PC. The success in verifying the competence of Ni²⁺/PPQN^{2,4-di-OMe} in photocatalysis established the concept of a “two-in-one” metallophotoredox cross-couplings. Mechanistically, Ni²⁺/PPQN^{2,4-di-OMe} could mimic conventional metallophotocatalytic systems with separated roles where part of the Ni²⁺/PPQN^{2,4-di-OMe} mediates the transition Metal™ catalytic cycles, and the rest sustains the photochemical reactions via SET or EnT. Alternatively, the role-unification scenario in which one single Ni²⁺/PPQN^{2,4-di-OMe} controls all metallophotoredox cross-coupling steps via direct excitation could also be plausible.

The SM cross-coupling between iodobenzene and potassiumbenzyltrifluoroborate was tested. The cross-coupling reaction proceeded smoothly with 5.0 mol % Ni²⁺/PPQN^{2,4-di-OMe} under Kessil™ 390 nm light-emitting diodes (LEDs) irradiation, resulting in a 65% yield of diphenylmethane (scheme 23).

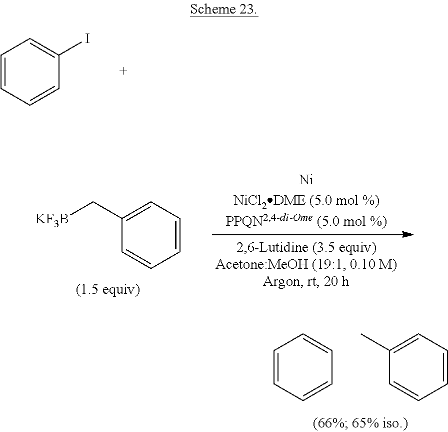

The reaction of Scheme 23 was repeated with 1.0 mol % of [Ir] which is an abbreviation for [4,4′-bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate, without light, without NiCl₂·DME, without ligand, without base or under air instead of argon. The reaction of Scheme 23 was also repeated with different catalysts instead of PPQN^{2,4-di-OMe}, namely the compounds listed below. The resulting yields are presented in Table 15.

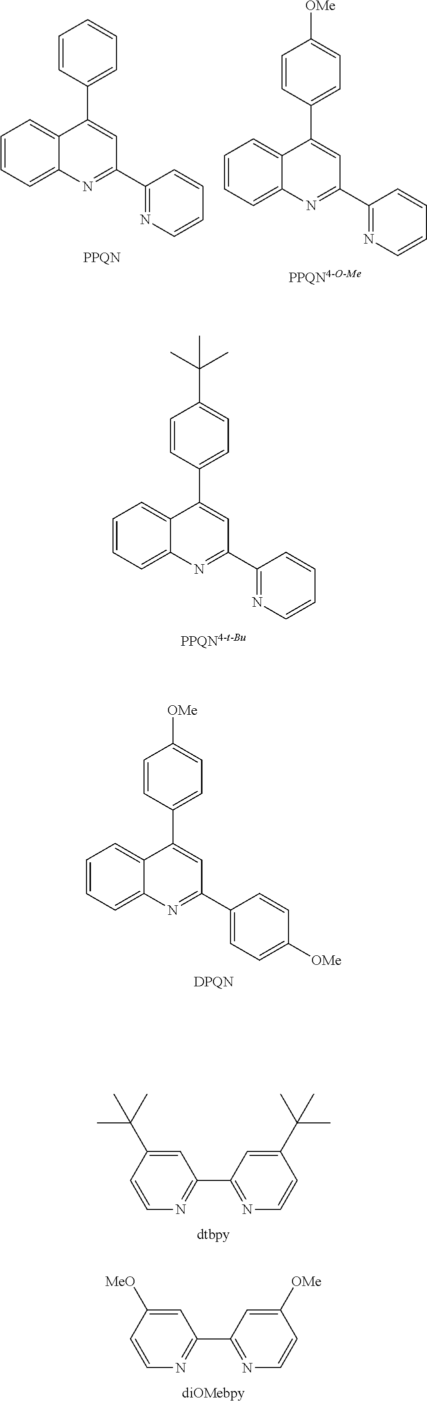

TABLE 15
Yields for Scheme 23 and other conditions tested

| Condition | Yield |
|---|---|
| Scheme 23 | 66% |
| Scheme 23 with 1.0 mol % of [Ir] | 21% |
| Scheme 23 without light | 0% |
| Scheme 23 without NiCl₂•DME | 0% |
| Scheme 23 without ligand | 0% |
| Scheme 23 without base | 8% |
| Scheme 23 under air instead of argon | 0% |
| Scheme 23 with PPQN instead of PPQN^{2,4-di-OMe} | 32% |
| Scheme 23 with PPQN^{4-OMe} instead of PPQN^{2,4-di-OMe} | 44% |
| Scheme 23 with PPQN^{4-t-Bu} instead of PPQN^{2,4-di-OMe} | 20% |
| Scheme 23 with DPQN instead of PPQN^{2,4-di-OMe} | 0% |
| Scheme 23 with dtbpy instead of PPQN^{2,4-di-OMe} | 0% |
| Scheme 23 with diOMebpy instead of PPQN^{2,4-di-OMe} | trace |

Changing the ligands impacted the reaction outcome considerably as the nonsubstituted PPQN, its monomethoxy (PPQN^{4-OMe}), and mono-tert-butyl (PPQN^{4-t-Bu}) variants gave dramatically lower yields. Interestingly, the monodentate DPQN, an organophotoredox catalyst, was ineffective in this coupling reaction, which indicated the importance of bidentate chelation. Commercially available bipyridines (dtbpy and diOMebpy) did not provide the desired reactivities. The addition of extra PC, [Ir(dFCF₃ppy)₂(bpy)]PF₆ decreased the efficiency of SM coupling in our case, presumably owing to the competing light absorption with Ni²⁺/PPQN^{2,4-di-OMe}. Control experiments indicated that light, metal, ligand, base, and inert atmosphere were all indispensable to realize this transformation efficiently.

Based on the determined optimal reaction conditions, substrate scope studies with an array of aryl halides and RBF₃K under Ni²⁺/PPQN^{2,4-di-OMe} metallophotocatalysis were initiated. To begin, the performance of aromatic halides bearing different substitution patterns, including iodides, bromides, and chlorides, was assessed. First, the catalyst was synthesized by pre-stirring PPQN^{2,4-di-OMe} (3.4 mg, 10 μmol, 10 mol %) and NiCl₂·DME (2.2 mg, 10 μmol, 10 mol %) in DMSO (0.50 mL) in a 10 mL pyrex microwave tube for 30 min (scheme 24, oxidative photocatalysis). 1,3,5-Trimethoxybenzene (16.8 mg, 0.10 mmol, 1.0 equiv), NaSO₂CF₃ (46.8 mg, 0.30 mmol, 3.0 equiv) and K₂S₂O₈ (27.0 mg, 0.10 mmol, 1.0 equiv) were then added. The tube was then sealed with a rubber septum, degassed by three freeze-pump-thaw cycles, back-filled with argon, and stirred at room temperature under the 53 W 390 nm LED irradiation. After 20 h, to the reaction mixture was added brine, which was extracted with EtOAc, filtered through a short pad of MgSO₄, and concentrated to afford the crude product. The ¹H NMR yield was determined using CH₂Br₂ as the internal standard to be 50% and 0% in a negative control condition without irradiation.

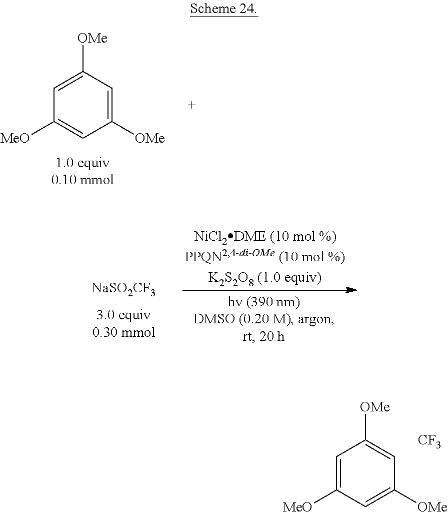

As explained above, the catalyst was synthesized by pre-stirring PPQN^{2,4-di-OMe} (3.4 mg, 10 μmol, 10 mol %) and NiCl₂·DME (2.2 mg, 10 μmol, 10 mol %) in DMF (1.0 mL) in a 10 mL pyrex microwave tube for 30 min. Benzophenone (36.4 mg, 0.20 mmol, 1.0 equiv) and tributylamine (143 μL, 111.0 mg, 0.60 mmol, 3.0 equiv) were then added (scheme 25, reductive photocatalysis). The tube was then sealed with a rubber septum, degassed by three freeze-pump-thaw cycles, back-filled with argon, and stirred at room temperature under the 53 W 390 nm LED irradiation. After 20 h, to the reaction mixture was added brine, which was extracted with EtOAc, filtered through a short pad of MgSO₄, and concentrated to afford the crude product. The ¹H NMR yield was determined using CH²Br² as the internal standard to be 50% and the negative control without irradiation had a 0% yield.

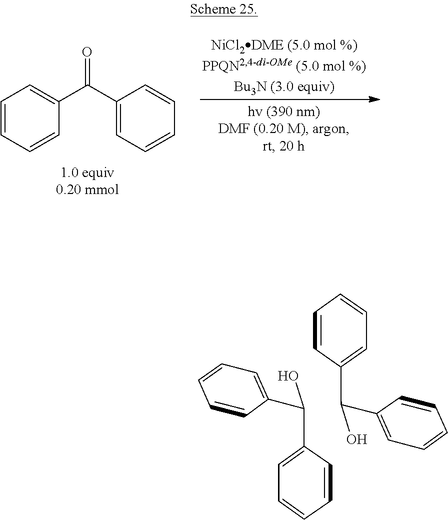

In another experiment, the catalyst was synthesized by pre-stirring PPQN^{2,4-di-OMe} (1.7 mg, 5.0 μmol, 5.0 mol %) and NiCl₂·DME (1.1 mg, 5.0 μmol, 5.0 mol %) in CH₂Cl₂ (1.0 mL) in a 10 mL pyrex microwave tube for 30 min as explained above. (E)-Stilbene (18.0 mg, 0.10 mmol, 1.0 equiv) and MeCN (1.0 mL) were then added (scheme 26, energy-transfer photocatalysis). The tube was then sealed with a rubber septum, degassed by three freeze-pump-thaw cycles, back-filled with argon, and stirred at room temperature under the 53 W 390 nm LED irradiation. After 20 h, the reaction mixture was passed through a short pad of silica gel and concentrated to afford the crude product. The ¹H NMR yield was determined using CH²Br² as the internal standard to be 67% and the negative control without irradiation had a 0% yield.

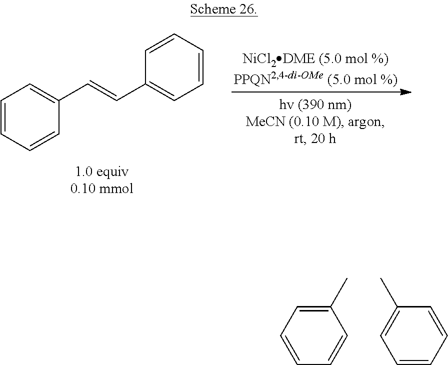

Control experiments for the above three reactions (Schemes 24-26) were performed by replacing the NiCl₂ with a Brønsted acid (trifluoroacetic acid, TFA, used in this case), schemes 27-30. Under otherwise standard conditions, aromatic trifluoromethylation and olefin E/Z isomerisation proceeded smoothly, which was consistent with the previous experiments showing that the diarylquinolinium enables oxidative photoredox catalysis and energy-transfer catalysis.

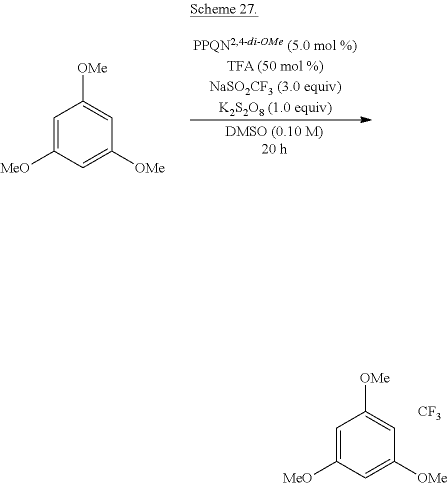

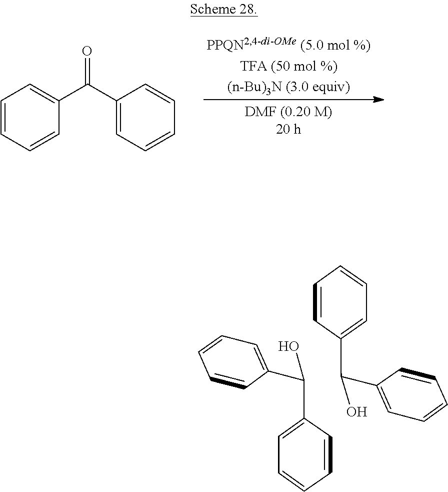

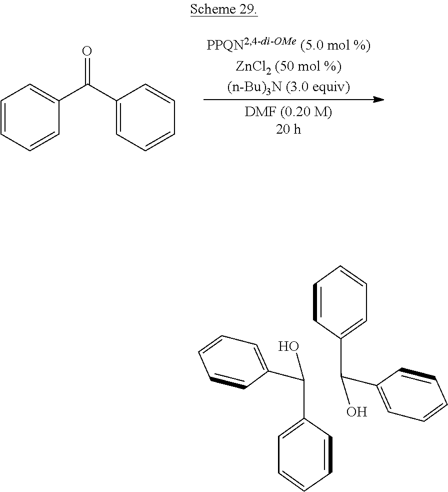

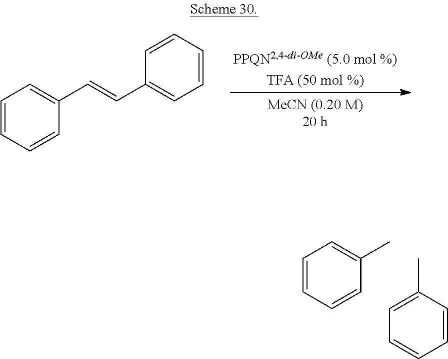

However, reductive pinacol coupling was unsuccessful with protonated or Zn²⁺-activated PPQN^{2,4-di-OMe}. Since organic base n-Bu₃N was used as the terminal reductant in this transformation, its competition with PPQN^{2,4-di-OMe}for strong Brønsted acids or Lewis acids might deactivate the photoredox system and also undermine its single-electron transfer. Therefore, the moderately Lewis acidic Ni²⁺ was uniquely enabling in this reductive coupling. As such, transition metals might not be needed herein. However, using the Ni²⁺/PPQN^{2,4-di-OMe} instead of its Brønsted acid salt analogues here, it was aimed to demonstrate its capability in oxidative, reductive and energy-transfer photocatalysis. Once these properties were confirmed and assuming Ni²⁺/PPQN^{2,4-di-OMe} behaved similarly to common bipyridyl nickel(II) transition metal catalysts, Ni²⁺/PPQN^{2,4-di-OMe} should, in principle, be able to manage the dual metallophotoredox cross-couplings as a singular entity.

The synthesis of the PPQN^{2,4-di-OMe} ligand was performed as per scheme 19 and explained above. The following procedure was applicable to all the couplings of aryl halides and benzyltrifluoroborates unless otherwise noted. The catalyst was synthesized by prestirring PPQN^{2,4-di-OMe} (3.4 mg, 10 μmol, 5.0 mol %) and NiCl₂·DME (2.2 mg, 10 μmol, 5.0 mol %) in CH₂Cl₂ (1.0 mL) in a 10 mL pyrex microwave tube for 30 min. The solvent was evacuated before aryl halide (0.20 mmol, 1.0 equiv), potassium benzyltrifluoroborate (0.30 mmol, 1.5 equiv), acetone (1.9 mL), MeOH (0.10 mL), and 2,6-lutidine (81 μL, 75.0 mg, 0.70 mmol, 3.5 equiv) were added (scheme 31). The tube was then sealed by a rubber septum, degassed by three freeze pump-thaw cycles, back-filled with argon, and stirred at room temperature under the 53 W 390 nm LED irradiation. After 20 h, the reaction mixture was passed through a short pad of silica gel and concentrated to afford the crude product. The product was purified by preparative thin layer chromatography. Unless otherwise specified, a 390 nm Kessil lamp was used as light source. The percent yield represents purified product unless otherwise specified.

Scheme 31 shows a generic reaction with an electrophile compound containing a halogen group X and a nucleophile containing a benzyl potassium trifluoroborate group. Different electrophiles and nucleophiles were tested as per scheme 31 and the yield results are shown in Table 16.

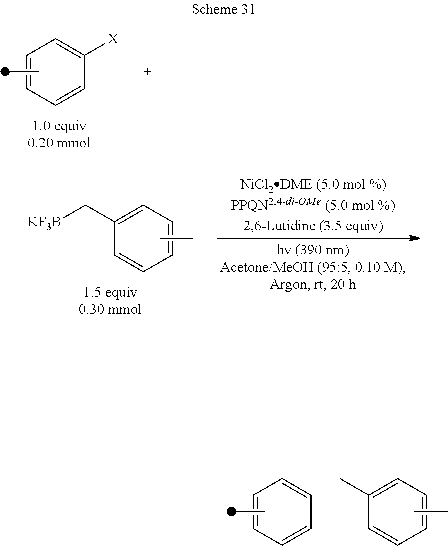

TABLE 16
Results of scheme 31 with different electrophiles and nucleophiles

| Entry | | | Product |
|---|---|---|---|
| # | Electrophile | Nucleophile | yield |
| 1 | 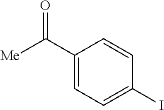 | Benzyl potassium tetrafluoroborate (BnBF₃K) | 75% |
| 2 | 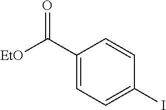 [SMILES: CCOC(=O)c1ccc(I)cc1] | BnBF₃K | 52% |
| 3 | 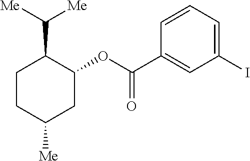 [SMILES: CC(C)[C@@H]1CC[C@@H](C)C[C@H]1OC(=O)c1cccc(I)c1] | BnBF₃K | 61% |
| 4 | 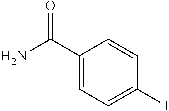 | BnBF₃K | 47% |
| 5 | 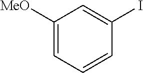 | BnBF₃K | 40% |
| 6 | 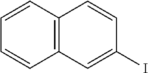 [SMILES: Ic1ccc2ccccc2c1] | BnBF₃K | 37% |
| 7 | 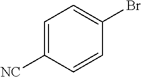 | BnBF₃K | 61% |
| 8 | 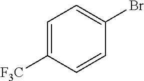 [SMILES: FC(F)(F)c1ccc(Br)cc1] | BnBF₃K | 59% |
| 9 | 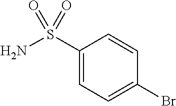 [SMILES: NS(=O)(=O)c1ccc(Br)cc1] | BnBF₃K | 53% |
| 10 | 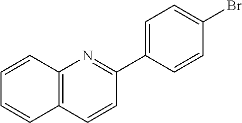 [SMILES: Brc1ccc(-c2ccc3ccccc3n2)cc1] | BnBF₃K | 44% |
| 11 | 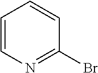 | BnBF₃K | 58% |
| 12 | 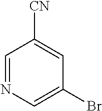 | BnBF₃K | 35% |
| 13 | 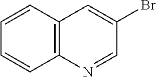 [SMILES: Brc1cnc2ccccc2c1] | BnBF₃K | 41% |
| 14 | 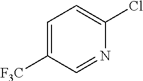 [SMILES: FC(F)(F)c1ccc(Cl)nc1] | BnBF₃K | 43% |
| 15 | 4-CN—C₆H₄—I | 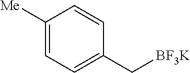 | 90% |
| 16 | 4-CN—C₆H₄—Br | 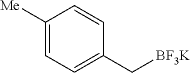 | 91% |
| 17 | 4-CN—C₆H₄—I | 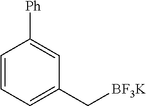 | 92% |
| 18 | 4-CN—C₆H₄—Br | 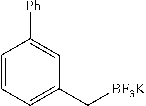 | 93% |
| 19 | 4-CN—C₆H₄—I | 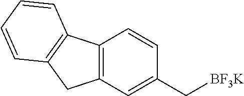 | 59% |
| 20 | 4-CN—C₆H₄—Br | 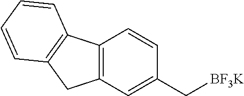 | 65% |
| 21 | 4-CN—C₆H₄—I | 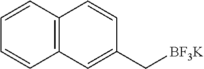 | 94% |
| 22 | 4-CN—C₆H₄—Br | 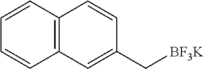 | 90% |
| 23 | 4-CN—C₆H₄—I | 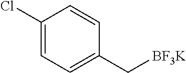 | 82% |
| 24 | 4-CN—C₆H₄—Br | 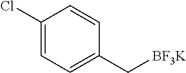 | 86% |
| 25 | 4-CN—C₆H₄—I | 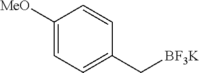 | 97% |
| 26 | 4-CN—C₆H₄—Br | 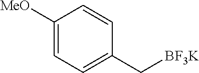 | 95% |
| 27 | 4-CN—C₆H₄—Br | 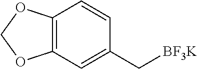 | 98% |
| 28 | 4-CN—C₆H₄—Br | 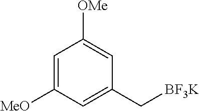 | 78% |
| 29 | 4-CN—C₆H₄—Br | 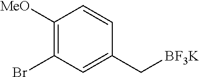 | 80% |
| 30 | 4-CN—C₆H₄—Br | 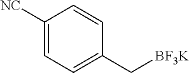 | 32% |
| 31 | 4-CN—C₆H₄—Br | 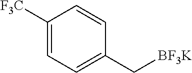 | 50% |
| 32 | 4-CN—C₆H₄—Br | 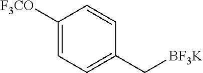 | 81% |
| 33 | 4-CN—C₆H₄—Br | 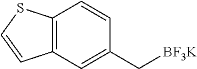 | 73% |

A site-selective SM cross-coupling was also accomplished (scheme 32) using the same reaction conditions as scheme 31, affording the mono-debrominated product (yield of 31%) as the paracyclophane precursor.

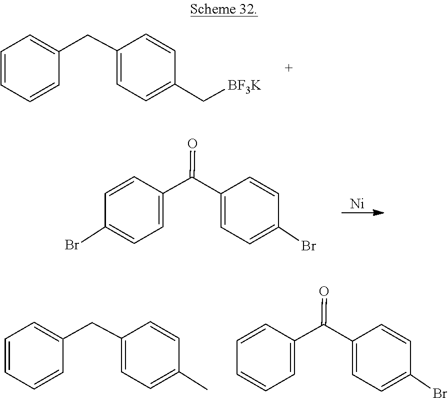

In general, (hetero)aryl electrophiles with electron-withdrawing (ketone, ester, amide, trifluoromethyl, sulfonamide, and nitrile) and -donating (methoxy) groups were all tolerated under the tested conditions (entries 1-14 in Table 16). The product yields of electron-deficient iodides (entries 1-4 in Table 16) outweighed the electron-rich (entry 5 in Table 16) and naphthyl ones (entry 6 in Table 16). Different aryl (entries 7-10 in Table 16) and heteroaryl (entries 11-13 in Table 16) bromides were proven effective coupling partners with the benzyl trifluoroborate, and so was the 2-pyridyl chloride (entry 14 in Table 16).

Regarding the R-BF₃K scope, various benzyltrifluoroborates, including those substituted by methyl (entries 15-16 in Table 16), p-extended (entries 17-22 in Table 16), chloro (entries 23-24 in Table 16), methoxy (entries 25-29 in Table 16), nitrile (entry 30 in Table 16), trifluoromethyl (entry 31 in Table 16), and trifluoromethoxy (entry 32 in Table 16) groups, were examined, delivering the diarylmethanes in moderate to excellent yields. In many cases, aryl bromides and iodides resulted in similar yields (entries 15-26 in Table 16). Noticeably, the bromo handle in entry 29 (Table 16) remained intact, setting the stage for iterative cross-couplings. In addition, trifluoroborate entry 33 (Table 16) with benzothiophene, a moiety found in bioactive structures, also afforded the expected product in high yield.

Various Ni/PPQN^{2,4-di-OMe}-catalysed metallophotoredox cross-couplings were tested. More specifically, redox-neutral C—C bond-forming reactions were attempted with the same Ni-based transformative platform as explained above. Unless otherwise specified the couplings were conducted on a 0.20 mmol scale, a 390 nm Kessil lamp was used as light source. The >25% yield represents purified product, and yield <25% refers to NMR yield with dibromomethane as internal standard.

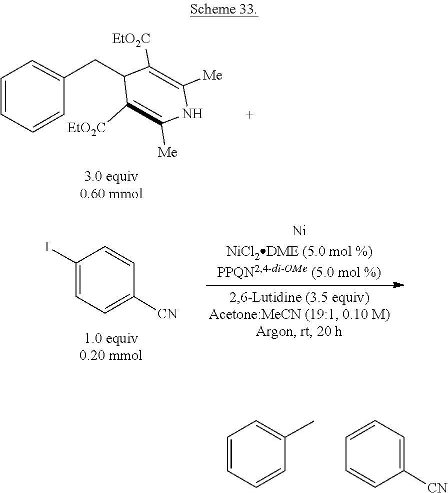

As per scheme 33 above, the catalyst was synthesized by pre-stirring PPQN^{2,4-di-OMe} (3.4 mg, 10 μmol, 5.0 mol %) and NiCl₂·DME (2.2 mg, 10 μmol, 5.0 mol %) in CH₂Cl₂ (1.0 mL) in a 10 mL pyrex microwave tube for 30 min. The solvent was evacuated before 4-iodobenzonitrile (45.8 mg, 0.20 mmol, 1.0 equiv), Hantzsch ester (189.0 mg, 0.60 mmol, 3.0 equiv), acetone (1.9 mL), MeOH (0.10 mL), and 2,6-lutidine (81 μL, 75.0 mg, 0.70 mmol, 3.5 equiv) were added. The tube was then sealed with a rubber septum, degassed by three freeze-pump-thaw cycles, back-filled with argon, and stirred at room temperature under the 53 W 390 nm LED irradiation. After 20 h, the reaction mixture was passed through a short pad of silica gel and concentrated to afford the crude product. The product was purified by preparative thin-layer chromatography. The yield obtained is shown in Table 17. The yields obtained for the control conditions: without transition metal, without ligand or without light are also shown in Table 17.

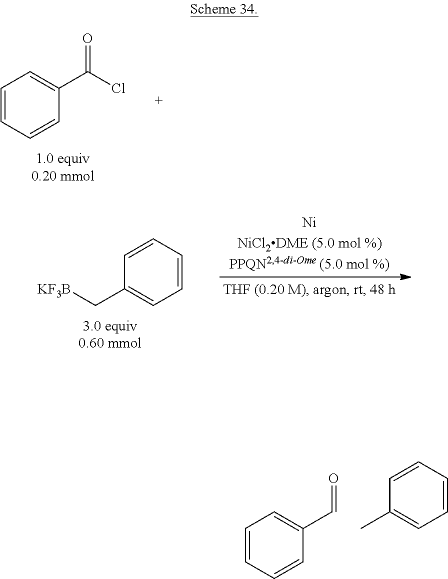

As per scheme 34 above, the catalyst was synthesized by pre-stirring PPQN^{2,4-di-OMe} (3.4 mg, 10 μmol, 5.0 mol %) and NiCl₂·DME (2.2 mg, 10 μmol, 5.0 mol %) in CH₂Cl₂ (1.0 mL) in a 10 mL pyrex microwave tube for 30 min. The solvent was evacuated before potassium benzyltrifluoroborate (119.0 mg, 0.60 mmol, 3.0 equiv) and tetrahydrofuran (THF) (1.0 mL) were added. The tube was sealed with a rubber septum, degassed by three freeze-pump-thaw cycles, and back-filled with argon. Benzoyl chloride (23.2 μL, 28.1 mg, 0.20 mmol, 1.0 equiv) was then added via a syringe. The reaction mixture was stirred at room temperature under the 53 W 390 nm LED irradiation. After 20 h, the reaction mixture was passed through a short pad of silica gel and concentrated to afford the crude product. The product was purified by preparative thin-layer chromatography. The yield obtained is shown in Table 17. The yields obtained for the control conditions: without transition metal, without ligand or without light are also shown in Table 17.

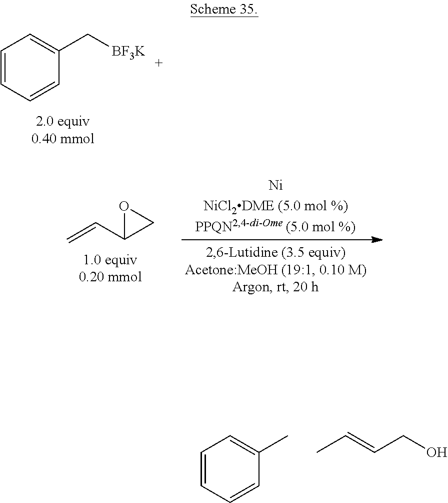

As per scheme 35 above, the catalyst was synthesized by pre-stirring PPQN^{2,4-di-OMe} (3.4 mg, 10 μmol, 5.0 mol %) and NiCl₂·DME (2.2 mg, 10 μmol, 5.0 mol %) in CH₂Cl₂ (1.0 mL) in a 10 mL pyrex microwave tube for 30 min. The solvent was evacuated before butadiene monoxide (16.2 μL, 14.0 mg, 0.20 mmol, 1.0 equiv), potassium benzyltrifluoroborate (79.2 mg, 0.40 mmol, 2.0 equiv), acetone (1.9 mL), and MeOH (0.10 mL), and 2,6-lutidine (81 μL, 75.0 mg, 0.70 mmol, 3.5 equiv) were added. The tube was sealed with an aluminium cap with a septum, degassed by three freeze-pump-thaw cycles, back-filled with argon, and stirred at room temperature under the 53 W 390 nm LED irradiation. After 20 h, the reaction mixture was passed through a short pad of silica gel and concentrated to afford the crude product. The product was purified by preparative thin-layer chromatography. The yield obtained is shown in Table 17. The yields obtained for the control conditions: without transition metal, without ligand or without light are also shown in Table 17.

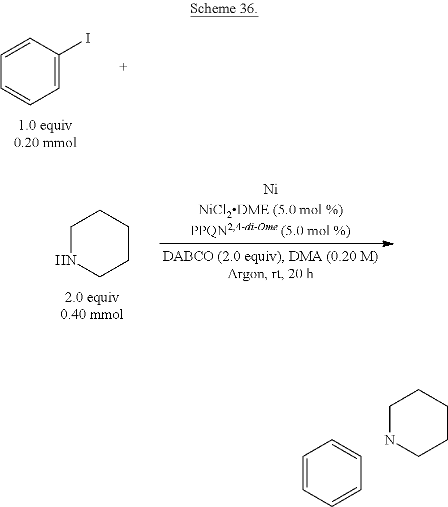

As per scheme 36 above, the catalyst was synthesized by pre-stirring PPQN^{2,4di-OMe} (3.4 mg, 10 μmol, 5.0 mol %) and NiCl₂·DME (2.2 mg, 10 μmol, 5.0 mol %) in N,N-dimethylacetamide (DMA, 1.0 mL) in a 10 mL pyrex microwave tube for 30 min. The solvent was evacuated before iodobenzene (40.8 mg, 0.20 mmol, 1.0 equiv), piperidine (39 μL, 34.0 mg, 0.40 mmol, 2.0 equiv), and 1,4-diazabicyclo[2.2.2]octane (DABCO, 44.9 mg, 0.40 mmol, 2.0 equiv) were added. The tube was sealed with an aluminium cap with a septum, degassed by three freeze-pump-thaw cycles, back-filled with argon, and stirred at room temperature under the 53 W 390 nm LED irradiation. After 20 h, the reaction mixture was passed through a short pad of silica gel and concentrated to afford the crude product. The product was purified by preparative thin-layer chromatography. The yield obtained is shown in Table 17. The yields obtained for the control conditions: without transition metal, without ligand or without light are also shown in Table 17.

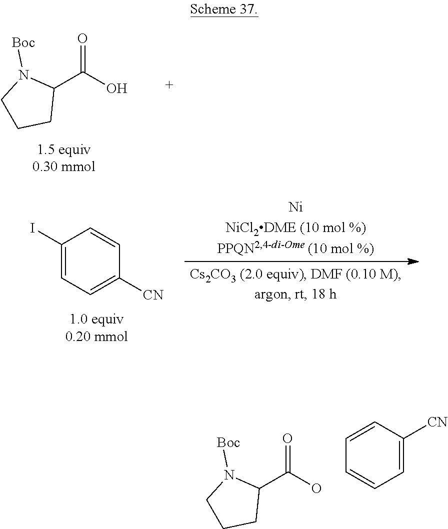

As per scheme 37 above, the catalyst was synthesized by pre-stirring PPQN^{2,4-di-OMe} (6.8 mg, 20 μmol, 10 mol %) and NiCl₂·DME (4.4 mg, 20 μmol, 10 mol %) in DMF (2.0 mL) in a 10 mL pyrex microwave tube for 30 min. 4-lodobenzonitrile (45.8 mg, 0.20 mmol, 1.0 equiv), Boc-Pro-OH (37.6 mg, 0.30 mmol, 1.5 equiv) and Cs₂CO₃ (130.0 mg, 0.40 mmol, 2.0 equiv) were then added. The tube was then sealed with a rubber septum, degassed by three freeze-pump-thaw cycles, back-filled with argon, and stirred at room temperature under the 53 W 390 nm LED irradiation. After 20 h, to the reaction mixture was added brine, which was extracted with EtOAc, filtered through a short pad of MgSO₄, and concentrated to afford the crude product. The product was purified by preparative thin-layer chromatography. The yield obtained is shown in Table 17. The yields obtained for the control conditions: without transition metal, without ligand or without light are also shown in Table 17.

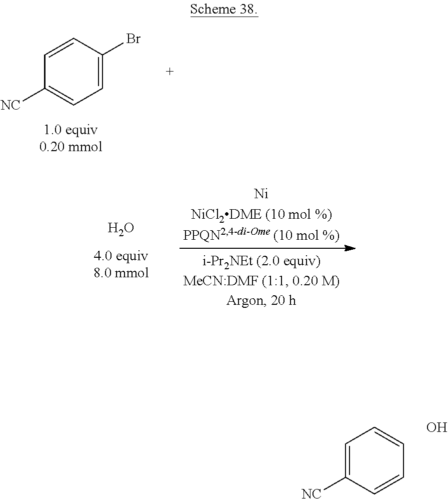

As per scheme 38 above, the catalyst was synthesized by pre-stirring PPQN^{2,4-di-OMe} (6.8 mg, 20 μmol, 10 mol %) and NiCl₂·DME (4.4 mg, 20 μmol, 10 mol %) in DMF (0.50 mL) in a 10 mL pyrex microwave tube for 30 min. 4-Bromobenzonitrile (36.4 mg, 0.20 mmol, 1.0 equiv), H₂O (144 μL, 144.0 mg, 8.0 mmol, 40 equiv), i-Pr₂NEt (N,N-Diisopropylethylamine, 70 μL, 51.7 mg, 0.40 mmol, 2.0 equiv), and MeCN (0.50 mL) were then added. The tube was then sealed with a rubber septum, degassed by three freeze-pump-thaw cycles, back-filled with argon, and stirred under the 53 W390 nm LED irradiation. After 20 h, to the reaction mixture was added brine, which was extracted with EtOAc, filtered through a short pad of MgSO₄, and concentrated to afford the crude product. The yield obtained is shown in Table 17. The yields obtained for the control conditions: without transition metal, without ligand or without light are also shown in Table 17.

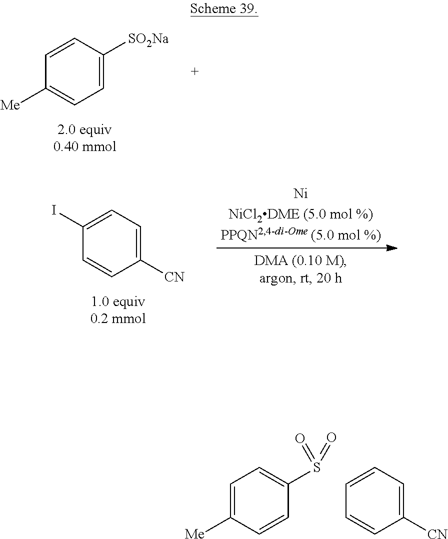

As per scheme 39 above, the catalyst was synthesized by pre-stirring PPQN^{2,4-di-OMe} (3.4 mg, 10 μmol, 5.0 mol %) and NiCl₂·DME (2.2 mg, 10 μmol, 5.0 mol %) in DMA (2.0 mL) in a 10 mL pyrex microwave tube for 30 min. 4-lodobenzonitrile (46.0 mg, 0.20 mmol, 1.0 equiv) and sodium ptoluenesulfinate (TsSO₂Na, 71.0 mg, 0.40 mmol, 2.0 equiv) were then added. The tube was then sealed with a rubber septum, degassed by three freeze-pump-thaw cycles, back-filled with argon, and stirred at room temperature under the 53 W 390 nm LED irradiation. After 20 h, to the reaction mixture was added brine, which was extracted with EtOAc, filtered through a short pad of MgSO₄, and concentrated to afford the crude product. The product was purified by preparative thin-layer chromatography. The product was purified by preparative thin-layer chromatography. The yield obtained is shown in Table 17. The yields obtained for the control conditions: without transition metal, without ligand or without light are also shown in Table 17.

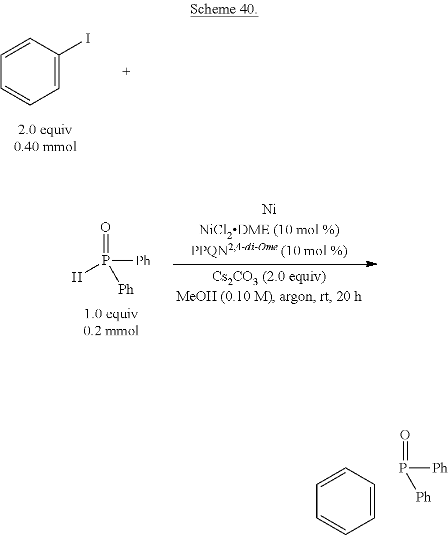

As per scheme 40 above, the catalyst was synthesized by pre-stirring PPQN^{2,4-di-OMe} (6.8 mg, 20 μmol, 10 mol %) and Ni(PPh₃)₂Cl₂ (13.0 mg, 20 μmol, 10 mol %) in MeOH (1.0 mL) in a 10 mL pyrex microwave tube for 30 min. Diphenylphosphine oxide (40.4 mg, 0.20 mmol, 1.0 equiv), iodobenzene (44 μL, 81.6 mg, 0.40 mmol, 2.0 equiv), and Cs2CO3 (130.4 mg, 0.40 mmol, 2.0 equiv) were added. The tube was sealed with a rubber septum, degassed by three freeze-pump-thaw cycles, and back-filled with argon. The reaction mixture was stirred at room temperature under the 53 W 390 nm LED irradiation. After 20 h, the reaction mixture was passed through a short pad of silica gel and concentrated to afford the crude product. The product was purified by preparative thin-layer chromatography. The yield obtained is shown in Table 17. The yields obtained for the control conditions: without transition metal, without ligand or without light are also shown in Table 17.

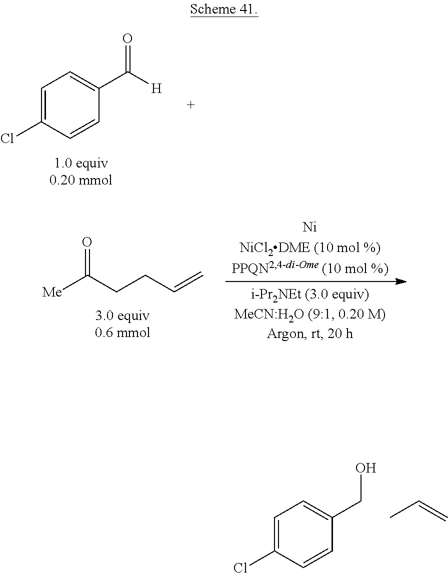

As per scheme 41, the catalyst was synthesized by pre-stirring PPQN^{2,4-di-OMe} (6.8 mg, 20 μmol, 10 mol %) and NiCl₂·DME (4.4 mg, 20 μmol, 10 mol %) in CH₂Cl₂ (1.0 mL) in a 10 mL pyrex microwave tube for 30 min. The solvent was evacuated before 4-chlorobenzaldehyde (28.2 mg, 0.20 mmol, 1.0 equiv), allyl acetate (64 μL, 60.0 mg, 0.60 mmol, 3.0 equiv), i-Pr₂Net (104 μL, 77.6 mg, 0.60 mmol, 3.0 equiv), MeCN (0.90 mL), and H₂O (0.10 mL) were added. The tube was sealed with a rubber septum, degassed by three freeze-pump-thaw cycles, and backfilled with argon. The reaction mixture was stirred at room temperature under the 53 W 390 nm LED irradiation. After 20 h, the reaction mixture was passed through a short pad of MgSO₄ and concentrated to afford the crude product. The product was purified by preparative thin-layer chromatography. The yield obtained is shown in Table 17. The yields obtained for the control conditions: without transition metal, without ligand or without light are also shown in Table 17.

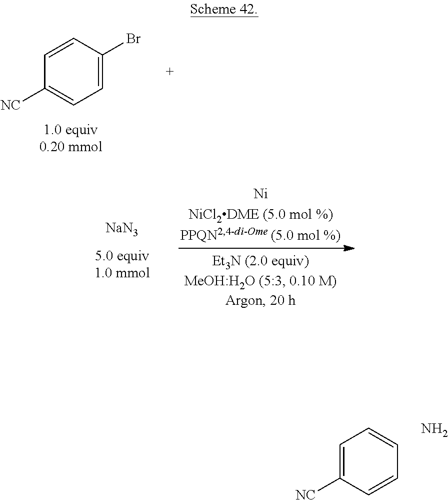

As per scheme 42 above, the catalyst was synthesized by pre-stirring PPQN^{2,4-di-OMe} (3.4 mg, 10 μmol, 5.0 mol %) and NiCl₂·DME (2.2 mg, 10 μmol, 5.0 mol %) in CH₂Cl₂ (1.0 mL) in a 10 mL pyrex microwave tube for 30 min. The solvent was evacuated before 4-bromobenzonitrile (36.4 mg, 0.20 mmol, 1.0 equiv), NaN₃ (65.0 mg, 1.0 mmol, 5.0 equiv), Et3N (28 μL, 20.2 mg, 0.40 mmol, 2.0 equiv), followed by MeOH (1.25 mL) and H₂O (0.75 mL), were added. The tube was sealed with a rubber septum, degassed by three freeze-pump-thaw cycles, and back-filled with argon. The reaction mixture was stirred under the 53 W 390 nm LED irradiation. After 20 h, the reaction mixture was passed through a short pad of silica gel and concentrated to afford the crude product. The product was purified by preparative thin-layer chromatography. The yield obtained is shown in Table 17. The yields obtained for the control conditions: without transition metal, without ligand or without light are also shown in Table 17.

TABLE 17
Yields obtained for the reactions of schemes 33-42

| | | Control, without | Control, without | Control, without |
|---|---|---|---|---|
| Reaction | Standard | transition metal | ligand | light |
| Scheme 33 | 85% | 0% | 0% | 0% |
| Scheme 34 | 41% | 0% | 0% | 0% |
| Scheme 35 | 52% | 0% | 0% | 0% |
| Scheme 36 | 53% | 0% | 8% | 0% |
| Scheme 37 | 55% | 0% | 0% | 0% |
| Scheme 38 | 90% | 0% | 23% | 0% |
| Scheme 39 | 62% | 0% | 0% | 0% |
| Scheme 40 | 51% | 0% | 16% | 0% |
| Scheme 41 | 62% | 0% | 0% | 0% |
| Scheme 42 | 56% | 12% | 18% | 0% |

In place of RBF₃K, Hantzsch's ester, which was derived from the corresponding aldehydes, was also shown as an efficacious radical source for the Ni-catalyzed metallophotoredox C(sp³)-C(sp²) cross-coupling (scheme 33). Under light irradiation, aroyl chloride and alkenyl epoxide were found compatible with the nickel photocatalysis, extending the electrophile scope and giving aryl alkyl ketone and allylic alcohol as desired products (schemes 34 and 35).

Satisfactorily, Ni²⁺/PPQN^{2,4-di-OMe}-catalyzed C—X bond formation was amenable by pairing some heteroatomic nucleophiles with various aromatic halides. In this category, Ni²⁺/PPQN^{2,4-di-OMe} enabled the photoamination of unactivated aryl iodide with an aliphatic amine in a good yield (scheme 36), although electronically biased aryl halides were frequently needed in known metallophotoredox C—N cross-couplings. Encouragingly, phenol and its derivatives were obtained under mild conditions from the coupling reactions with O-nucleophiles, such as carboxylic acid and water (schemes 37-38). Harsh conditions such as strong bases and elevated temperatures were often required for the same synthetic purposes. C(sp²)-S and C(sp²)-P bond formation was feasible by harnessing the “two-in-one” Ni—PC, providing diarylsulfone and triarylphosphine via Ullmann-type couplings (schemes 39-40).

Moreover, the catalytic versatility of Ni²⁺/PPQN^{2,4-di-OMe} reached beyond redoxneutral transformations, enabling a Nozaki-Hiyama-Kishi (NHK)-type cross-electrophile coupling between aldehyde and allylic ester with tertiary amine as the organic sacrificial reductant (scheme 41). The reductive aromatic amination with azide as the N-source also proceeded efficiently with the Ni-metallophotocatalyst (scheme 42). Due to the high value of primary anilines and the lack of general metallophotoredox protocols to access themvia cross-couplings, the present catalytic method is a valuable addition to the primary aniline synthesis toolbox. It is to be noted that, in schemes 33-42, an inconsequential quantity of products were observed if the metal, ligand, or light was omitted (control conditions, Table 17).

The use of Zn instead of Ni was tested in a metallophotoredox Suzuki coupling with Zn²⁺/PPQN^{2,4-di-OMe}. Zn²⁺/PPQN^{2,4-di-OMe} was made by pre-stirring equimolar ZnCl₂·1,2-dimethoxyethane (DME) and PPQN^{2,4-di-OMe} and a 390 nm Kessil™ lamp was used as light source. The metallophotoredox Suzuki coupling was performed as per Scheme 43 shown below. The no product was obtained (yield of 0%).

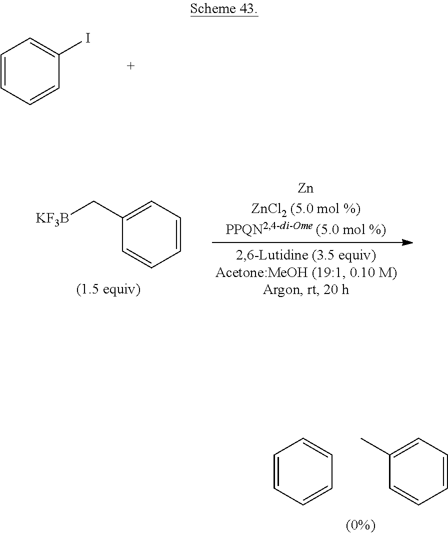

The reactions of schemes 33-42 were repeated with the same conditions but Zn²⁺/PPQN^{2,4-di-OMe} was used instead of Ni²⁺/PPQN^{2,4-di-OMe}. No product was obtained (yield of 0%) except for the reaction of scheme 34 were a 6% yield was obtained. In all the Ni-catalysed metallophotoredox cross-couplings tested herein, zinc was proven inefficient, indicating the transition-metal-catalysed redox chemistry was only viable in the presence of redox-active metals like nickel. In the dark, all the redox-neutral C—C and C—X couplings and reductive cross-electrophile C—C coupling did not proceed, showing the indispensable role of photoexcitation in these transformations. Accordingly, the product formation was significantly inhibited when replacing Ni²⁺ with redox-innocent Zn²⁺. The present results thus illustrate the essential cooperation between the redox-active Ni²⁺/PPQN^{2,4-di-OMe} and light excitation, which did not only simplify the conditions of Ni-metallophotoredox cross-couplings but also broadened the ground-state chemistries of nickel catalysis.

To elucidate some mechanistic underpinnings of Ni²⁺/PPQN^{2,4-di-OMe} metallaphotocatalysis, spectroscopic analysis and computational calculations were performed. All computations were performed using linear response time-dependent DFT (TD-DFT) with the 6-311G* basis set in a Gaussian™ 16 software package.

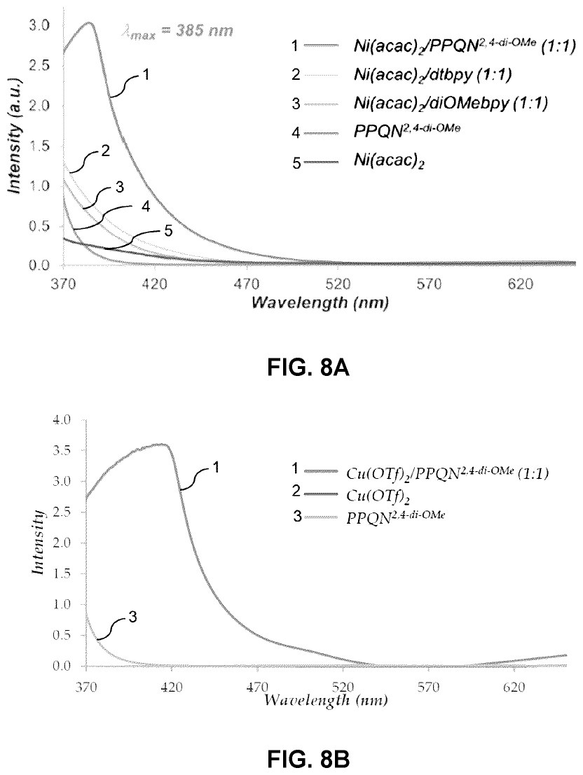

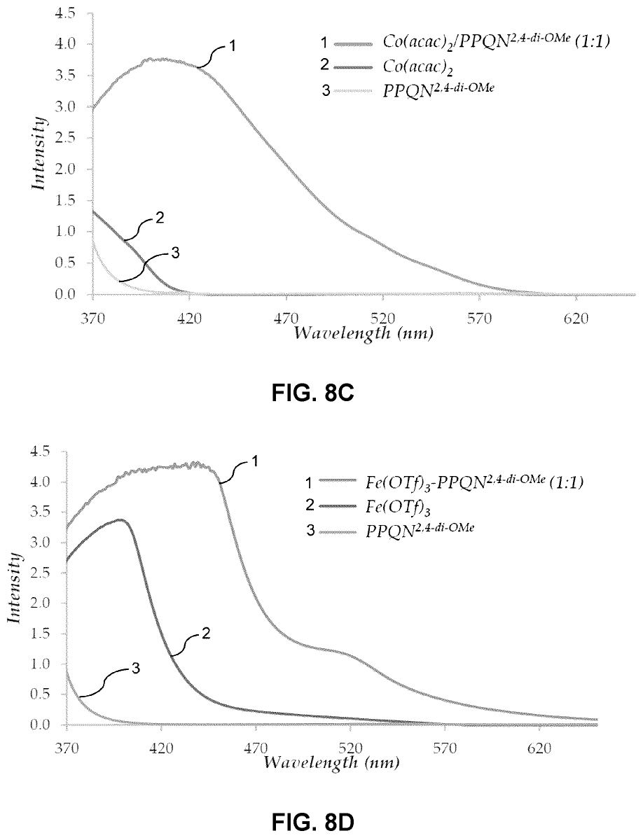

First, Ni²⁺/PPQN^{2,4-di-OMe} was characterized by ultraviolet-visible (UV-vis) spectroscopy, which showed a prominent absorption peak (λₘₐₓ=385 nm) of violet and blue light and overlapped consistently with the emission spectrum of 390 nm Kessil lamp (FIG. 8A). The solutions were prepared with 0.050 mmol substrates and degassed solvents in 10 mL volumetric flasks. For metal-PPQN^{2,4-di-OMe} complexes, 0.050 mmol of a metal salt and PPQN^{2,4-di-OMe} were mixed and stirred in 2.0 mL solvent (hexamethylphosphoramide (HMPA)) for 2.0 h before being diluted to 10.0 mL. The final concentrations were set to be 5.0 mM thereby. Copper in the form of Copper(II) trifluoromethanesulphonate (Cu(OTO₂), cobalt in the form of Co(acac)₂, and iron in the form of Fe(OTf)₃ were tested (respectively FIGS. 8B, 8C and 8D).

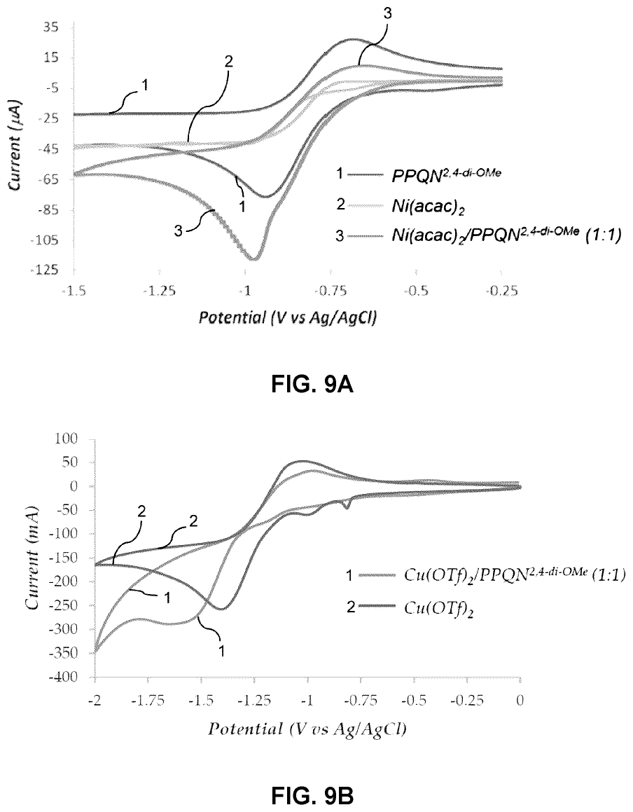

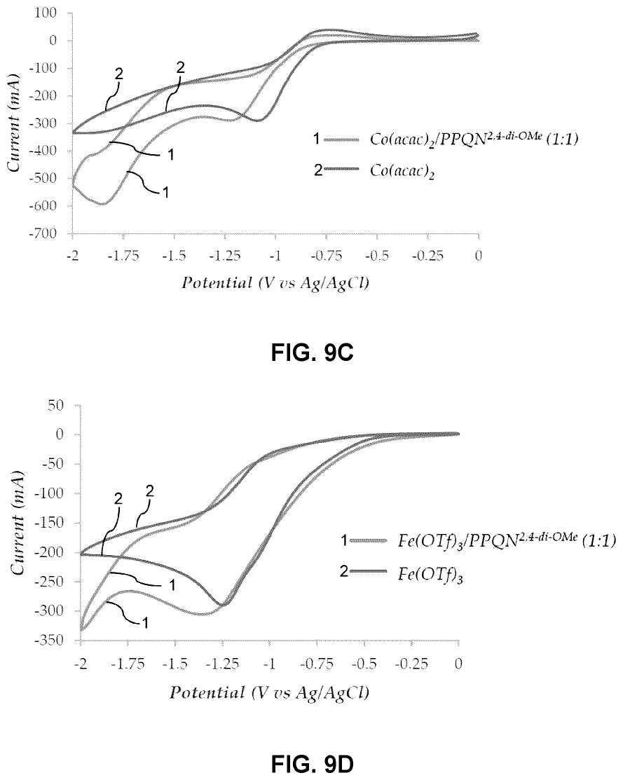

In line with the control experiments, Ni²⁺, free PPQN^{2,4-di-OMe}, as well as the Ni²⁺/dtbpy and Ni²⁺/diOMebpy counterparts were of weak absorptivity in the same region. Cyclic voltammetry (CV) featured distinct electrochemical patterns of Ni²⁺/PPQN^{2,4-di-OMe} relative to its metal and ligand components (FIG. 9A). All the electrochemical experiments were performed with HEKA™ PG 340 potentiostat with Ag/AgCl as the reference electrode. The working electrode was made of glassy carbon, and a Pt wire was used as the counter electrode to complete the electrochemical setup. A scan rate of 100 mV/s was used for all experiments. All the potentials were noted with respect to the Ag/AgCl electrode unless otherwise specified. The measurement of Ni(acac)₂ was used as an example (Ni(acac)₂ was used for better solubility instead of NiCl₂·DME). A 50 mL beaker was charged with Ni(acac)₂) (5.1 mg, 0.020 mmol, 1.0 mM), tetrabutylammonium hexafluorophosphate (Bu₄NPF₆, 774.9 mg, 2.0 mmol, 0.10 M), and 20.0 mL degassed HPLC-grade MeCN. After stirring for a while, the homogeneous solution was subjected to the cyclic voltammetric measurement (for Ni(acac)₂-PPQN^{2,4-di-OMe}, the solution was pre-stirred vigorously for 1.0 h before the measurement). Same procedures were adopted for the PPQN^{2,4-di-OMe} complexes of copper, cobalt and iron. For Cu, Cu(OTf₂/PPQN^{2,4-di-OMe} 1.0 m, and Cu(OTf)₂ with 0.10 M Bu₄NPF₆ in MeCN were tested (FIG. 9B). For Co, Co(acac)₂/PPQN^{2,4-di-OMe} 1.0 mM and Co(acac)₂ with 0.10 M Bu₄NPF₆ in MeCN were tested (FIG. 9C). For Fe, Fe(OTf)₃/PPQN^{2,4-di-OMe} 1.0 mM and Fe(OTf)₃ with 0.10 M Bu₄NPF₆ in MeCN were tested (FIG. 9D).

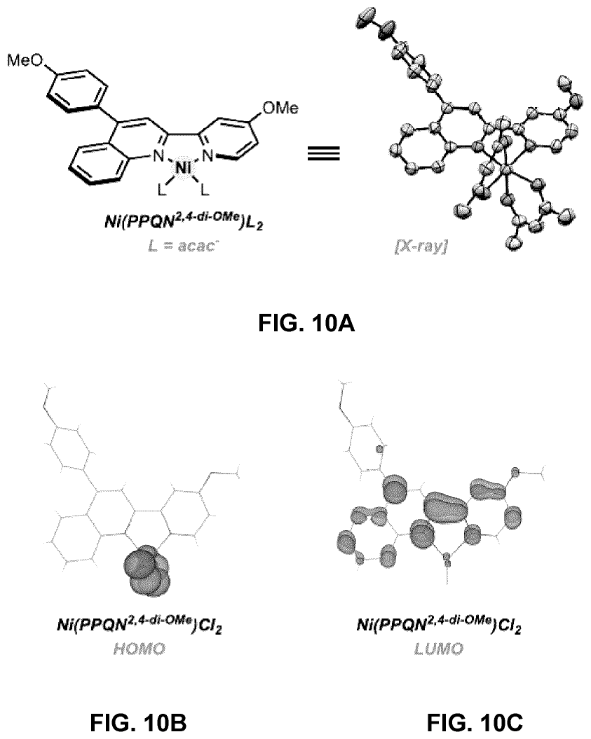

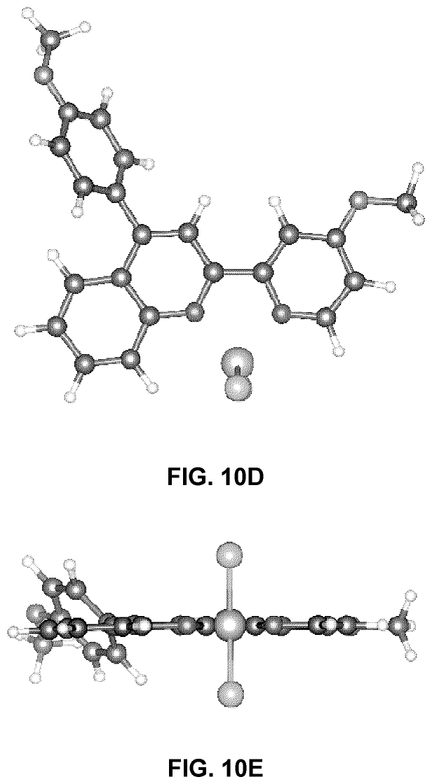

Additionally, the coordination between Ni²⁺ and PPQN^{2,4-di-OMe} was ascertained by the solid-state structure of their complex (FIG. 10A). Then, time-dependent-density functional theory (TDDFT) was used to compute the electronic structures of the model complex, Ni(PPQN^{2,4-di-OMe})Cl₂. As a result, computation depicted a delocalized ligand-Tr*-centered lowest unoccupied orbital (FIGS. 10B-10C). Such a configuration resembled those precious metal polypyridyl PCs and supported the initial design of Ni-photoredox catalyst.

The ground state geometry was optimised using DFT, and the excited states were calculated with linear response time-dependent DFT (TDDFT) at the optimised ground state geometry. All calculations were performed with the Gaussian™ 16 package (Rev. C.01) using the PBEO functional and the 6-311G* basis set. Grimme's D3BJ dispersion correction was used to improve calculation accuracy. The optimised structures of Ni(PPQN^{2,4-di-OMe})Cl₂ are shown in FIGS. 10D and 10E, top view and front view respectively, and Table 18 below shows the energy for the orbitals.

TABLE 18
Summary of the energies for each orbital calculated

| Orbital | label | Energy |
|---|---|---|
| Orbital 122 | α-HOMO | −6.683788 eV |
| Orbital 740 | β-HOMO | −6.434968 eV |
| Orbital 123 | α-LUMO | −2.751196 eV |
| Orbital 741 | β-LUMO | −2.711818 eV |

The merging PPQN^{2,4-di-OMe} and earth-abundant first-row metals such as iron, cobalt, and copper enriches the base-metal photochemistry and brings more fruitful transformation reactions. This was demonstrated in schemes 44-48 shown below and the yields are summarized in Table 19 below.

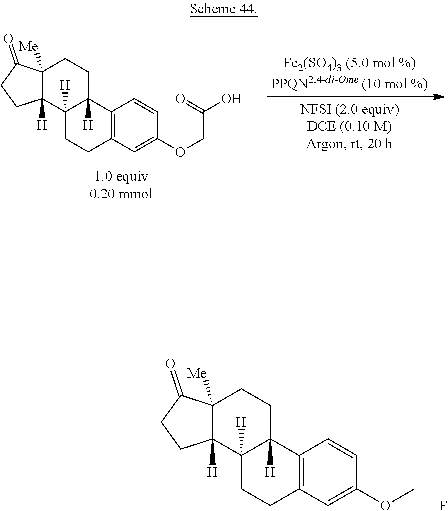

The catalyst was synthesized by pre-stirring PPQN^{2,4-di-OMe} (6.8 mg, 20 μmol, 10 mol %) and Fe₂(SO₄)₃(4.0 mg, 10 μmol, 5.0 mol %) in 1,2-dichloroethane (DCE) (2.0 mL) in a 10 mL pyrex microwave tube for 30 min. Carboxylic acid (65.6 mg, 0.20 mmol, 1.0 equiv) and N-fluorobenzenesulfonimide(NFSI, 126 mg, 0.40 mmol, 2.0 equiv) were added. The tube was sealed with a rubber septum, degassed by three freeze-pump-thaw cycles, and back-filled with argon. The reaction mixture was stirred at room temperature under the 53 W 390 nm LED irradiation. After 20 h, the reaction mixture was passed through a short pad of silica gel and concentrated to afford the crude product. The product was purified by preparative thin-layer chromatography. The yield obtained is shown in Table 19. The yields obtained for the control conditions: without transition metal, without ligand or without light are also shown in Table 19. As shown in scheme 44, the combination of the PPQN^{2,4-di-OMe} and simple ferric salt promoted the decarboxylative fluorination of the estrone-derived carboxylic acid which exemplified a convenient route to prepare the valuable monofluoromethoxylated product.

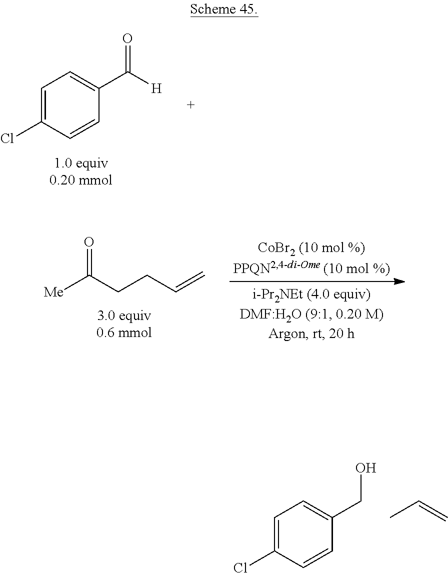

The catalyst was synthesized by pre-stirring PPQN^{2,4-di-OMe} (6.8 mg, 20 μmol, 10 mol %) and CoBr₂ (4.4 mg, 20 μmol, 10 mol %) in DMF (0.90 mL) in a 10 mL pyrex microwave tube for 30 min. 4-Chlorobenzaldehyde (28.2 mg, 0.20 mmol, 1.0 equiv), allyl acetate (64 μL, 60.0 mg, 0.60 mmol, 3.0 equiv), i-Pr₂NEt (104 μL, 77.6 mg, 0.60 mmol, 3.0 equiv), and H₂O (0.10 mL) were added. The tube was sealed with a rubber septum, degassed by three freeze-pump-thaw cycles, and back-filled with argon. The reaction mixture was stirred at room temperature under the 53 W 390 nm LED irradiation. After 20 h, to the reaction mixture was added brine, which was extracted with EtOAc, filtered through a short pad of MgSO₄, and concentrated to afford the crude product. The product was purified by preparative thin-layer chromatography. The yield obtained is shown in Table 19. The yields obtained for the control conditions: without transition metal, without ligand or without light are also shown in Table 19. Analogous to the Ni metallaphotocatalysis, the Co²⁺/PPQN2,4-di-OMe also derived the reductive allylation of the aldehyde with the allyl ester in the presence of tertiary amine (scheme 45), providing more flexibility for the retrosynthetic planning of allylic alcohol preparation. In tandem with PPQN^{2,4-di-OMe}, copper was also catalytically viable for several metallaphotoredox reactions.

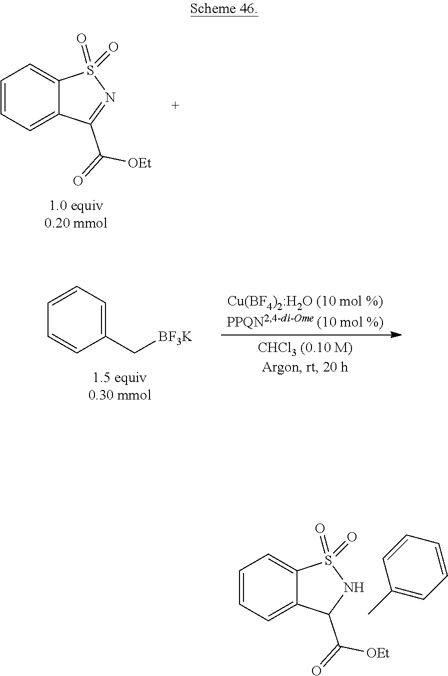

The catalyst was synthesised by pre-stirring PPQN^{2,4-di-OMe} (6.8 mg, 20 μmol, 10 mol %) and Cu(BF₄)₂·H₂O (5.2 mg, 20 μmol, 10 mol %) in MeCN (2.0 mL) in a 10 mL pyrex microwave tube for 30 min. N-Sulfonyl imine (47.8 mg, 0.20 mmol, 1.0 equiv) and potassium benzyltrifluoroborate (59.4 mg, 0.30 mmol, 1.5 equiv) were added. The tube was sealed with a rubber septum, degassed by three freeze-pump-thaw cycles, and back-filled with argon. The reaction mixture was stirred at room temperature under the 53 W 390 nm LED irradiation. After 20 h, the reaction mixture was passed through a short pad of silica gel and concentrated to afford the crude product. The product was purified by preparative thin-layer chromatography. The yield obtained is shown in Table 19. The yields obtained for the control conditions: without transition metal, without ligand or without light are also shown in Table 19.

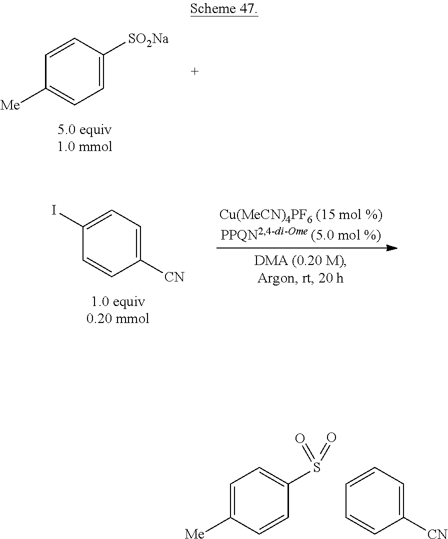

The catalyst was synthesized by pre-stirring PPQN^{2,4-di-OMe} (3.4 mg, 10 μmol, 5.0 mol %) and Cu(MeCN)₄BF₄ (11.2 mg, 30 μmol, 15 mol %) in DMA (1.0 mL) in a 10 mL pyrex microwave tube for 30 min. 4-lodobenzonitrile (45.8 mg, 0.10 mmol, 1.0 equiv) and sodium p-toluenesulfinate (TsSO₂Na, 178.2 mg, 1.0 mmol, 5.0 equiv) were added. The tube was then sealed with a rubber septum, degassed by three freeze-pump-thaw cycles, back-filled with argon, and stirred at room temperature under the 53 W 390 nm LED irradiation. After 20 h, to the reaction mixture was added brine, which was extracted with EtOAc, filtered through a short pad of MgSO₄, and concentrated to afford the crude product. The product was purified by preparative thin-layer chromatography. The yield obtained is shown in Table 19. The yields obtained for the control conditions: without transition metal, without ligand or without light are also shown in Table 19.

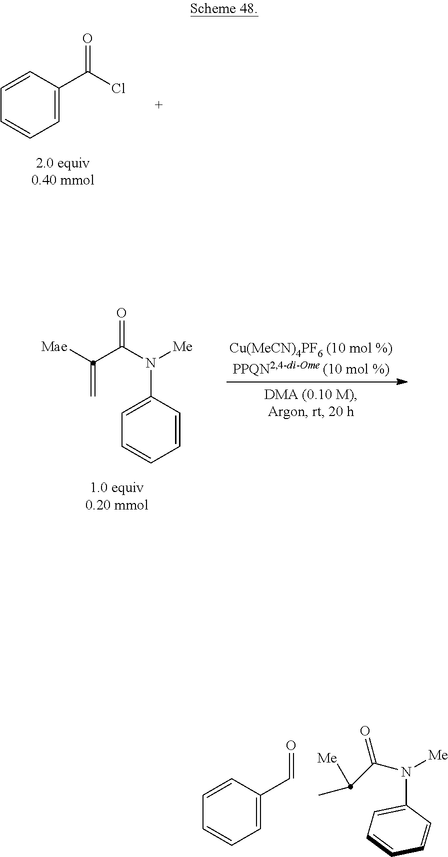

The catalyst was synthesized by pre-stirring PPQN^{2,4-di-OMe} (6.8 mg, 20 μmol, 10 mol %) and Cu(MeCN)₄PF₆ (7.4 mg, 20 μmol, 10 mol %) in DMA (2.0 mL) in a 10 mL pyrex microwave tube for 30 min. N-Methyl-N-phenylmethacrylamide (35.0 mg, 0.20 mmol, 1.0 equiv) was added. The tube was sealed with a rubber septum, degassed by three freeze-pumpthaw cycles, and back-filled with argon. Benzoyl chloride (46.4 μL, 28.1 mg, 0.40 mmol, 2.0 equiv) was then added via a syringe. After 20 h, to the reaction mixture was added brine, which was extracted with EtOAc, filtered through a short pad of MgSO4, and concentrated to afford the crude product. The product was purified by preparative thin-layer chromatography. The yield obtained is shown in Table 19. The yields obtained for the control conditions: without transition metal, without ligand or without light are also shown in Table 19.

With a common Cu(I) source (Cu(MeCN)₄BF₄), the Cu⁺/PPQN^{2,4-di-OMe}-mediated radical addition to imine (scheme 46), aromatic sulfonylation (scheme 47) as well as alkene dicarbofunctionalization (scheme 48) was performed successfully, which furnished the desired products accordingly (schemes 46-48).

TABLE 19
Yields obtained for the reactions of schemes 44-48

| | | | Control, | Control, |
|---|---|---|---|---|
| | | Control, without | without | without |
| Reaction | Standard | transition metal | ligand | light |
| Scheme 44 | 45% | 15% | 0% | 0% |
| Scheme 45 | 53% | 0% | 0% | 0% |
| Scheme 46 | 61% | 7% | 0% | 0% |
| Scheme 47 | 52% | 0% | 30% | 0% |
| Scheme 48 | 59% | 0% | 0% | 0% |

The NMR characterization of the compounds synthesized in the present Example are presented in Table 20.

TABLE 20
NMR characterization of compounds

| Compound | Characterization |
|---|---|
| 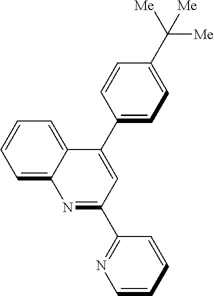 | White solid. ¹H NMR (500 MHz, CDCl₃) δ 8.77-8.73 (m, 1H), 8.71 (d, J = 7.9 Hz, 1H), 8.55 (s, 1H), 8.27 (d, J = 7.5 Hz, 1H), 8.05 (dd, J = 8.4, 1.7 Hz, 1H), 7.93-7.87 (m, 1H), 7.79- 7.73 (m, 1H), 7.58 (s, 4H), 7.55-7.50 (m, 1H), 7.40-7.35 (m, 1H), 1.44 (s, 9H). ¹³C NMR (126 MHz, CDCl₃) δ 156.5, 155.7, 151.4, 149.3, 149.2, 148.6, 136.9, 135.5, 130.2, 129.4, 129.3, 126.9, 126.7, 126.0, 125.4, 124.0, 121.9, 119.3, 34.8, 31.4. HRMS (M + H⁺) for C₂₄H₂₃N₂; Calculated: 339.1856, measured: 339.1658. |
| 4-(4-(Tert-butyl)phenyl)-2-(pyridin-2-yl)quinoline | |
| (PPQN^{4-t-Bu}) | |
| 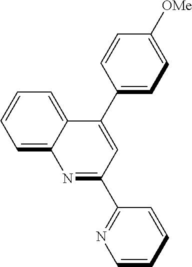 | White solid. ¹H NMR (500 MHz, CDCl₃) δ 8.78-8.73 (m, 1H), 8.71 (d, J = 7.9 Hz, 1H), 8.52 (s, 1H), 8.29-8.22 (m, 1H), 8.02 (dd, J = 8.4, 2.0 Hz, 1H), 7.94-7.87 (m, 1H), 7.79- 7.73 (m, 1H), 7.57 (d, J = 8.9 Hz, 2H), 7.55- 7.51 (m, 1H), 7.40-7.36 (m, 1H), 7.09 (d, J = 8.9 Hz, 2H), 3.94 (s, 3H). ¹³C NMR (126 MHz, CDCl₃) δ 159.8, 156.5, 155.6, 149.2, 149.0, 148.6, 137.0, 131.0, 130.7, 130.2, 129.3, 127.0, 126.7, 125.9, 124.0, 121.9, 119.2, 114.0, 55.4. HRMS (M + H⁺) for C₂₁H₁₇N₂O; Calculated: 313.1335, measured: 313.1331. |
| 4-(4-Methoxyphenyl)-2-(pyridin-2-yl)quinoline | |
| (PPQN^{4-OMe}) | |
| 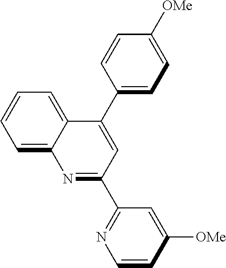 | White solid. ¹H NMR (500 MHz, CDCl₃) δ 8.56 (d, J = 5.5 Hz, 1H), 8.51 (s, 1H), 8.31-8.21 (m, 2H), 8.02 (dd, J = 8.4, 1.8 Hz, 1H), 7.82- 7.71 (m, 1H), 7.57 (d, J = 8.7 Hz, 2H), 7.55- 7.51 (m, 1H), 7.09 (d, J = 8.7 Hz, 2H), 6.92 (dd, J = 5.6, 2.6 Hz, 1H), 4.05 (s, 3H), 3.93 (s, 3H). ¹³C NMR (126 MHz, CDCl₃) δ 166.7, 159.8, 158.4, 155.5, 150.3, 149.0, 148.5, 131.0, 130.7, 130.2, 129.3, 127.1, 126.7, 125.9, 119.4, 114.0, 111.2, 106.8, 55.4, 55.4. HRMS (M + H⁺) for C₂₂H₁₉N₂O₂; Calculated: 343.1441, measured: 343.1442. |
| 4-(4-Methoxyphenyl)-2-(4-methoxypyridin-2- | |
| yl)quinoline (PPQN^{2,4-di-OMe}) | |
| 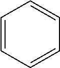 | Colourless oil (21.8 mg, 65%) ¹H NMR (500 MHz, CDCl₃) δ 7.34-7.29 (m, 4H), 7.26-7.18 (m, 6H), 4.02 (s, 2H). ¹³C NMR (126 MHz, CDCl₃) δ 141.1, 129.0, 128.5, 126.1, 42.0. |
| | GC-MS (EI, m/z) for C₁₃H₁₂; calculated: |
| | 168.1, measured: 168.1. Spectra |
| | consistent with the literature. |
| 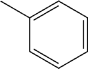 | |
| Diphenylmethane | |
| 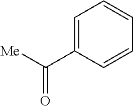 | Colourless oil (31.6 mg, 75%). ¹H NMR (500 MHz, CDCl₃) δ 7.91 (d, J = 8.4 Hz, 2H), 7.36-7.28 (m, 4H), 7.28- 7.22 (m, 1H), 7.22-7.17 (m, 2H), 4.06 (s, 2H), 2.60 (s, 3H). ¹³C NMR (126 MHz, CDCl₃) δ 197.8, 146.8, 140.1, 135.3, 129.1, 129.0, 128.7, 126.4, |
| | 41.9, 26.6 (One aromatic carbon was |
| | missing due to overlap). |
| | GC-MS (EI, m/z) for C₁₅H₁₄O, Calculated: |
| | 210.1, measured: 210.1. Spectra was |
| | consistent with the literature. |
| 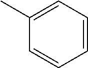 | |
| 1-(4-Benzylphenyl)ethan-1-one | |
| 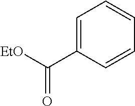 [SMILES: CCOC(=O)c1ccccc1] | Colourless oil (25.0 mg, 52%). ¹H NMR (500 MHz, CDCl₃) δ 7.99 (d, J = 8.4 Hz, 2H), 7.34-7.27 (m, 4H), 7.25 (d, J = 7.3 Hz, 1H), 7.22-7.17 (m, 2H), 4.39 (q, J = 7.1 Hz, 2H), 4.06 (s, 2H), 1.40 (t, J = 7.1 Hz, 3H). ¹³C NMR (126 MHz, CDCl₃) δ 166.6, 146.4, |
| | 140.2, 129.8, 129.0, 128.9, 128.6, 128.5, |
| | 126.4, 60.8, 41.9, 14.4. |
| | GC-MS (EI, m/z) for C₁₆H₁₆O₂, Calculated: |
| | 240.1, measured: 240.2. Spectra was |
| | consistent with the literature. |
| 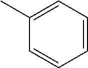 | |
| Ethyl 4-benzylbenzoate | |
| 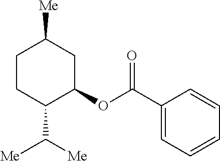 [SMILES: CC(C)[C@@H]1CC[C@@H](C)C[C@H]1OC(=O)c1ccccc1] | Colourless oil (42.7 mg, 61%). ¹H NMR (500 MHz, CDCl₃) δ 7.93 (d, J = 0.8 Hz, 1H), 7.92-7.87 (m, 1H), 7.39- 7.35 (m, 2H), 7.34-7.29 (m, 2H), 7.26- 7.19 (m, 3H), 4.94 (td, J = 10.9, 4.4 Hz, 1H), 4.06 (s, 2H), 2.18-2.10 (m, 1H), 2.00- 1.92 (m, 1H), 1.79-1.73 (m, 2H), 1.62- 1.56 (m, 2H), 1.19-1.10 (m, 2H), 0.95 (t, 7H), 0.81 (d, J = 6.9 Hz, 3H). ¹³C NMR (126 MHz, CDCl₃) δ 166.2, 141.4, 140.5, 133.3, 131.1, 130.1, 128.9, 128.6, |
| | 128.5, 127.4, 126.3, 74.8, 47.3, 41.7, 41.0, |
| | 34.3, 31.5, 26.5, 23.7, 22.1, 20.8, 16.6. |
| | HRMS (M + Na⁺) for C₂₄H₃₀O₂Na; |
| | Calculated: 373.2138, measured: |
| | 373.2139. |
| 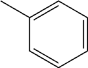 | |
| (1R,2S,5R)-2-isopropyl-5-methylcyclohexyl 3- | |
| benzylbenzoate | |
| 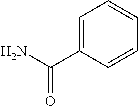 | White solid (19.8 mg, 47%). ¹H NMR (500 MHz, CDCl₃) δ 7.76 (d, J = 8.4 Hz, 2H), 7.35-7.29 (m, 4H), 7.27- 7.23 (m, 1H), 7.22-7.17 (m, 2H), 6.06 (br, 1H), 5.74 (br, 1H), 4.06 (s, 2H). ¹³C NMR (126 MHz, CDCl₃) δ 169.1, 145.6, 140.2, 131.2, 129.2, 128.9, 128.6, 127.6, 126.4, 41.8. |
| | GC-MS (EI, m/z) for C₁₄H₁₃NO; Calculated: |
| | 211.1, measured: 211.0. Spectra was |
| | consistent with the literature. |
| 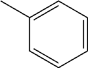 | |
| 4-Benzylbenzamide | |
| 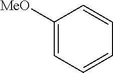 | Colourless oil (15.8 mg, 40%). ¹H NMR (500 MHz, CDCl₃) δ 7.35-7.29 (m, 2H), 7.26-7.20 (m, 4H), 6.84-6.80 (m, 1H), 6.80-6.74 (m, 2H), 3.99 (s, 2H), 3.80 (s, 3H). |
| | ¹³C NMR (126 MHz, CDCl₃) δ 159.7, 142.7, |
| | 140.9, 129.4, 128.9, 128.5, 126.1, 121.4, |
| | 114.8, 111.3, 55.2, 42.0. |
| | GC-MS (EI, m/z) for C₁₄H₁₄O Calculated: |
| | 198.1, measured: 198.0. Spectra was |
| | consistent with the literature. |
| 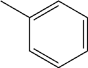 | |
| 1-Benzyl-3-methoxybenzene | |
| 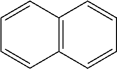 | Colourless oil (16.2 mg, 37%). ¹H NMR (500 MHz, CDCl₃) δ 7.87-7.77 (m, 3H), 7.69 (s, 1H), 7.52-7.45 (m, 2H), 7.40-7.31 (m, 3H), 7.30-7.24 (m, 3H), 4.20 (s, 2H). ¹³C NMR (126 MHz, CDCl₃) δ 141.0, 138.6, |
| | 133.7, 132.1, 129.1, 128.5, 128.1, 127.7, |
| | 127.7, 127.6, 127.1, 126.2, 126.0, 125.4, |
| | 42.2. |
| | GC-MS (EI, m/z) for C₁₇H₁₄; Calculated: |
| | 218.1, measured: 218.0. Spectra was |
| | consistent with the literature. |
| 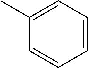 | |
| 2-Benzylnaphthalene | |
| 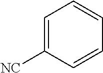 | Colourless oil (23.6 mg, 61%; or 32.8 mg, 85%). ¹H NMR (500 MHz, CDCl₃) δ 7.60 (d, J = 8.4 Hz, 2H), 7.35-7.25 (m, 5H), 7.18 (d, J = 6.9 Hz, 2H), 4.06 (s, 2H). ¹³C NMR (126 MHz, CDCl3) δ 146.7, |
| | 139.3, 132.3, 129.7, 129.0, 128.8, 126.7, |
| | 119.0, 110.1, 42.0. |
| | GC-MS (EI, m/z) for C₁₄H₁₁N; Calculated: |
| | 193.1, measured: 193.1. Spectra was |
| | consistent with the literature. |
| 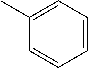 | |
| 4-Benzylbenzonitrile | |
| 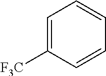 | Colourless oil (27.8 mg, 59%). ¹H NMR (500 MHz, CDCl₃) δ 7.56 (d, J = 8.2 Hz, 2H), 7.36-7.29 (m, 4H), 7.28- 7.22 (m, 1H), 7.20 (d, J = 6.7 Hz, 2H), 4.06 (s, 2H). ¹³C NMR (126 MHz, CDCl₃) δ 145.2, 140.0, |
| | 129.2, 128.9, 128.7, 128.5 (q, J = 33.2 Hz), |
| | 126.5, 125.4 (q, J = 3.7 Hz), 123.2, 41.7. |
| | ¹⁹F NMR (471 MHz, CDCl₃) δ −62.4. |
| | GC-MS (EI, m/z) for C₁₄H₁₁F₃; Calculated: |
| | 236.1, measured: 236.1. Spectra was |
| | consistent with the literature. |
| 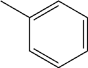 | |
| 1-Benzyl-4-(trifluoromethyl)benzene | |
| 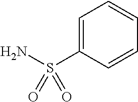 | White solid (26.2 mg, 53%). ¹H NMR (500 MHz, CDCl₃) δ 7.86 (d, J = 8.5 Hz, 2H), 7.40-7.30 (m, 4H), 7.28- 7.23 (m, 1H), 7.19 (d, J = 6.7 Hz, 2H), 4.78 (br, 2H), 4.07 (s, 2H). ¹³C NMR (126 MHz, CDCl₃) δ 146.7, 139.7, 139.6, 129.6, 129.0, 128.8, 126.7, 126.6, 41.8. |
| | GC-MS (EI, m/z) for C₁₃H₁₃NO₂S; |
| | Calculated: 247.1, measured: 247.0. |
| | Spectra was consistent with the literature. |
| 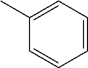 | |
| 4-Benzylbenzenesulfonamide | |
| 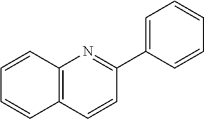 [SMILES: c1ccc(-c2ccc3ccccc3n2)cc1] | White solid (26 mg, 44%). ¹H NMR (500 MHz, CDCl₃) δ 8.23 (d, J = 7.8 Hz, 1H), 8.18 (d, J = 8.5 Hz, 1H), 8.12 (d, J = 8.2 Hz, 2H), 7.88 (d, J = 8.7 Hz, 1H), 7.84 (d, J = 8.1 Hz, 1H), 7.78-7.68 (m, 1H), 7.59-7.50 (m, 1H), 7.38 (d, J = 8.5 Hz, 2H), 7.36-7.30 (m, 2H), 7.28-7.20 (m, 3H), 4.10 (s, 2H). ¹³C NMR (126 MHz, CDCl₃) δ 157.3, 148.3, |
| | 142.5, 140.9, 137.7, 136.7, 129.7, 129.6, |
| | 129.5, 129.0, 128.5, 127.7, 127.5, 127.1, |
| | 126.2, 118.9, 41.8 (One aromatic carbon |
| | was missing due to overlap). |
| | HRMS (M + H⁺) for C₂₂H₁₈N; Calculated: |
| | 296.1434, found: 296.1433. |
| 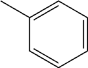 | |
| 2-(4-Benzylphenyl)quinoline | |
| 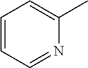 | Colourless oil (19.6 mg, 58%). ¹H NMR (500 MHz, CDCl₃) δ 8.58 (d, J = 3.7 Hz, 1H), 7.67-7.55 (m, 1H), 7.38- 7.19 (m, 5H), 7.18-7.06 (m, 2H), 4.19 (s, 2H). |
| | ¹³C NMR (126 MHz, CDCl₃) δ 161.0, 149.4, |
| | 139.5, 136.5, 129.1, 128.6, 126.4, 123.1, |
| | 121.2, 44.8. |
| | GC-MS (EI, m/z) for C₁₂H₁₁N; Calculated: |
| | 169.1, measured: 169.1. Spectra was |
| | consistent with the literature. |
| 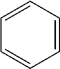 | |
| 2-Benzylpyridine | |
| 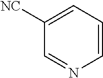 | Colourless oil (13.6 mg, 35%). ¹H NMR (500 MHz, CDCl₃) δ 8.74 (dd, J = 15.8, 2.2 Hz, 2H), 7.76-7.69 (m, 1H), 7.41- 7.35 (m, 2H), 7.34-7.29 (m, 1H), 7.22- 7.15 (m, 2H), 4.06 (s, 2H). ¹³C NMR (126 MHz, CDCl₃) δ 153.5, 150.2, |
| | 139.1, 138.0, 137.3, 129.1, 128.9, 127.2, |
| | 116.6, 109.9, 38.6. |
| | GC-MS (EI, m/z) for C₁₃H₁₀N₂; Calculated: |
| | 194.1, measured: 194.1. Spectra was |
| | consistent with the literature. |
| 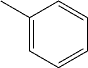 | |
| 5-Benzylnicotinonitrile | |
| 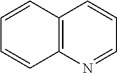 | Colourless oil (17.9 mg, 41%). ¹H NMR (500 MHz, CDCl₃) δ 8.85 (d, J = 2.3 Hz, 1H), 8.11 (d, J = 8.4 Hz, 1H), 7.94- 7.87 (m, 1H), 7.76 (d, J = 8.1 Hz, 1H), 7.71-7.65 (m, 1H), 7.57-7.51 (m, 1H), 7.37-7.32 (m, 2H), 7.29-7.25 (m, 3H), |
| | 4.20 (s, 2H). |
| | ¹³C NMR (126 MHz, CDCl₃) δ 152.2, 146.9, |
| | 139.7, 134.8, 133.9, 129.2, 129.0, 128.9, |
| | 128.8, 128.1, 127.5, 126.7, 126.6, 39.3. |
| | GC-MS (EI, m/z) for C₁₆H₁₃N; Calculated: |
| | 219.1, measured: 219.1. Spectra was |
| | consistent with the literature. |
| 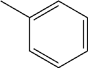 | |
| 3-Benzylquinoline | |
| 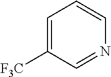 | Pale yellow oil (20.4 mg, 43%). ¹H NMR (500 MHz, CDCl₃) δ 8.84 (s, 1H), 7.83 (dd, J = 8.2, 2.6 Hz, 1H), 7.38-7.31 (m, 2H), 7.31-7.25 (m, 4H), 4.26 (s, 2H). ¹³C NMR (126 MHz, CDCl₃) δ 165.0, 146.3 (q, J = 4.1 Hz), 138.4, 133.6 (q, J = 3.4 Hz), |
| | 129.1, 128.8, 126.8, 124.4 (q, J = 33.0 Hz), |
| | 123.7 (q, J = 271.9 Hz), 122.8, 44.7. |
| | ¹⁹F NMR (471 MHz, CDCl₃) δ −62.3. |
| | GC-MS (EI, m/z) for C₁₃H₁₀F₃N; |
| | Calculated: 237.1, measured: 237.0. |
| | Spectra was consistent with the literature. |
| 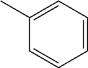 | |
| 2-Benzyl-5-(trifluoromethyl)pyridine | |
| 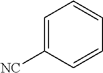 | Colourless oil (bromide: 37.6 mg, 91%; iodide: 37.3 mg, 90%). ¹H NMR (500 MHz, CDCl₃) δ 7.59 (d, J = 8.4 Hz, 2H), 7.30 (d, J = 8.2 Hz, 2H), 7.15 (d, J = 7.9 Hz, 2H), 7.07 (d, J = 8.1 Hz, 2H), |
| | 4.01 (s, 2H), 2.35 (s, 3H). |
| | ¹³C NMR (126 MHz, CDCl₃) δ 147.1, 136.3, |
| | 136.3, 132.3, 129.6, 129.5, 128.9, 119.1, |
| | 110.0, 41.6, 21.0. |
| | GC-MS (EI, m/z) for C₁₅H₁₃N; Calculated: |
| | 207.1, measured: 207.1. Spectra was |
| | consistent with the literature. |
| 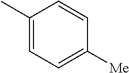 | |
| 4-(4-Methylbenzyl)benzonitrile | |
| 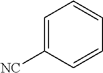 | Pale yellow solid (bromide: 50 mg, 93%; iodide: 49.6 mg, 92%). ¹H NMR (500 MHz, CDCl₃) δ 7.64-7.54 (m, 4H), 7.52-7.32 (m, 8H), 7.17 (d, J = 7.3 Hz, 1H), 4.13 (s, 2H). ¹³C NMR (126 MHz, CDCl₃) δ 146.6, 141.8, |
| | 140.9, 139.8, 132.4, 129.7, 129.2, 128.8, |
| | 127.9, 127.9, 127.5, 127.2, 125.6, 119.0, |
| | 110.2, 42.1. |
| | GC-MS (EI, m/z) for C₂₀H₁₅N; Calculated: |
| | 269.1, measured: 269.1. Spectra was |
| | consistent with the literature. |
| 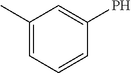 | |
| 4-([1,1′-Biphenyl]-3-ylmethyl)benzonitrile | |
| 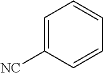 | Pale yellow solid (bromide: 36.7 mg, 65%; iodide: 33.2 mg, 59%). ¹H NMR (500 MHz, CDCl₃) δ 7.78 (d, J = 7.5 Hz, 1H), 7.75 (d, J = 7.8 Hz, 1H), 7.61 (d, J = 8.4 Hz, 2H), 7.55 (d, J = 7.5 Hz, 1H), |
| | 7.40-7.29 (m, 5H), 7.21 (d, J = 7.6 Hz, |
| | 1H), 4.13 (s, 2H), 3.89 (s, 2H). |
| | ¹³C NMR (126 MHz, CDCl₃) δ 147.0, 143.9, |
| | 143.2, 141.3, 140.4, 137.9, 132.4, 129.7, |
| | 127.7, 126.8, 126.7, 125.7, 125.0, 120.1, |
| | 119.8, 119.0, 110.1, 42.1, 36.8. |
| | HRMS (M + H⁺) for C₂₁H₁₆N; Calculated: |
| | 282.1277, measured: 282.1275. |
| 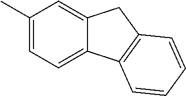 | |
| 4-((9H-fluoren-2-yl)methyl)benzonitrile | |
| 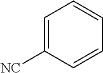 | Pale yellow solid (bromide: 43.8 mg, 90%; iodide: 45.8 mg, 94%). ¹H NMR (500 MHz, CDCl₃) δ 7.87-7.78 (m, 3H), 7.64 (s, 1H), 7.61 (d, J = 8.2 Hz, 2H), 7.54-7.46 (m, 2H), 7.35 (d, J = 8.2 |
| | Hz, 2H), 7.31-7.27 (m, 1H), 4.22 (s, 2H). |
| | ¹³C NMR (126 MHz, CDCl₃) δ 146.6, 136.8, |
| | 133.6, 132.4, 132.3, 129.8, 128.5, 127.7, |
| | 127.6, 127.4, 127.3, 126.3, 125.8, 119.0, |
| | 110.2, 42.1. |
| | GC-MS (EI, m/z) for C₁₈H₁₃N; Calculated: |
| | 243.1, measured: 243.1. |
| 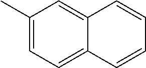 | |
| 4-(Naphthalen-2-ylmethyl)benzonitrile | |
| 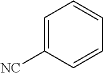 | Pale yellow solid (bromide: 39 mg, 86%; iodide: 37.5 mg, 82%). ¹H NMR (500 MHz, CDCl₃) δ 7.60 (d, J = 8.2 Hz, 2H), 7.36-7.22 (m, 4H), 7.11 (d, J = 8.4 Hz, 2H), 4.02 (s, 2H). |
| | ¹³C NMR (126 MHz, CDCl₃) δ 146.1, 137.8, |
| | 132.6, 132.4, 130.3, 129.6, 128.9, 118.9, |
| | 110.3, 41.3. |
| | GC-MS (EI, m/z) for C₁₄H₁₀ClN; |
| | Calculated: |
| | 227.1, measured: 227.1. Spectra was |
| | consistent with the literature. |
| 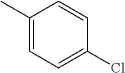 | |
| 4-(4-Chlorobenzyl)benzonitrile | |
| 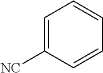 | Colourless oil (bromide: 42.4 mg, 95%; iodide: 43.4 mg, 97%). ¹H NMR (500 MHz, CDCl₃) δ 7.59 (d, J = 8.5 Hz, 2H), 7.29 (d, J = 8.5 Hz, 2H), 7.10 (d, J = 8.9 Hz, 2H), 6.87 (d, J = 8.7 Hz, 2H), 4.00 (s, 2H), 3.82 (s, 3H). |
| | ¹³C NMR (126 MHz, CDCl₃) δ 158.4, 147.3, |
| | 132.3, 131.4, 130.0, 129.5, 119.1, 114.2, |
| | 109.9, 55.3, 41.1. |
| | GC-MS (EI, m/z) for C₁₅H₁₃NO; Calculated: |
| | 223.1, measured: 223.0. Spectra was |
| | consistent with the literature. |
| 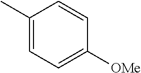 | |
| 4-(4-Methoxybenzyl)benzonitrile | |
| 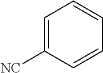 | White solid (46.4 mg, 98%). ¹H NMR (500 MHz, CDCl₃) δ 7.59 (d, J = 8.4 Hz, 2H), 7.29 (d, J = 8.2 Hz, 2H), 6.78 (d, J = 7.8 Hz, 1H), 6.67-6.62 (m, 2H), 5.95 (s, 2H), 3.96 (s, 2H). |
| | ¹³C NMR (126 MHz, CDCl₃) δ 148.0, 146.9, |
| | 146.4, 133.1, 132.3, 129.5, 121.9, 119.0, |
| | 110.1, 109.3, 108.4, 101.0, 41.6. |
| | GCMS (EI, m/z) for C₁₅H₁₁NO₂; Calculated: |
| | 237.1, measured: 237.0. Spectra was |
| | consistent with the literature. |
| 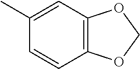 | |
| 4-(Benzo[d][1,3]dioxol-5-ylmethyl)benzonitrile | |
| 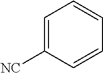 | Colourless oil (39.5 mg, 78%). ¹H NMR (500 MHz, CDCl₃) δ 7.59 (d, J = 8.4 Hz, 2H), 7.31 (d, J = 8.5 Hz, 2H), 6.38- 6.34 (m, 1H), 6.34-6.29 (m, 2H), 3.98 (s, 2H), 3.78 (s, 6H). ¹³C NMR (126 MHz, CDCl₃) δ 161.1, |
| | 146.4, 141.6, 132.3, 129.6, 119.0, 110.1, |
| | 107.2, 98.3, 55.3, 42.2. |
| | GC-MS (EI, m/z) for C₁₆H₁₅NO₂; |
| | Calculated: 253.1, measured: 253.1. |
| | Spectra was consistent with the literature. |
| 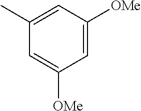 | |
| 4-(3,5-Dimethoxybenzyl)benzonitrile | |
| 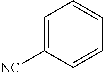 | Colourless oil (48.2 mg, 80%). ¹H NMR (500 MHz, CDCl₃) δ 7.60 (d, J = 8.5 Hz, 2H), 7.36 (d, J = 2.1 Hz, 1H), 7.28 (d, J = 8.5 Hz, 2H), 7.07 (dd, J = 8.4, 2.1 Hz, 1H), 6.86 (d, J = 8.4 Hz, 1H), 3.97 (s, 2H), 3.90 (s, 3H). |
| | ¹³C NMR (126 MHz, CDCl₃) δ 154.7, 146.4, |
| | 133.7, 132.9, 132.4, 129.5, 128.9, 118.9, |
| | 112.1, 111.9, 110.3, 56.3, 40.7. |
| | HRMS (M + Na⁺) for C₁₅H₁₂BrNONa; |
| | Calculated: 323.9995, measured: |
| | 323.9989 |
| 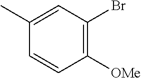 | |
| 4-(3-Bromo-4-methoxybenzyl)benzonitrile | |
| 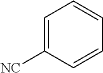 | White solid (NMR yield: 32%. pure spectra could not be obtained due to irremovable impurity). ¹H NMR (500 MHz, CDCl₃) δ 7.63 (d, J = 8.4 Hz, 4H), 7.29 (d, J = 8.2 Hz, 4H), 4.12 (s, 2H). |
| | ¹³C NMR (126 MHz, CDCl₃) δ 144.8, 132.6, |
| | 129.7, 118.7, 110.8, 41.9. |
| | GC-MS (EI, m/z) for C₁₅H₁₀N₂; Calculated: |
| | 218.1, measured: 218.1. Spectra was |
| | consistent with the literature. |
| 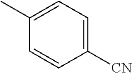 | |
| 4,4′-Methylenedibenzonitrile | |
| 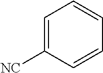 | Colourless oil (26.1 mg, 50%). ¹H NMR (500 MHz, CDCl₃) δ 7.62 (d, J = 8.4 Hz, 2H), 7.59 (d, J = 7.9 Hz, 2H), 7.30 (d, J = 7.9 Hz, 4H), 4.12 (s, 2H). ¹³C NMR (126 MHz, CDCl₃) δ 145.5, 143.4, |
| | 132.5, 129.7, 129.3, 129.0, 125.7 (q, J = |
| | 3.8 Hz), 125.7 (q, J = 272.0 Hz), 118.8, |
| | 110.6, 41.7. |
| | ¹⁹F NMR (471 MHz, CDCl3) δ −62.5. |
| | GC-MS (EI, m/z) for C₁₅H₁₀F₃N; |
| | Calculated: 261.1, measured: 261.0. |
| | Spectra was consistent with the literature. |
| 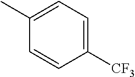 | |
| 4-(4-(Trifluoromethyl)benzyl)benzonitrile | |
| 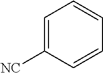 | Colourless oil (44.9 mg, 81%). ¹H NMR (500 MHz, CDCl₃) δ 7.61 (d, J = 8.5 Hz, 2H), 7.30 (d, J = 8.5 Hz, 2H), 7.23- 7.13 (m, 4H), 4.06 (s, 2H). ¹³C NMR (126 MHz, CDCl₃) δ 148.0, 148.0, 146.0, 138.1, 132.5, 130.2, 129.6, 121.3, |
| | 120.5 (q, J = 257.2 Hz), 118.8, 110.4, 41.2. |
| | ¹⁹F NMR (471 MHz, CDCl₃) δ −57.9. |
| | GC-MS (EI, m/z) for C₁₅H₁₀F₃NO; |
| | Calculated: 277.1, measured: 277.1. |
| | Spectra was consistent with the literature. |
| 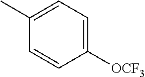 | |
| 4-(4-(Trifluoromethoxy)benzyl)benzonitrile | |
| 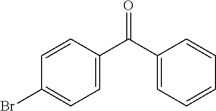 [SMILES: O=C(c1ccccc1)c1ccc(Br)cc1] | Colourless oil (27.4 mg, 31%). ¹H NMR (500 MHz, CDCl₃) δ 7.81 (d, J = 7.0 Hz, 2H), 7.77 (d, J = 8.4 Hz, 2H), 7.63- 7.56 (m, 1H), 7.52-7.46 (m, 2H), 7.34- 7.28 (m, 4H), 7.26-7.20 (m, 3H), 7.20- 7.13 (m, 3H), 4.05 (s, 2H), 3.99 (s, 2H). ¹³C NMR (126 MHz, CDCl₃) δ 196.4, 146.3, |
| | 141.1, 139.3, 137.9, 137.8, 135.5, 132.3, |
| | 130.5, 130.0, 129.2, 129.1, 128.9, 128.8, |
| | 128.5, 128.2, 126.1, 41.6 (One aliphatic |
| | carbon was missing due to overlap). |
| | HRMS (M + H⁺) for C₂₇H₂₂BrO; Calculated: |
| | 441.0849, measured: 441.0852. |
| 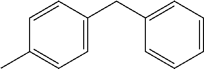 | |
| (4-(4-Benzylbenzyl)phenyl)(4- | |
| bromophenyl)methanone | |
| 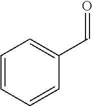 | White solid (16 mg, 41%). ¹H NMR (500 MHz, CDCl₃) δ 8.09-8.00 (m, 2H), 7.60-7.55 (m, 1H), 7.52-7.44 (m, 2H), 7.39-7.33 (m, 2H), 7.32-7.27 (m, 3H), 4.32 (s, 2H). ¹³C NMR (126 MHz, CDCl₃) δ 197.6, 136.6, 134.6, 133.2, 129.5, 128.7, 128.7, 128.6, 126.9, 45.5. |
| | GC-MS (EI, m/z) for C₁₄H₁₂O; Calculated: |
| | 196.1, measured: 196.1. Spectra was |
| | consistent with the literature. |
| 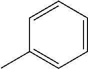 | |
| 1,2-Diphenylethan-1-one | |
| 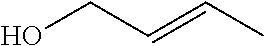 | Colourless oil (17.1 mg, 52%). ¹H NMR (500 MHz, CDCl₃) δ 7.33-7.29 (m, 2H), 7.24-7.17 (m, 3H), 5.85-5.65 |
| | (m, 2H), 4.11 (d, J = 4.4 Hz, 2H), 2.79- |
| | 2.66 (m, 2H), 2.45-2.34 (m, 2H), 1.28 (br, |
| | 1H). |
| | ¹³C NMR (126 MHz, CDCl₃) δ 141.7, 132.3, |
| | 129.6, 128.4, 128.3, 125.9, 63.7, 35.5, |
| | 34.0. |
| | HRMS (M + Na⁺) for C₁₁H₁₄Ona; Calculated: |
| | 185.0937, measured: 185.0940. Spectra |
| | was consistent with the literature. |
| 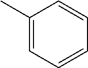 | |
| (E)-5-Phenylpent-2-en-1-ol | |
| 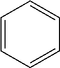 | Colorless oil (17.1 mg, 53%). ¹H NMR (500 MHz, CDCl₃) δ 7.30-7.24 (m, 2H), 6.97 (d, J = 7.8 Hz, 2H), 6.87- 6.82 (m, 1H), 3.21-3.13 (m, 4H), 1.77- 1.70 (m, 4H), 1.64-1.54 (m, 2H). ¹³C NMR (126 MHz, CDCl₃) δ 152.3, 129.0, |
| | 119.2, 116.5, 50.7, 25.9, 24.3. |
| | GC-MS (EI, m/z) for C₁₁H₁₅N; Calculated: |
| | 161.1, measured: 161.1. Spectra was |
| | consistent with the literature. |
| 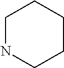 | |
| 1-Phenylpiperidine | |
| 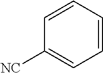 | Colourless oil (34.7 mg, 55%). ¹H NMR (500 MHz, CDCl₃) δ 7.75-7.67 (m, 2H), 7.32-7.24 (m, 2H), 4.59-4.44 (m, 1H), 3.66-3.43 (m, 2H), 2.48-2.34 (m, 1H), 2.23-2.12 (m, 1H), 2.11-1.96 |
| | (m, 2H), 1.48 (d, J = 15.1 Hz, 9H). |
| | ¹³C NMR (126 MHz, CDCl₃) δ 171.0, 170.8, |
| | 154.5, 154.1, 153.8, 153.6, 134.2, 133.8, |
| | 133.6, 122.7, 122.3, 118.3, 118.1, 116.3, |
| | 110.0, 109.8, 80.5, 80.3, 59.2, 59.1, 46.7, |
| | 46.5, 31.0, 30.0, 28.4, 24.6, 23.8 (Extra |
| | carbons due to rotamers). |
| | HRMS (M + Na⁺) for C₁₇H₂₀N₂O₄Na; |
| | Calculated: 339.1315, measured: |
| | 339.1314. Spectra was consistent with the |
| | literature. |
| 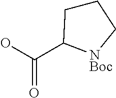 [SMILES: CC(C)(C)OC(=O)N1CCCC1C(=O)O] | |
| 1-(Tert-butyl)-2-(4-cyanophenyl)-pyrrolidine-1,2- | |
| dicarboxylate | |
| 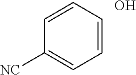 | White solid (21.4 mg, 90%). ¹H NMR (500 MHz, CDCl₃) δ 7.58 (d, J = 8.9 Hz, 2H), 6.95 (d, J = 7.2 Hz, 2H), 6.17 (br, 1H). ¹³C NMR (126 MHz, CDCl₃) δ 160.0, 134.3, 119.2, 116.4, 103.6. |
| | GC-MS (EI, m/z) for C₇H₅NO; Calculated: |
| 4-Hydroxybenzonitrile | 119.0, measured: 119.0. Spectra was |
| | consistent with the literature. |
| 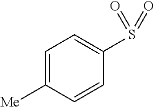 | White solid (H: 31.9 mg, 62%; O: 26.8 mg, 52%). ¹H NMR (500 MHz, CDCl₃) δ 8.05 (d, J = 8.7 Hz, 2H), 7.85 (d, J = 8.4 Hz, 2H), 7.80 (d, J = 8.7 Hz, 2H), 7.40-7.32 (m, 2H), 2.44 (s, 3H). ¹³C NMR (126 MHz, CDCl3) δ 146.3, 145.3, 137.2, 133.0, 130.3, 128.1, 128.1, |
| | 117.2, 116.7, 21.7. |
| | GC-MS (EI, m/z) for C₁₄H₁₁NO₂S; |
| | Calculated: 257.1, measured: 257.0. |
| | Spectra was consistent with the literature. |
| 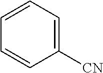 | |
| 4-Tosylbenzonitrile | |
| 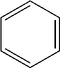 | White solid (28.4 mg, 51%). ¹H NMR (500 MHz, CDCl₃) δ 7.76-7.65 (m, 6H), 7.57 (m, 3H), 7.49 (m, 6H). ¹³C NMR (126 MHz, CDCl₃) δ 132.6 (d, J = 104.4 Hz), 132.1 (d, J = 9.8 Hz), 131.9 (d, |
| | J = 2.2 Hz), 128.5 (d, J = 12.4 Hz). |
| | ³¹P NMR (203 MHz, CDCl₃) δ 29.1. |
| | GC-MS (EI, m/z) for C₁₈H₁₅OP; Calculated: |
| | 278.1, found: 2781. Spectra was |
| | consistent with the literature. |
| 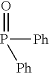 | |
| Triphenylphosphine oxide | |
| 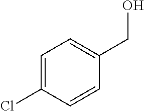 | Colourless oil (J: 22.6 mg, 62%; M: 19.4 mg, 53%). ¹H NMR (500 MHz, CDCl₃) δ 7.36-7.26 (m, 4H), 5.88-5.71 (m, 1H), 5.20 (d, J = 4.6 Hz, 1H), 5.17 (s, 1H), 4.78-4.71 (m, 1H), 2.58-2.44 (m, 2H), 2.11 (br, 1H). ¹³C NMR (126 MHz, CDCl₃) δ 142.3, 134.0, 133.2, 128.6, 127.2, 118.9, 72.6, 43.9. |
| | HRMS (M − H⁺) for C₁₀H₁₀ClO; Calculated: |
| | 181.0415, measured: 181.0420. Spectra |
| | was consistent with the literature. |
| 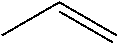 | |
| 1-(4-Chlorophenyl)but-3-en-1-ol | |
| 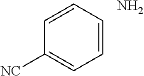 | White solid (13.2 mg, 56%). ¹H NMR (500 MHz, CDCl₃) δ 7.42 (d, J = 8.9 Hz, 2H), 6.66 (d, J = 8.7 Hz, 2H), 4.20 (br, 2H). ¹³C NMR (126 MHz, CDCl₃) δ 150.5, 133.8, 120.2, 114.5, 100.2. |
| | GC-MS (EI, m/z) for C₇H₆N₂; Calculated: |
| 4-Aminobenzonitrile | 181.1, measured: 181.1. Spectra was |
| | consistent with the literature. |
| 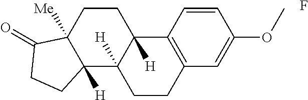 | White solid (27.2 mg, 45%). ¹H NMR (500 MHz, CDCl₃) δ 7.27 (d, J = 8.7 Hz, 1H), 6.94-6.87 (m, 1H), 6.85 (d, J = 2.9 Hz, 1H), 5.71 (d, J = 55.6 Hz, 2H), 2.97-2.89 (m, 2H), 2.60-2.49 (m, 1H), 2.48-2.39 (m, 1H), 2.34-2.25 (m, 1H), 2.25-2.12 (m, 1H), 2.10-1.91 (m, 3H), |
| | 1.67-1.47 (m, 6H), 0.94 (s, 3H). |
| (8R,9S,13S,14S)-3-(fluoromethoxy)-13-methyl- | ¹³C NMR (126 MHz, CDCl₃) δ 220.8, 154.8 |
| 6,7,8,9,11,12,13,14,15,16-decahydro- | (d, J = 3.5 Hz), 138.2, 135.0, 126.6, 116.8, |
| 17Hcyclopenta[a]phenanthren-17-one | 114.2, 100.9 (d, J = 218.2 Hz), 50.4, 48.0, |
| | 44.0, 38.2, 35.9, 31.6, 29.6, 26.4, 25.9, |
| | 21.6, 13.9. |
| | GC-MS (EI, m/z) for C₁₉H₂₃FO₂; |
| | Calculated: 302.2, measured: 302.1. |
| | Spectra was consistent with the literature. |
| 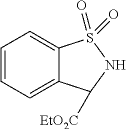 [SMILES: CCOC(=O)C1NS(=O)(=O)c2ccccc21] | White solid (40.4 mg, 61%). ¹H NMR (500 MHz, CDCl₃) δ 7.92 (d, J = 7.8 Hz, 1H), 7.81 (d, J = 7.6 Hz, 1H), 7.76- 7.70 (m, 1H), 7.68-7.62 (m, 1H), 7.36- 7.29 (m, 5H), 5.60 (s, 1H), 4.36-4.17 (m, 2H), 3.59 (d, J = 13.6 Hz, 1H), 3.22 (d, J = 13.6 Hz, 1H), 1.32 (t, J = 7.2 Hz, 3H). ¹³C NMR (126 MHz, CDCl₃) δ 169.1, 138.2, 135.6, 134.4, 133.5, 130.6, 130.5, 128.4, |
| | 127.7, 125.1, 121.6, 69.7, 63.5, 46.2, 14.1. |
| | GCMS (EI, m/z) for C₁₇H₁₇NO₄S; |
| | Calculated: 331.1, measured: 331.2. |
| | Spectra was consistent with the literature. |
| 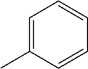 | |
| Ethyl 3-benzyl-2,3-dihydrobenzo[d]isothiazole-3- | |
| carboxylate 1,1-dioxide | |
| 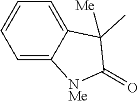 | White solid (33 mg, 59%). ¹H NMR (500 MHz, CDCl₃) δ 7.90-7.83 (m, 2H), 7.57-7.50 (m, 1H), 7.45-7.38 (m, 2H), 7.31-7.24 (m, 1H), 7.16 (d, J = 7.3 Hz, 1H), 7.02-6.97 (m, 1H), 6.92 (d, J = 7.8 Hz, 1H), 3.79-3.63 (m, 2H), 3.33 (s, 3H), 1.47 (s, 3H). |
| | ¹³C NMR (126 MHz, CDCl₃) δ 196.1, 180.6, |
| | 143.9, 136.4, 133.7, 133.2, 128.5, 128.0, |
| | 127.9, 122.2, 121.8, 108.2, 46.1, 45.3, |
| | 26.5, 25.0. |
| | GC-MS (EI, m/z) for C₁₈H₁₇NO₂; |
| | Calculated: 279.1, measured: 279.1. |
| | Spectra was consistent with the literature. |
| 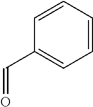 | |
| 1,3-Dimethyl-3-(2-oxo-2-phenylethyl)indolin-2- | |
| one | |

In conclusion, it is reported herein a well-tailored photoactive ligand, PPQN^{2,4-di-OMe}, that was designed for a wide range of metallaphotoredox cross-coupling reactions. The TM complexes of PPQN^{2,4-di-OMe}, including Fe, Co, Ni, and Cu, were highly enabling in photocatalytic C—C and C—X bond-forming transformations, either in a redox-neutral or net reductive fashion. These simple metal pyridyl catalysts were bifunctional, concurrently serving as PCs and traditional metal catalysts. Thus, such a synergistic activation mode increases the variety of base-metal photocatalysis and represents a complementary strategy for the current mainstay of binary meta llaphotoredox systems, which consist of two discrete catalytic entities for separate functions.

1. A process for alkylating a substrate with a photocatalytic system, the process comprising:
a) providing mixture comprising an acid, and the substrate, the substrate being an organic compound;
b) contacting an organophotoredox catalyst of formula Ia with the mixture of step a) 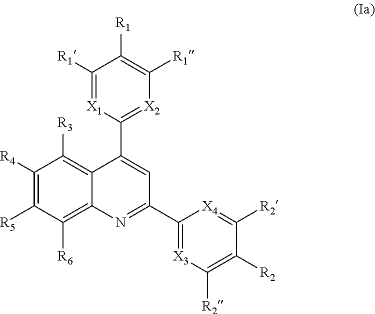
wherein R₁, R₁′, R₁″ are independently selected from hydrogen, substituted or unsubstituted alkyl, substituted or unsubstituted X-alkyl, chemical linker, or X-chemical linker with X being one of an oxygen, an amine or a sulfur, X₁, and X₂ are independently selected from CH or N, when X₁ is N, X₂ is CH, R₁ and R₁′ are hydrogen, when X₂ is N, X₁ is CH, R₁ and R₁″ are hydrogen, when X₁, and X₂ are both CH, R₁′ and R₁″ are hydrogen,
wherein R₂, R₂′, R₂″ are independently selected from hydrogen, substituted or unsubstituted alkyl, substituted or unsubstituted X-alkyl, chemical linker, or X-chemical linker with X being one of an oxygen,
an amine or a sulfur, X₃, and X₄ are independently selected from CH or N, when X₃ is N, X₄ is CH, R₂ and R₂″ are hydrogen, when X₄ is N, X₃ is CH, R₂ and R₂′ are hydrogen, when X₃, and X₄ are both CH, R₂′ and R₂″ are hydrogen,
wherein R₁, R₁′, R₁″, R₂, R₂′, R₂″ are not all hydrogen,
wherein R₃, R₄, R₅, and R₆ are independently selected from hydrogen, substituted or unsubstituted alkyl, substituted or unsubstituted alkenyl, substituted or unsubstituted cycloalkyl, substituted or unsubstituted cycloalkenyl, substituted or unsubstituted aryl, substituted or unsubstituted heteroaryl, substituted or unsubstituted heterocyclyl; and
c) activating the organophotoredox catalyst of step b) with a light irradiation to alkylate the substrate and form a carbon covalent bond.

2. The process according to claim 1, wherein the process is performed in an inert atmosphere.

3. The process according to claim 1, wherein the light irradiation has a wavelength of from 380 nm to 780 nm.

4. The process according to claim 1, wherein the organophotoredox catalyst is present in a concentration of at least 0.025 mol %.

5. The process of claim 1, further comprising after the step of contacting and before the step of activating, protonating the quinoline nitrogen of the organophotoredox catalyst.

6. The process according to claim 1, further comprising providing an alkylation precursor in the mixture.

7. The process according to claim [symbol] , wherein the alkylation precursor is an alkyltrifluoroborate salt, 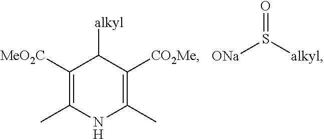
Boc₂(NH)—SO₂-alkyl, NH₃COO-alkyl,

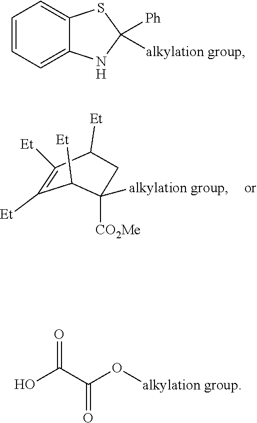

8. The process according to claim [symbol] , wherein the alkylation precursor comprises an alkyl moiety functionalized with one or more of an ester, a ketone, an ethereal, a carbamoyl, a benzyloxy, an allyloxy, and propargyloxy.

9. The process according to claim 1, wherein step b) further comprises contacting the organophotoredox catalyst with a co-catalyst comprising Ni, Co, Fe or Cu.

10. The process according to claim 1, wherein the acid is trifluoroacetic acid or HCl.

11. The process according to claim 1, wherein the organophotoredox catalyst is of formula Ib, Ic or Id 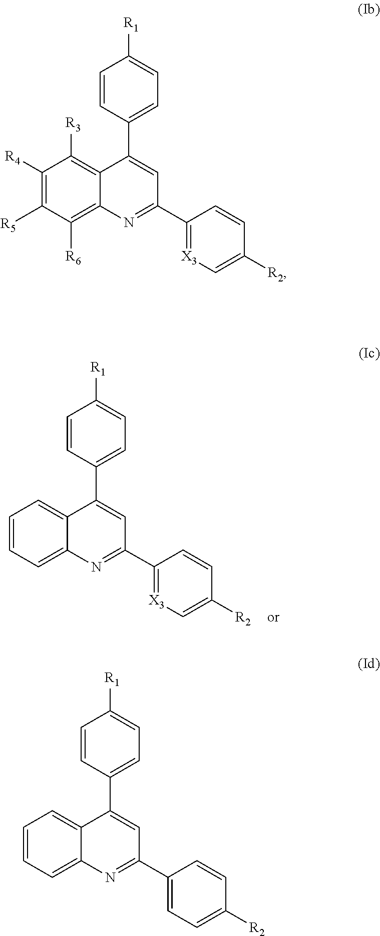
wherein X₃ is N or CH, R₁ and R₂ are not both H, and R₁, R₂ R₃, R₄, R₅, and R₆ are as defined in claim 1.

12. (canceled)

13. (canceled)

14. The process according to claim 1, wherein the organophotoredox catalyst is selected from the group consisting of:

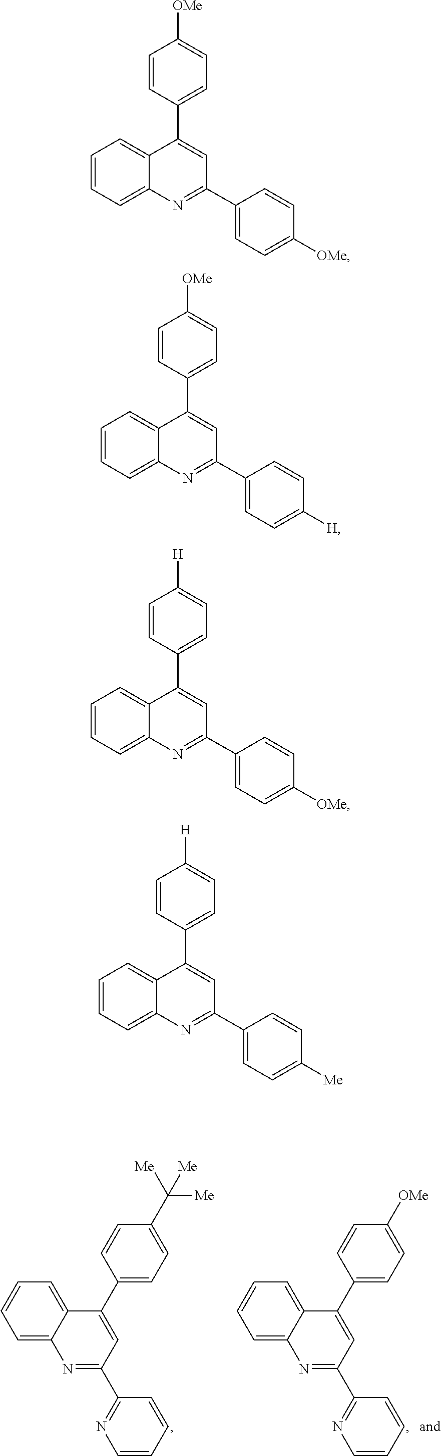

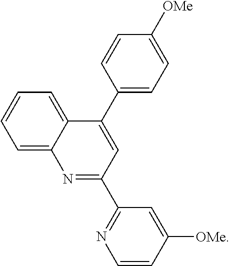 [SMILES: COc1ccc(-c2cc(-c3cc(OC)ccn3)nc3ccccc23)cc1]

15. The process according to claim 1, wherein the organophotoredox catalyst is

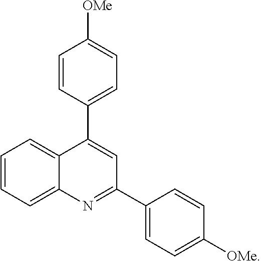 [SMILES: COc1ccc(-c2cc(-c3ccc(OC)cc3)c3ccccc3n2)cc1]

16. (canceled)

17. (canceled)

18. The process according to claim 1, wherein activating the organophotoredox catalyst further comprises obtaining an activated catalyst of formula IIa 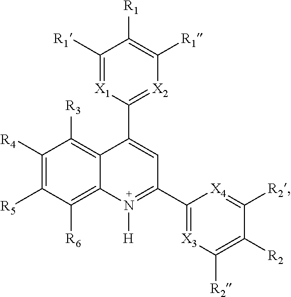
and wherein R₁, R₂, R₃ R₄, R₅, R₆, X₁, X₂, X₃, and X₄, are as defined in claim 1.

19. The process according to claim 1, wherein the mixture further comprises a co-catalyst selected from Ni, Cu, Co or Fe and X₃ is N.

20. The process according to claim 19, further comprising obtaining a metallophotoredox catalyst after the step of contacting the organophotoredox catalyst with the mixture.

21. The process of claim 20, wherein the metallophotoredox catalyst is of formula Ie: 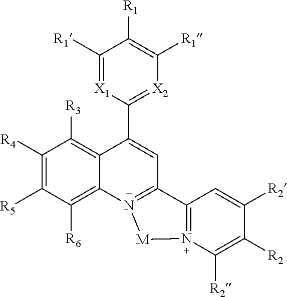
wherein R₁, R₁′, R₁″, R₂, R₂′, R₂″, R₃, R₄, R₅, R₆, X₁, and X₂ are as defined in claim 1.

22. The process of claim 20, wherein the metallophotoredox catalyst is of formula If: 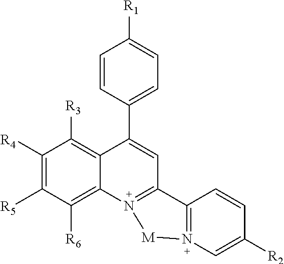
wherein R₁, R₂, R₃, R₄, R₅, and R₆, are as defined in claim 1.

23. The process of claim 20, wherein the metallophotoredox catalyst is of formula Ig: 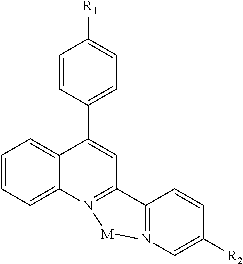
wherein R₁ and R₂ are as defined in claim 1.

24. A compound of formula Ia 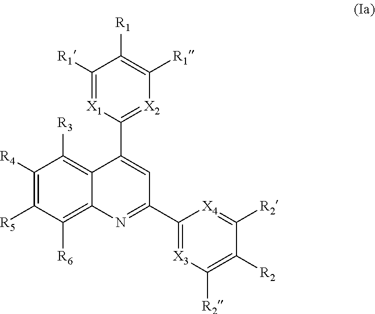
wherein R₁, R₁′, R₁″ are independently selected from hydrogen, substituted or unsubstituted alkyl, substituted or unsubstituted X-alkyl, chemical linker, or X-chemical linker with X being one of an oxygen, an amine or a sulfur, X₁, and X₂ are independently selected from CH or N, when X₁ is N, X₂ is CH, R₁ and R₁′ are hydrogen, when X₂ is N, X₁ is CH, R₁ and R₁″ are hydrogen, when X₁, and X₂ are both CH, R₁′ and R₁″ are hydrogen,
wherein R₂, R₂′, R₂″ are independently selected from hydrogen, substituted or unsubstituted alkyl, substituted or unsubstituted X-alkyl, chemical linker, or X-chemical linker with X being one of an oxygen, an amine or a sulfur, X₃, and X₄ are independently selected from CH or N, when X₃ is N, X₄ is CH, R₂ and R₂″ are hydrogen, when X₄ is N, X₃ is CH, R₂ and R₂′ are hydrogen, when X₃, and X₄ are both CH, R₂′ and R₂″ are hydrogen,
wherein R₁, R₁′, R₁″, R₂, R₂′, R₂″ are not all hydrogen,
wherein at least one of X₁, X₂ X₃, and X₄ is N,
and wherein R₃, R₄, R₅, and R₆ are independently selected from hydrogen, substituted or unsubstituted alkyl, substituted or unsubstituted alkenyl, substituted or unsubstituted cycloalkyl, substituted or unsubstituted cycloalkenyl, substituted or unsubstituted aryl, substituted or unsubstituted heteroaryl, substituted or unsubstituted heterocyclyl.

25. A compound selected from the group consisting of 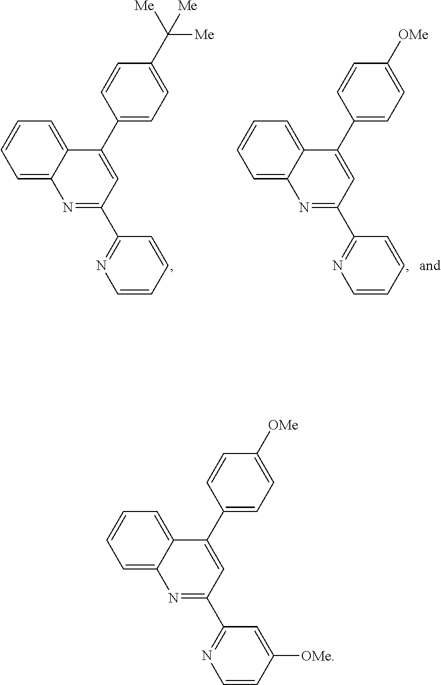 [SMILES: CC(C)(C)c1ccc(-c2cc(-c3ccccn3)nc3ccccc23)cc1 ; COc1ccc(-c2cc(-c3ccccn3)nc3ccccc23)cc1 ; COc1ccc(-c2cc(-c3cc(OC)ccn3)nc3ccccc23)cc1]
